SEQUENCE LISTING (78 sequences)

SEQ ID NO: 1 - protein, 597 aa, Escherichia coli
MSTQYETQGYTINNAGRRLVVDPITRIEGHMRCEVNINDQNVITNAVSCGTMFRGLEIILQGRDPRDAWAFVERICGVCTGVHALASVYAIEDAIGIKVPDNANIIRNIMLATLWCHDHLVHFYQLAGMDWIDVLDALKADPRKTSELAQSLSSWPKSSPGYFFDVQNRLKKFVEGGQLGIFRNGYWGHPQYKLPPEANLMGFAHYLEALDFQREIVKIHAVFGGKNPHPNWIVGGMPCAINIDESGAVGAVNMERLNLVQSIITRTADFINNVMIPDALAIGQFNKPWSEIGTGLSDKCVLSYGAFPDIANDFGEKSLLMPGGAVINGDFNNVLPVDLVDPQQVQEFVDHAWYRYPNDQVGRHPFDGITDPWYNPGDVKGSDTNIQQLNEQERYSWIKAPRWRGNAMEVGPLARTLIAYHKGDAATVESVDRMMSALNLPLSGIQSTLGRILCRAHEAQWAAGKLQYFFDKLMTNLKNGNLATASTEKWEPATWPTECRGVGFTEAPRGALGHWAAIRDGKIDLYQCVVPTTWNASPRDPKGQIGAYEAALMNTKMAIPEQPLEILRTLHSFDPCLACSTHVLGDDGSELISVQVR

SEQ ID NO: 2 - protein, 372 aa, Escherichia coli
MNNEETFYQAMRRQGVTRRSFLKYCSLAATSLGLGAGMAPKIAWALENKPRIPVVWIHGLECTCCTESFIRSAHPLAKDVILSLISLDYDDTLMAAAGTQAEEVFEDIITQYNGKYILAVEGNPPLGEQGMFCISSGRPFIEKLKRAAAGASAIIAWGTCASWGCVQAARPNPTQATPIDKVITDKPIIKVPGCPPIPDVMSAIITYMVTFDRLPDVDRMGRPLMFYGQRIHDKCYRRAHFDAGEFVQSWDDDAARKGYCLYKMGCKGPTTYNACSSTRWNDGVSFPIQSGHGCLGCAENGFWDRGSFYSRVVDIPQMGTHSTADTVGLTALGVVAAAVGVHAVASAVDQRRRHNQQPTETEHQPGNEDKQA

SEQ ID NO: 3 - protein, 567 aa, Escherichia coli
MSQRITIDPVTRIEGHLRIDCEIENGVVSKAWASGTMWRGMEEIVKNRDPRDAWMIVQRICGVCTTTHALSSVRAAESALNIDVPVNAQYIRNIILAAHTTHDHIVHFYQLSALDWVDITSALQADPTKASEMLKGVSTWHLNSPEEFTKVQNKIKDLVASGQLGIFANGYWGHPAMKLPPEVNLIAVAHYLQALECQRDANRVVALLGGKTPHIQNLAVGGVANPINLDGLGVLNLERLMYIKSFIDKLSDFVEQVYKVDTAVIAAFYPEWLTRGKGAVNYLSVPEFPTDSKNGSFLFPGGYIENADLSSYRPITSHSDEYLIKGIQESAKHSWYKDEAPQAPWEGTTIPAYDGWSDDGKYSWVKSPTFYGKTVEVGPLANMLVKLAAGRESTQNKLNEIVAIYQKLTGNTLEVAQLHSTLGRIIGRTVHCCELQDILQNQYSALITNIGKGDHTTFVKPNIPATGEFKGVGFLEAPRGMLSHWMVIKDGIISNYQAVVPSTWNSGPRNFNDDVGPYEQSLVGTPVADPNKPLEVVRTIHSFDPCMACAVHVVDADGNEVVSVKVL

SEQ ID NO: 4 - protein, 372 aa, Escherichia coli
MTGDNTLIHSHGINRRDFMKLCAALAATMGLSSKAAAEMAESVTNPQRPPVIWIGAQECTGCTESLLRATHPTVENLVLETISLEYHEVLSAAFGHQVEENKHNALEKYKGQYVLVVDGSIPLKDNGIYCMVAGEPIVDHIRKAAEGAAAIIAIGSCSAWGGVAAAGVNPTGAVSLQEVLPGKTVINIPGCPPNPHNFLATVAHIITYGKPPKLDDKNRPTFAYGRLIHEHCERRPHFDAGRFAKEFGDEGHREGWCLYHLGCKGPETYGNCSTLQFCDVGGVWPVAIGHPCYGCNEEGIGFHKGIHQLANVENQTPRSQKPDVNAKEGGNVSAGAIGLLGGVVGLVAGVSVMAVRELGRQQKKDNADSRGE

SEQ ID NO: 5 - protein, 618 aa, Ralstonia eutropha
MSAYATQGFNLDDRGRRIVVDPVTRIEGHMRCEVNVDANNVIRNAVSTGTMWRGLEVILKGRDPRDAWAFVERICGVCTGCHALASVRAVENALDIRIPKNAHLIREIMAKTLQVHDHAVHFYHLHALDWVDVMSALKADPKRTSELQQLVSPAHPLSSAGYFRDIQNRLKRFVESGQLGPFMNGYWGSKAYVLPPEANLMAVTHYLEALDLQKEWVKIHTIFGGKNPHPNYLVGGVPCAINLDGIGAASAPVNMERLSFVKARIDEIIEFNKNVYVPDVLAIGTLYKQAGWLYGGGLAATNVLDYGEYPNVAYNKSTDQLPGGAILNGNWDEVFPVDPRDSQQVQEFVSHSWYKYADESVGLHPWDGVTEPNYVLGANTKGTRTRIEQIDESAKYSWIKSPRWRGHAMEVGPLSRYILAYAHARSGNKYAERPKEQLEYSAQMINSAIPKALGLPETQYTLKQLLPSTIGRTLARALESQYCGEMMHSDWHDLVANIRAGDTATANVDKWDPATWPLQAKGVGTVAAPRGALGHWIRIKDGRIENYQCVVPTTWNGSPRDYKGQIGAFEASLMNTPMVNPEQPVEILRTLHSFDPCLACSTHVMSAEGQELTTVKVR

SEQ ID NO: 6 - protein, 360 aa, Ralstonia eutropha
MVETFYEVMRRQGISRRSFLKYCSLTATSLGLGPSFLPQIAHAMETKPRTPVLWLHGLECTCCSESFIRSAHPLAKDVVLSMISLDYDDTLMAAAGHQAEAILEEIMTKYKGNYILAVEGNPPLNQDGMSCIIGGRPFIEQLKYVAKDAKAIISWGSCASWGCVQAAKPNPTQATPVHKVITDKPIIKVPGCPPIAEVMTGVITYMLTFDRIPELDRQGRPKMFYSQRIHDKCYRRPHFDAGQFVEEWDDESARKGFCLYKMGCKGPTTYNACSTTRWNEGTSFPIQSGHGCIGCSEDGFWDKGSFYDRLTGISQFGVEANADKIGGTASVVVGAAVTAHAAASAIKRASKKNETSGSEH

SEQ ID NO: 7 - protein, 244 aa, Ralstonia eutropha
MSTKMQADRIADATGTDEGAVASGKSIKATYVYEAPVRLWHWVNALAIVVLAVTGFFIGSPPATRPGEASANFLMGYIRFAHFVAAYIFAIGMLGRIYWATAGNHHSRELFSVPVFTRAYWQEVISMLRWYAFLSARPSRYVGHNPLARFAMFFIFFLSSVFMILTGFAMYGEGAQMGSWQERMFGWVIPLLGQSQDVHTWHHLGMWFIVVFVIVHVYAAIREDIMGRQSVVSTMVSGYRTFKD

SEQ ID NO: 8 - protein, 347 aa, Ralstonia eutropha
MNAPVCTGLASAKPGVLNVLWIQSGGCGGCSMSLLCADTTDFTGMLKSAGIHMLWHPSLSLESGVEQLQILEDCLQGRVALHALCVEGAMLRGPHGTGRFHLLAGTGVPMIEWVSRLAAVADYTLAVGTCAAYGGITAGGGNPTDACGLQYEGDQPGGLLGLNYRSRAGLPVINVAGCPTHPGWVTDALALLSARLLTASDLDTLGRPRFYADQLVHHGCTRNEYYEFKASAEKPSDLGCMMENMGCKGTQAHADCNTRLWNGEGSCTRGGYACISCTEPGFEEPGHPFHQTPKVAGIPIGLPTDMPKAWFVALASLSKSATPKRVKLNATADHPLIAPAIRKTRLK

SEQ ID NO: 9 - protein, 485 aa, Ralstonia eutropha
MERLVVGPFNRVEGDLEVNLEVASGRVCSARVNATMYRGLEQILLHRHPLDALVYAPRVCGICSVSQSVAASRALADLAGVTVPANGMLAMNLMLATENLADHLTHFYLFFMPDFTREIYAGRPWHTDATARFSPTHGKHHRLAIAARQRWFTLMGTLGGKWPHTESVQPGGSSRAIDAAERVRLLGRVREFRCFLEQTLYAAPLEDVVALDSEVALWRWHAQAPQAGDLRCFLTIAQDAALDQMGPGPGTYLSYGAYPQPEGGFCFAQGVWRSAQGRLDALDLAAISEDATSAWLVDQGGARHPANGLTAPAPDKVGAYTWNKAPRLAGAVLETGAIARQLAGAQPLVRDAVARCGATVYTRVLARLVELARVVPLMEDWLQSLEIGAPYWASAHLPDQGAGVGLTEAARGSLGHWVSVRDGRIDNYQIVAPTSWNFSPRDIAGQPGAVEKALEGAPVLQGETTPVAVQHIVRSFDPCMVCTVH

SEQ ID NO: 10 - protein, 416 aa, Aquifex aeolicus
MKIEKLVLTRVEGEASLNLVWERGVIKDAKISFYSTRGIEKVLRERPFMDALVINPRICGICGHAHLIATVRAIENAIGIKEIPEKAKITRLVTQITEMVQNHVKWFYLFVMPDFLKFKESLSQFEPFKGERWKRAVQFSSQIVKIIALFGGQWPHSSYAVPGGITSNFSEREVLKALNLVKEGKTFFEKNVSEDLELFLNLCEEFNLLSIGKAYNRFLSGGGLAYCFNPSYKKGKGKVCKFNVRYVQELEAADYSKANPVRYKGLPYETGPLARELISKNPLVLKLYKNYGDSYAVRVAARLAEIKDLLEILEKLLKELMNHLEEPSCLWEDRSDESGEGFGVVEAARGTLIHRVVIEKGKIKDYKVITPSQWNLGPRCKKYLGVAEKAIVGLDSELKAQMVLRSFDLCSVCTTK

SEQ ID NO: 11 - protein, 353 aa, Aquifex aeolicus
METFWEVFKRHGVSRRDFLKFATTITGLMGLAPSMVPEVVRAMETKPRVPVLWIHGLECTCCSESFIRSATPLASDVVLSMISLEYDDTLSAAAGEAVEKHRERIIKEYWGNYILAVEGNPPLGEDGMYCIIGGRPFVEILKESAEGAKAVIAWGSCASWGCVQAAKPNPTTAVPIDKVIKDKPIIKVPGCPPIAEVMTGVIMYMVLFDRIPPLDSQGRPKMFYGNRIHDTCYRRSFFNAGQFVEQFDDEGAKKGWCLYKVGCRGPTTYNSCGNMRWYNGLSYPIQSGHGCIGCAENNFWDNGPFYERIGGIPVPGIESKADKVGAIAAAAAAGGAIIHGIASKIRKSGEKEE

SEQ ID NO: 12 - protein, 361 aa, Hydrogenovibrio marinus
MSSQVETFYEVMRRQGITRRSFLKYCSLTAAALGLSPAYANKIAHAMETKPRTPVIWLHGLECTCCSESFIRSAHPLAKDVVLSMISLDYDDTLMAASGHAAEAILDEIKEKYKGNYILAVEGNPPLNQDGMSCIIGGRPFSEQLKRMADDAKAIISWGSCASWGCVQAAKPNPTQATPVHKFLGGGYDKPIIKVPGCPPIAEVMTGVITYMLTFDRIPELDRQGRPKMFYSQRIHDKCYRRPHFDAGQFVEEWDDEGARKGYCLYKVGCKGPTTYNACSTVRWNGGTSFPIQSGHGCIGCSEDGFWDKGSFYSRDTEMNAFGIEATADDIGKTAIGVVGAAVVAHAAISAVKAAQKKGDK

SEQ ID NO: 13 - protein, 611 aa, Hydrogenovibrio marinus
MSVLNTPNHYKMDNSGRRVVIDPVTRIEGHMRCEVNVDENNVIQNAVSTGTMWRGLEVILRGRDPRDAWAFVERICGVCTGCHALASVRAVEDALDIKIPHNATLIREIMAKTLQIHDHIVHFYHLHALDWVNPVNALKADPQATSELQKLVSPHHPMSSPGYFKDIQIRIQKFVDSGQLGIFKNGYWSNPAYKLSPEADLMAVTHYLEALDFQKEIVKIHAIFGGKNPHPNYMVGGVPCAINIDGDMAAGAPINMERLNFVKSLIEQGRTFNTNVYVPDVIAIAAFYRDWLYGGGLSATNVMDYGAYPKTPYDKSTDQLPGGAIINGDWGKIHPVDPRDPEQVQEFVTHSWYKYPDETKGLHPWDGITEPNYELGSKTKGSRTNIIEIDESAKYSWIKSPRWRGHAVEVGPLARYILAYAQGVEYVKTQVHTSLNRFNAVCRLLDPNHKDITDLKAFLGSTIGRTLARALESEYCGDMMLDDFNQLISNIKNGDSSTANTDKWDPSSWPEHAKGVGTVAAPRGALAHWIVIEKGKIKNYQCVVPTTWNGSPRDPKGNIGAFEASLMGTPMERPDEPVEVLRTLHSFDPCLACSTHVMSEEGEEMATVKVR

SEQ ID NO: 14 - protein, 596 aa, Thiocapsa roseopersicina
MSVTTANGFELDTAGRRLVVDPVTRIEGHLRCEVNLDENNVIRNAVSTGTMWRGLEVILRGRDPRDAWAFTERICGVCTGTHALTSVRAVEDALGIPIPENANSIRNIMHVTLQAHDHLVHFYHLHALDWVDVVSALGADPKATSALAQSISDWPKSSPGYFRDVQNRLKRFVESGQLGPFMNGYWGSPAYKLPPEANLMAVTHYLEALDFQKEIVKIHTVYGGKNPHPNWLVGGMPCAINVDGTGAVGAINMERLNLVSSIIDQTIAFIDKVYIPDLIAIASFYKDWTYGGGLSSQAVMSYGDIPDHANDMSSKNLLLPRGAIINGNLNEIHEIDLRNPEEIQEFVDHSWFSYKDETRGLHPWDGVTEPNFVLGPNAVGSRTRIEALDEQAKYSWIKAPRWRGHAMEVGPLARYVIGYAKGIPEFKEPVDKVLTDLGQPLEAIFSTLGRTAARGLEASWAAHKMRYFQDKLVANIRAGDTATANVDNWDPKTWPKEARGVGTTEAPRGALGHWIVIKDGKIDNYQAVVPTTWNGSPRDPAGNIGAFEASLLNTPLAKADEPLEILRTLHSFDPCLACATHIMGPDGEELTRIKVR

SEQ ID NO: 15 - protein, 360 aa, Thiocapsa roseopersicina
MPTTETYYEVMRRQGITRRSFLKFCSLTATALGLSPTFAGKIAHAMETKPRIPVVWLHGLECTCCSESFIRSAHPLVSDVILSMISLDYTILIMAAAGHQAEAILEEVRHKHAGNYILAVEGNPPLNQDGMSCIIGGRPFLEQLLEMADSCKAVISWGSCASWGCVQAARPNPTRATPVHEVIRDKPVIKVPGCPPIAEVMTGVLTYILTFDRLPELDRQGRPLMFYGQRIHDKCYRRPHFDAGQFVESWDDEGARRGYCLYKVGCKGPTTYNACSTIRWNGGVSFPIQSGHGCIGCSEDGFWDKGSFYQHVTDTHAFGIEANADRTGIAVATRRGAAHRAHAAVSVVKRVQQKKEEDQS

SEQ ID NO: 16 - protein, 625 aa, Alteromonas macleodii
MENTASNNRLVVDPITRIEGHLRIEAEMDGNTIKQAFSSGTSVRGIELILQGRDPRDAWAFAQRICGVCTLVHGMASVRAVEDAIRKAWRSNAKLGVAIGKPSMTSMPKGPMQHGKKGHRQSRTSIGVLSEAEMAIPQNAQLIRNIMIATQYVHDHVMHFYHLHALDWVDVVSALDADPTRTAALAGQLSDYPRSSPGYFKDMKQKVKTLVESGQLGIFSNAYWGHPGYKLPPEVNLMALAHYLDALTWQREVVKVHTIFGGKNPHPNFVVGGVPSPINLNASTGINTSRLVQLQDAITQMKSFVDQVYYPDIVAIAGYYKEWGTRGEGLGNFLTYGDLPMTSMDDPDSFLFPRGAILGRDLSKVHDLDLDDPSEIQEFVSSSWYRYSGGNASGLHPFNGQTTLEYTGPKPPYKHLNVGAEYSWLKSPRWKGHAMEVGPLARVLMMYAKKDAAAQDIVNRSLSILDLETSALFSTLGRTLARAVETKIVVNQLQSWYDQLLDNIAKGDTDTFNPLYFDPTNWPIKGQGVGVMEAPRGALGHWLVMQNGKIENYQCVVPTTWNAGPRDPNSQAGAYEAALQDKHTLHDPDQPLEILRTLHSFDPCLACAVHVMDETGEERLRLKVR

SEQ ID NO: 17 - protein, 332 aa, Alteromonas macleodii
MALPTLNKQLQASGISRRTFLKFCATTASLLALPQSAVADLATALGNARRPSVIWLPFQECTGCTEAILRSHAPTLESLIFDHISLDYQHTIMAAAGEQAEDARRAAMNAHKGQYLLLVDGSVPVGNPGYSTISGMSNVDMLRESAKDAAGIIAIGTCASFGGIPKANPNPTGAVAVSDIITDKPIVNISGCPPLPIAITAVLVHYLTFKRFPDLDELQRPLAFFGESIHDRCYRRPFFEQRKFAKSFDDEGAKNGWCLFELGCKGPETFNACATVKWNQGTSFPIESGHPCLGCSEPDFWDKSSFYQALGPWEWYKSKPGKGAQKHAGKNS

SEQ ID NO: 18 - protein, 576 aa, Allochromatium vinosum
MSERIVVDPITRIEGHLRIEAQMDGATIAQAYSSGTMVRGIETILKGRDPRDAWAFVQRICGVCTLVHGIASVRAVEDALRIELPLNAQLIRNLMIGAQYIHDHVMHFYHLHALDWVDVVSALSADPRATSELAQSISAWPKSSPGYFADTQKRIKTFVESGQLGIFANGYWGHPAYRLPPEANLMAVAHYLEALAWQRDTAKFHAIFGGKNPHPNFVVGGVPSPIDLDSDSALNAKRLAEVRNLIQSMRTFVDQVYVPDTLAIAGFYKDWGERGEGLGNFLCYGDLPTGASLDPATFLFPRGAILDRDLSTIHEVDLEATGEIQEFVNHSWYEYSVGNDRGLHPYEGQTNLEYDRRGGVAPPYKQLDVSDGYSWLKAPRWKGRSVEVGPLARVLMLYATGHDQARELVDSTLSRLDLPVDALYSTLGRTAARALESKILVDAMQGWYDGLIANVKSGDTKTFNETLWEPSSWPSRAQGVGIMEAPRGALGHWIVIEDGRIANYQAVVPSTWNAGPRDGRGQAGAYEAALQDNHQLVDVKQPIEILRTIHSFDPCIACAVHLADPESGESFQVRVV

SEQ ID NO: 19 - protein, 314 aa, Allochromatium vinosum
PSVVWLSFQECTGCTESLTRAHAPTLEDLILDFISLDYHHTLQAASGEAAEAARLQAMDENRGQYLVIVDGSIPGPDANPGFSTVAGHSNYSILMETVEHAAAVIAVGTCAAFGGLPQARPNPTGAMSVMDLVRDKPVINVPGCPPIPMVITGVIAHYLVFGRLPEVDGYGRPLAFYGQSIHDRCYRRPFYDKGLFAESFDDEGAKQGWCLYRLGCKGPTTYNACATMKWNDGTSWPVEAGHPCLGCSEPQFWDAGGFYEPVSVPLTLGPATLLGAGAAGAVVGGGLAALSRKKGRDAAATRQPVTVDELEQKL

SEQ ID NO: 20 - protein, 372 aa, Salmonella enterica
MTGDNTLITSHGINRRDFMKLCAALAATMGLSSKAAAEMAESVSNPQRPPVIWIGAQECTGCTESLLRATHPTVENLVLETISLEYHEVLSAAFGHQVEENKHNALEKYKGQYVLVVDGSIPLKDNGIYCMVAGEPIVDHIRKAADGAAAIIAIGSCSAWGGVAAAGVNPTGAVSLQEVLPGKTVINIPGCPPNPHNFLATVAHIITYGTPPKLDAKNRPTFAYGRLIHEHCERRPHFDAGRFAKEFGDEGHRQGWCLYHLGCKGPETWGNCSTLQFCDVGGVWPVAIGHPCYGCNEEGIGFHKGIHQLAHVENQTPRSEKPDVNMKEGGNISAGAVGLLGGVVGLVAGVSVMAVRELGRQQKKDNADSRGE

SEQ ID NO: 21 - protein, 597 aa, Salmonella typhimurium
MSNQYQTQGYTVNDAGRRLIVDPITRIEGHMRCEVNIDEQNVITNAVSCGTMFRGLEIILQGRDPRDAWAFVERICGVCTGVHALASVYAIEDAIGIQVPDNANIIRNIMLATLWCHDHLVHFYQLAGMDWIDVLNALKADPRATSQLAQSLSAWPMSSPGYFFDVQNRLKKFVDGGQLGIFRNGYWGHPQYKLSPEANLMGFAHYLEALDFQREIVKIHTIFGGKNPHPNWIVGGMPCAINLDQSGAVGAINMERLNLVQSIITRTADFINNVMVPDALAIGQFNKAWSQIGTGLSDKCVLSYGAFPDIANDFSQQSLLMPGGAVINGDFKNVMPVDLADPQQIQEFVDHAWYRYPDDRLGRHPFDGITDPWYNPGDVKGSDTHIQQLNEQERYSWIKAPRWRGHAMEVGPLARTLIAYHKGDAATIESVDRMMSALKLPLSGIQSTLGRILCRAHEAQWAVGKLQYFFDRLMTNLKNGDLATANTEKWEPASWPQHCRGIGFTEAPRGALGHWASIRDQKIELYQCVVPTTWNASPRDPKKQIGAYEAALMGTQMAIPDQPLEILRTLHSFDPCLACSTHVLGDDGSELIAVQVR

SEQ ID NO: 22 - protein, 235 aa, Escherichia coli
MQQKSDNVVSHYVFEAPVRIWHWLTVLCMAVLMVTGYFIGKPLPSVSGEATYLFYMGYIRLIHFSAGMVFTVVLLMRIYWAFVGNRYSRELFIVPVWRKSWWQGVWYEIRWYLFLAKRPSADIGHNPIAQAAMFGYFLMSVFMIITGFALYSEHSQYAIFAPFRYVVEFFYWTGGNSMDIHSWHRLGMWLIGAFVIGHVYMALREDIMSDDTVISTMVNGYRSHKFGKISNKERS

SEQ ID NO: 23 - protein, 317 aa, Desulfovibrio vulgaris
MKISIGLGKEGVEERLAERGVSRRDFLKFCTAIAVTMGMGPAFAPEVARALMGPRRPSVVYLHNAECTGCSESVLRAFEPYIDTLILDTLSLDYHETIMAAAGDAAEAALEQAVNSPHGFIAVVEGGIPTAANGIYGKVANHTMLDICSRILPKAQAVIAYGTCATFGGVQAAKPNPTGAKGVNDALKHLGVKAINIAGCPPNPYNLVGTIVYYLKNKAAPELDSLNRPTMFFGQTVHEQCPRLPHFDAGEFAPSFESEEARKGWCLYELGCKGPVTMNNCPKIKFNQTNWPVDAGHPCIGCSEPDFWDAMTPFYQN

SEQ ID NO: 24 - protein, 567 aa, Desulfovibrio vulgaris
MSGCRAQNAPGGIPVTPKSSYSGPIVVDPVTRIEGHLRIEVEVENGKVKNAYSSSTLFRGLEIILKGRDPRDAQHFTQRTCGVCTYTHALASTRCVDNAVGVHIPKNATYIRNLVLGAQYLHDHIVHFYHLHALDFVDVTAALKADPAKAAKVASSISPRKTTAADLKAVQDKLKTFVESGQLGPFTNAYFLGGHPAYYLDPETNLIATAHYLEALRLQVKAARAMAVFGAKNPHTQFTVVGGVTCYDALTPQRIAEFEALWKETKAFVDEVYIPDLLVVAAAYKDWTQYGGTDNFITFGEFPKDEYDLNSRFFKPGVVFKRDFKNIKPFDKMQIEEHVRHSWYEGAEARHPWKGQTQPKYTDLHGDDRYSWMKAPRYMGEPMETGPLAQVLIAYSQGHPKVKAVTDAVLAKLGVGPEALFSTLGRTAARGIETAVIAEYVGVMLQEYKDNIAKGDNVICAPWEMPKQAEGVGFVNAPRGGLSHWIRIEDGKIGNFQLVVPSTWTLGPRCDKNKLSPVEASLIGTPVADAKRPVEILRTVHSFDPCIACGVHVIDGHTNEVHKFRIL

SEQ ID NO: 25 - protein, 644 aa, Clostridium beijerinckii
MGDNKKSFIQSALGSVFSVFSEEELKELSNGRKIAICGKVNNPGIIEVPEGATLNEIIQLCGGLINKSNFKAAQIGLPFGGFLTEDSLDKEFDFGIFYENIARTIIVLSQEDCIIQFEKFYIEYLLAKIKDGSYKNYEVVKEDITEMFNILNRISKGVSNMREIYLLRNLAVTVKSKMNQKHNIMEEIIDKFYEEIEEHIEEKKCYTSQCNHLVKLTITKKCIGCGACKRACPVDCINGELKKKHEIDYNRCTHCGACVSACPVDAISAGDNTMLFLRDLATPNKVVITQMAPAVRVAIGEAFGFEPGENVEKKIAAGLRKLGVDYVFDTSWGADLTIMEEAAELQERLERHLAGDESVKLPILTSCCPSWIKFIEQNYGDMLDVPSSAKSPMEMFAIVAKEIWAKEKGLSRDEVTSVAIMPCIAKKYEASRAEFSVDMNYDVDYVITTRELIKIFENSGINLKEIEDEEIDTVMGEYTGAGIIFGRTGGVIEAATRTALEKMTGERFDNIEFEGLRGWDGFRVCELEAGDIKLRIGVAHGLREAAKMLDKIRSGEEFFHAIEIMACVGGCIGGGGQPKTKGNKQAALQKRAEGLNNIDRSKTLRRSNENPEVLAIYEKYLDHPLSNKAHELLHTVYFPRVKKD

SEQ ID NO: 26 - protein, 567 aa, Clostridium beijerinckii
MSKYMVIDGNRIEFDKEKNILDLVRKAGIDLPTLCYYTDLSVYGACRMCVVEDERGSILTSCSTPPKDMMSIRTNTPKLQKYRKVILELLLATHCRDCTICEKNGKCKLQKLASRFGLTNIRFKSIEGKKDLDTSSKSIIRDPNKCILCGDCVRMCNEIQSVGAIDFANRGSNMVVSPAFGKSLAETDCVNCGQCATVCPTGAIVVKSDIKNVWKAIYNHKQRVIAQVAPAVRVALGEEFGIKPGENVMGKIVAAMRKLGFENIYDTSLSADLTVIEESKEFLKKLESDDNKFPLFTSCCPAWVRYVENKYTELLPYVSSCKSPMEMFGSVVKAYFKEKDSLENRETISVAVMPCTAKKAEAAREEFIRDNIPDVDYVITTAELCAMIKEIGIQFDEIEAEASDIPLSLYSGAGVIFGVTGGVTEAVIREVVKDKSSRVLKDIEFIGVRGMKGVKTCELQVKDESIRIGIVSGLRNAEDLIEKIKSGEEHFDFIEVMACPGGCIAGAGQPFGLMEEKNERAKGLYKIDKVTQIKRSEENLVVKSLYEGLLKNRTKELLHVHYDKSEH

SEQ ID NO: 27 - protein, 644 aa, Clostridium beijerinckii
MGDNKKSFIQSALGSVFSVFSEEELKELSNGRKIAICGKVNNPGIIEVPEGATLNEIIQLCGGLINKSNFKAAQIGLPFGGFLTEDSLDKEFDFGLFYENIARTIIVLSQEDCIIQFEKFYIEYLLAKIKDGSYKNYEVVKEDITEMFNILNRISKGVSNMREIYLLRNLAVTVKSKMNQKHNIMEEIIDKFYEEIEEHIEEKKCYTSQCNHLVKLTITKKCIGCGACKRACPVDCINGELKKKHEIDYNRCTHCGACVSACPVDAISAGDNTMLFLRDLATPNKVVITQMAPAVRVAIGEAFGFEPGENVEKKIAAGLRKLGVDYVFDTSWGADLTIMEEAAELQERLERHLAGDESVKLPILTSCCPSWIKFIEQNYGDMLDVPSSAKSPMEMFAIVAKEIWAKEKGLSRDEVTSVAIMPCIAKKYEASRAEFSVDMNYDVDYVITTRELIKIFENSGINLKEIEDEEIDTVMGEYTGAGIIFGRTGGVIEAATRTALEKMTGERFDNIEFEGLRGWDGFRVCELEAGDIKLRIGVAHGLREAAKMLDKIRSGEEFFHAIEIMACVGGCIGGGGQPKTKGNKQAALQKRAEGLNNIDRSKTLRRSNENPEVLAIYEKYLDHPLSNKAHELLHTVYFPRVKKD

SEQ ID NO: 28 - protein, 461 aa, Clostridium beijerinckii
MDIGFVNIDKELCTGCQQCVEVCPVNAIQGKKGQPQNIDYDVCVSCGQCIQVCNSYGFENRENSHLIEEKRRDRGVLESVKEPVFAAFNKGNAAKVKEALHDEELFTIVQCAPAVRVSLGEEFGLKAGSLTAGKMAAALRRLGFNRVYDTNFGADLTIMEEGSELIKRVTEGGELPMFTSCCPAWVKFMEQSYPELLNHLSSCKSPQQMAGTIFKTYGAKIDKVNPKKIYNVAIMPCTCKQFECDREEMQDSGFKDVDIVITTREFAQLIRDNEIDFKNLKDEEFDLPLGSYTGAGNIFGVTGGVMEAALRSGYEMLTKKSIPNLELNFVRGSEGIRVAEVKLPKITLKVAVVSGLKNVVQILEDIKEGKCDFDFIEVMTCPEGCVSGGGQPKFILDIDRRNALVSRKKGIYKHDSELEIRKSHENPFIKKLYEEFLIEPLGEKSHHLLHTKFVSRKKEEI

SEQ ID NO: 29 - protein, 450 aa, Clostridium pasteurianum
MKSEYNDLFKSLIDAYYKDDFDEFIKKALSDSTVNKEELSNIISSFCGVELKYTDKDTYIKDLKNAIKNYNSDHKIVTKIRDCSVDCADENGKTSCQKSCPFDAILIDEANKTSYIDKDLCTDCGFCVEGCPNGSILDKVEFIPLANLLKEKQPVIAAVAPAITGQFGDDVTIDQLRTAFKKVGFADMIEVAFFADMLTLKEACEFNAHVKSKDDLMITSCCCPMWVGMLKRVYKDMVKYVSPSVSPMIAAGRVIKTLNSNCKVVFVGPCIAKKAESKNKDIEGDIDFVLTFEEVKNIFESLNINPSELPEDPSTDYASREGRLYARTGGVSISVSEAVAKLFPEKKDLFKSVQANGVIECKKILEKAQNGEVAANFIEGMGCVGGCVGGPKALIPKEKGREKVNEFAENSNVKISLESDQMKKILNMLNITSAKDFMDEEKIKIFEREF

SEQ ID NO: 30 - protein, 497 aa, Chlamydomonas reinhardtii
MSALVLKPCAAVSIRGSSCRARQVAPRAPLAASTVRVALATLEAPARRLGNVACAAAAPAAEAPLSHVQQALAELAKPKDDPTRKHVCVQVAPAVRVAIAETLGLAPGATTPKQLAEGLRRLGFDEVFDTLFGADLTIMEEGSELLHRLTEHLEAHPHSDEPLPMFTSCCPGWIAMLEKSYPDLIPYVSSCKSPQMMLAAMVKSYLAEKKGIAPKDMVMVSIMPCTRKQSEADRDWFCVDADPTLRQLDHVITTVELGNIFKERGINLAELPEGEWDNPMGVGSGAGVLFGTTGGVMEAALRTAYELFTGTPLPRLSLSEVRGMDGIKETNITMVPAPGSKFEELLKHRAAARAEAAAHGTPGPLAWDGGAGFTSEDGRGGITLRVAVANGLGNAKKLITKMQAGEAKYDFVEIMACPAGCVGGGGQPRSTDKAITQKRQAALYNLDEKSTLRRSHENPSIRELYDTYLGEPLGHKAHELLHTHYVAGGVEEKDEKK

SEQ ID NO: 31 - protein, 717 aa, Chlorella variabilis
MEALVRRGLQSPDQALRLLGVARAICVGLSRSLPALAASAEQFAEQYPKLKLKVNGREVTVPEGTSVLNACREAGAYVPTLCTHPRLPTTPGTCRICMVETGGGQLKPACATPAWEGMEVQTATDKVQESIRGVLSLMKANHPSDCMNCDASGRCEFQDLISRYNVKDVLPKLKTYSHEWDAEVQADFEHFHDSSSTALTLDLEKCIKCGRCVTMCGQVQQMNVLGMINRSRMAHPGVLIEEALDHSKCIECGQCSSVCPVGAIVEHSEWRQVLDALENKQKVMVVQTAPSVRVSIGEELGLAPGTVETGQMVAAQRALGFDYVFDSDFSADLTIMEEGTELLQRLGAAWRAETAAQDAAAGSWAAAKQGHGEGEAHGHAPGPLPMFTSCCPAWINLVEKSYPELIPHLSSCKSPQMMMGAVVKHYWAKKKGLKPEDVCLVGIMPCTAKKHETERKEFRNENGAYDCDYVITTREFGHMLRHKKIPMPSLKPEEFDNPLGEATGAAALFGATGGVMEAAIRTAYEIAAGEPLPKLEVEAVRGVKGVKEATLTLPANDTTLKAGVAGKEIRVAVASGIGNARHLLQRIQAGEAHYDFVEVMACPGGCIGGGGQPKTHDPDAVLKRMGAIYQVDKSLALRKSHENPSIHKIYAEFLGQPGGELSHKLLHTHYTDHSVDTLPSVRELGGSGEVAKRAALTAAGEMRYKRIAMVGDPSAKR

SEQ ID NO: 32 - protein, 642 aa, Chlorella variabilis
MATIHINGHTVSVPEGTSILTAATQLGIHIPTLCTHPRLPTTPGTCRLCLVEVAGGALKPACATPVCHGLEVTTDSPQVKDSIRGVLALLKANHPADCMTCDVSGRCEFQARPGFWGGAWAGQRMACPRQRPMLPMLGCRCLKCGRCVTACGLVQEMDVLGMKGRSRERHPAVLTEAMDLSKCISCGQCAVMCPVGAITERAEWREVEDQLDAKRKAGRGAGRAGLMVCVTAPAVRVAIGEELGLAPGTITTGQMVAAQRQLGFDYVFDVNFGADLTIMEEGTELLQRLRHAWGLDLPAEGSGGAGAGPLPMFTSCCPGWVTACEKSFPELLPHLSTCKSPQQMMGAVVKSHFAAKLGKRAQDICLVSVMPCTAKKYEAERGEMVREGEGPDVDYVITTREFGRLLRERHIPLASLPESAFDNPLGEGSGAGVIFGNTGGVMEAALRTAYELAAGQPLPKLEEEAIRGLRGIKQATVTLPPTAPAGMASRQLRVAVASGIGQARHLLERMHTGHSPHFDFVEVMACPGGCVGGGGQPKSADPLVLLKRMGAVYSIDERSAIRKSHENPSIQKLYKACAEFLGEPGGSLSHQLLHTTYINRSTASQPTYTAFQRMDEPCNPKLQQAAAAGRGSSSSALPGTSA

SEQ ID NO: 33 - protein, 488 aa, Ralstonia eutropha
MSRKLVIDPVTRIEGHGKVVVHLDDDNKVVDAKLHVVEFRGFEKFVQGHPFWEAPMFLQRICGICFVSHHLCGAKALDDMVGVGLKSGIHVTPTAEKMRRLGHYAQMLQSHTTAYFYLIVPEMLFGMDAPPAQRNVLGLIEANPDLVKRVVMLRKWGQEVIKAVFGKKMHGINSVPGGVNNNLSIAERDRFLNGEEGLLSVDQVIDYAQDGLRLFYDFHQKHRAQVDSFADVPALSMCLVGDDDNVDYYHGRLRIIDDDKHIVREFDYHDYLDHFSEAVEEWSYMKFPYLKELGREQGSVRVGPLGRMNVTKSLPTPLAQEALERFHAYTKGRTNNMTLHTNWARAIEILHAAEVVKELLHDPDLQKDQLVLTPPPNAWTGEGVGVVEAPRGTLLHHYRADERGNITFANLVVATTQNNQVMNRTVRSVAEDYLGGHGEITEGMMNAIEVGIRAYDPCLSCATHALGQMPLVVSVFDAAGRLIDERAR

SEQ ID NO: 34 - protein, 209 aa, Ralstonia eutropha
MRAPHKDEIASHELPATPMDPALAANREGKIKVATIGLCGCWGCTLSFLDMDERLLPLLEKVTLLRSSLTDIKRIPERCAIGFVEGGVSSEENIETLEHFRENCDILISVGACAVWGGVPAMRNVFELKDCLAEAYVNSATAVPGAKAVVPFHPDIPRITTKVYPCHEVVKMDYFIPGCPPDGDAIFKVLDDLVNGRPFDLPSSINRYD

SEQ ID NO: 35 - protein, 488 aa, Rhodococcus opacus
MSTKLVIDPVTRIEGHGKVTVHLDDNNNVVDAHLHVVEFRGFEKLVQGHPFWEAPMLMQRICGICFVSHHLCGAKALDDMVGVGLKSGIDVTPTAEKIRRLGHYAQMLQSHATAYFYLIVPEMLFGMDAAPEQRNVLGLIEANPELVKRVVMLRKWGQEVIKAVFGRRMHGISSVPGGVNKNLSVAECQRFLKGEEGLPSVDEVIEYAQEGVQLFYDFHEQNRVQVDSFANVSALSMSLVDADGNVDYYHGKLRIIDDDKNVVQEFDYHDYLDHFSEAVEEWSYMKFPFLKALGRERGSVRVGPLGRLNVTNSLSTPLAQEALERFHAYTNGKANNMTLHTNWARAIEILHAAELIKELLNDPDLQKEQLLLTPADNAWTGEGVGVVEAPRGTLLHHYRADQEGDITFANLVVATTQNNQVMNRTVRSVAEDYLGGQGEVTEGMMNAIEVGIRAYDPCLSCATHALGQMPLIVSVHDTEGHVINERVR

SEQ ID NO: 36 - protein, 209 aa, Rhodococcus opacus
MKHSEKNEIASHELPTTPLDPVLAAGRESKIKVAMIGLCGCWGCTLSFLDMDERLLVLLDKVTLHRSSLSDIKRITERCAIGFIEGGVANEENIETLEHYRENCDVLISVGACAVWGGVPAMRNVFELKDCLSEVYIDSATSVPGAKPVVPFHPDIPRITDKVYPCHEVVKMDYFIPGCPPDADAIFKVLDDLVNGRPFDLPSSINQYD

SEQ ID NO: 37 - protein, 564 aa, Desulfovibrio fructosovorans
MAESKPTPQSTFTGPIVVDPITRIEGHLRIMVEVENGKVKDAWSSSQLFRGLEIILKGRDPRDAQHFTQRACGVCTYVHALASSRCVDDAVKVSIPANARMMRNLVMASQYLHDHLVHFYHLHALDWVDVTAALKADPNKAAKLAASIDTARTGNSEKALKAVQDKLKAFVESGQLGIFTNAYFLGGHKAYYLPPEVNLIATAHYLEALHMQVKAASAMAILGGKNPHTQFTVVGGCSNYQGLTKDPLANYLALSKEVCQFVNECYIPDLLAVAGFYKDWGGIGGTSNYLAFGEFATDDSSPEKHLATSQFPSGVITGRDLGKVDNVDLGAIYEDVKYSWYAPGGDGKHPYDGVTDPKYTKLDDKDHYSWMKAPRYKGKAMEVGPLARTFIAYAKGQPDFKKVVDMVLGKLSVPATALHSTLGRTAARGIETAIVCANMEKWIKEMADSGAKDNTLCAKWEMPEESKGVGLADAPRGSLSHWIRIKGKKIDNFQLVVPSTWNLGPRGPQGDKSPVEEALIGTPIADPKRPVEILRTVHAFDPCIACGVHVIEPETNEILKFKVC

SEQ ID NO: 38 - protein, 264 aa, Desulfovibrio fructosovorans
LTAKHRPSVVWLHNAECTGCTEAAIRTIKPYIDALILDTISLDYQETIMAAAGETSEAALHEALEGKDGYYLVVEGGLPTIDGGQWGMVAGHPMIETCKKAAAKAKGIICIGTCSPYGGVQKAKPNPSQAKGVSEALGVKTINIPGCPPNPINFVGAVVHVLTKGIPDLDENGRPKLFYGELVHDNCPRLPHFEASEFAPSFDSEEAKKGFCLYELGCKGPVTYNNCPKVLFNQVNWPVQAGHPCLGCSEPDFWDTMTPFYEQG

SEQ ID NO: 39 - protein, 582 aa, Clostridium acetobutylicum
MKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHE

SEQ ID NO: 40 - protein, 499 aa, Desulfomicrobium baculatum
MSQAATPAADGKVKISIDPLTRVEGHLKIEVEVKDGKVVDAKCSGGMFRGFEQILRGRDPRDSSQIVQRICGVCPTAHCTASVMAQDDAFGVKVTTNGRITRNLIFGANYLQSHILHFYHLAALDYVKGPDVSPFVPRYANADLLTDRIKDGAKADATNTYGLNQYLKALEIRRICHEMVAMFGGRMPHVQGMVVGGATEIPTADKVAEYAARFKEVQKFVIEEYLPLIYTLGSVYTDLFETGIGWKNVIAFGVFPEDDDYKTFLLKPGVYIDGKDEEFDSKLVKEYVGHSFFDHSAPGGLHYSVGETNPNPDKPGAYSFVKAPRYKDKPCEVGPLARMWVQNPELSPVGQKLLKELYGIEAKNFRDLGDKAFSIMGRHVARAEETWLTAVAVEKWLKQVQPGAETYVKSEIPDAAEGTGFTEAPRGALLHYLKIKDKKIENYQIVSATLWNANPRDDMGQRGPIEEALIGVPVPDIKNPVNVGRLVRSYDPALGCAVH

SEQ ID NO: 41 - protein, 283 aa, Desulfomicrobium baculatum
MTEGAKKAPVIWVQGQGCTGCSVSLLNAVHPRIKEILLDVISLEFHPTVMASEGEMALAHMYEIAEKFNGNFFLLVEGAIPTAKEGRYCIVGETLDAKGHHHEVTMMELIRDLAPKSLATVAVGTCSAYGGIPAAEGNVTGSKSVRDFFADEKIEKLLVNVPGCPPHPDWMVGTLVAAWSHVLNPTEHPLPELDDDGRPLLFFGDNIHENCPYLDKYDNSEFAETFTKPGCKAELGCKGPSTYADCAKRRWNNGINWCVENAVCIGCVEPDFPDGKSPFYVAE

SEQ ID NO: 42 - protein, 468 aa, Hydrogenophilus thermoluteolus
MTQHAPQAVSPRPSLPANATRRVAIDPLSRVEGHGKVTIWLDDDGQVVEARLHIVEFRGFEAFIVGRPYWEAPVVVQRLCGICPVSHHLAAAKALDRLVGVTQLPPTAEKMRRLMHYGQVLQSHALHFFYLAAPDLLLGFSADPAQRNVFGLAAQKRELARQGILVRQFGQECIEATAGKRIHGTSAVPGGIHKNLSRRERMALLSRAPEIRSWCEAAVALIERLFTEHAPFFAQFGSFQTKTFSLVAADGSLDLYDGTFRVKEANGAILIDHYDPNDYDQLLVEAVRPWSYMKFPYLKAYGEPDGFYRVGPSARLINCDRLTTARAEAARQRFLTFDQGTVAHSTLGYHWARLIEMLHCAELIEALLTDADLEGGELRARGQRQHRGVGVIEAPRGTLIHHYEVGDDDLITYCNLIVSTTHNNAVMNQAVTTAAKAFLSGVTLTEALLNHIEVAVRAFDPCLSCATH

SEQ ID NO: 43 - protein, 189 aa, Hydrogenophilus thermoluteolus
MTSAAPSAMPPRKIRIATASLAGCFGCHMSFADIDTRLLALAEWVTFDRSPLTDWKTVGECDIALIEGGVCNAENVEVLRAYRRAARILVAVGACAINGGLPAQRNQHRVERLLTQVFEADRHLAPGSRVPNDPELPLLLEHVHPIHEIVRVDYYLPGCPPTAEVIWTFLTDLLVGREPHFPYPTLRYD

SEQ ID NO: 44 - protein, 264 aa, Desulfovibrio gigas
LTAKKRPSVVYLHNAECTGCSESVLRTVDPYVDELILDVISMDYHETLMAGAGHAVEEALHEAIKGDFVCVIEGGIPMGDGGYWGKVGGRNMYDICAEVAPKAKAVIAIGTCATYGGVQAAKPNPTGTVGVNEALGKLGVKAINIAGCPPNPMNFVGTVVHLLTKGMPELDKQGRPVMFFGETVHDNCPRLKHFEAGEFATSFGSPEAKKGYCLYELGCKGPDTYNNCPKQLFNQVNWPVQAGHPCIACSEPNFWDLYSPFYSA

SEQ ID NO: 45 - protein, 536 aa, Desulfovibrio gigas
MSEMQGNKIVVDPITRIEGHLRIEVEVEGGKIKNAWSMSTLFRGLEMILKGRDPRDAQHFTQRACGVCTYVHALASVRAVDNCVGVKIPENATLMRNLTMGAQYMHDHLVHFYHLHALDWVNVANALNADPAKAARLANDLSPRKTTTESLKAVQAKVKALVESGQLGIFTNAYFLGGHPAYVLPAEVDLIATAHYLEALRVQVKAARAMAIFGAKNPHTQFTVVGGCTNYDSLRPERIAEFRKLYKEVREFIEQVYITDLLAVAGFYKNWAGIGKTSNFLTCGEFPTDEYDLNSRYTPQGVIWGNDLSKVDDFNPDLIEEHVKYSWYEGADAHHPYKGVTKPKWTEFHGEDRYSWMKAPRYKGEAFEVGPLASVLVAYAKKHEPTVKAVDLVLKTLGVGPEALFSTLGRTAARGIQCLTAAQEVEVWLDKLEANVKAGKDDLYTDWQYPTESQGVGFVNAPRGMLSHWIVQRGGKIENFQLVVPSTWNLGPRCAEGKLSAVEQALIGTPIADPKRPVEILRTVHSYDPCIACGVH

SEQ ID NO: 46 - protein, 412 aa, Pyrococcus furiosus
MIIELDEFTRVEGNGKAEIVIENGEVKDARVKIVEGPRFFEILTLGRDYWDVPDLEARICAICYIAHSVASVRAIEKALGIDVPESVEKLRELALWGEIIESHALHLYLLALPDVFGYPDAISMIPRHGELVKEGLTIKAFGNAIRELIGGREIHGINIKPGGFGRYPSEEELEKIAEHSKSLIKFARRIVGIFASQEAGGAVGEVLMATSDYLWGDELIINGERVQYYEVDEVPVGYSFAKHSYYKGNPVFVGALPRLLLKGESIEGEAARMLEEYRDKLESKYVIYNNLAQAIELLYALERVPQLVEEILSEGIERGNGEISQESGEGVGYVEAPRGVLVHHYRIENGKVVWSNTITPTAFNQRLMELSLLEEAKRLYGSESEENMKKRLEVIVRAFDPCISCSVHFVKL

SEQ ID NO: 47 - protein, 629 aa, Desulfovibrio vulgaris
MSSSKTIRSRSIWDDAHAMLEKAKAEGISTVWDRAAEQTPACKFCELGTTCRNCIMGPCRIANRKDGKMRLGVCGADADVIVARNFGRFIAGGAAGHSDHGRDLIETLEAVAEGKAPGYTIRDVAKLRRIAAELGVADAATRPAHDVAADLVTICYNDFGSRRNALAFLARAPQVRRDLWQRLGMTPRGVDREIAEMMHRTHMGCDNDHTSLLVHAARTALADGWGGSMIGTELSDILFGTPRPRQSTVNLGVLRKDAVNILVHGHNPVVSEMILAATREPAVRQAAQDAGAADINVAGLCCTGNELLMRQGIPMAGNHLMTELAIVTGAADAIVADYQCIMPSLVQIAACYHTRFVTTSPKGRFTGATHVEVHPHNAQERCREIVMLAIDAYTRRDPARVDIPSQPVSIMSGFSNEAILEALGGTPKPLIDAVVAGQIRGFVGIVGCNNPKIRQDSANVTLTRELIRRDIMVLATGCVTTAAGKAGLLVPEAASKAGEGLAAVCRSLGVPPVLHMGSCVDNSRILQLCALLATTLGVDISDLPVGASSPEWYSEKAAAIAMYAVASGIPTHLGLPPNILGSENVTAMALHGLQDVVGAAFMVEPDPVKAADMLEAHIVARRARLGLTS

SEQ ID NO: 48 - protein, 629 aa, Desulfovibrio vulgaris
MNSGTPDTERTLWEDARQMLRKAEAENIPTAWDRMREQTPHCKFCELGTTCRNCVMGPCRIANRKDGKMRYGVCGADADVIVARNFGRFIAGGAAGHSDHGRDLIETLEAVVEGHAPGYTIRDEAKLRRIAGELGVADADTRPTLDVARDLVDVCYADFGSRRTELAFLKRAPKARRELWARLGMTPRGIDREIAEMMHRTHMGCDNDPASLLTHAARTALADGWGGSMIGTELSDILFGTPMPRQSTVNLGVLREDAVNILVHGHNPVVSEMILAAAREPAMQQAARDAGAAALNVAGLCCTGNELLMRQGIPMAGNHLMTELAIVTGAADAIVADYQCIMPSLVRIAACYHTRFVTTSSKGRFTGATHIEVHPHNAQEKCREIVQLAIEAFTRRDPARVSIPVQPVPITTGFSNEAILAALGGTPAPLLDAVKAGQIRGFVGIVGCNNPKIMQDSGNVGLAKELIRRDIMVLATGCVTTAAGKAGLLMPEAADMAGPGLSAVCKALGVPPVLHMGSCVDNSRILQLCGLLADELGVDISDLPVGASSPEWYSEKAAAIGLYAVASGIPTHLGLPPNILGSDVVTGMAVDGLNGLVGAAFMVEADPVKAADLLEAHIVARRQKLGLSA

SEQ ID NO: 49 - protein, 631 aa, Desulfovibrio psychrotolerans
MTREQRSVEELSIWEDARSMLRKARAEGVETAWDRLEQQTPHCTFCELGTTCRNCVMGPCRISPKATKSGKLQRGVCGADADVIVARNFGRFIAGGSAGHSDHGRDLIEVLEAIVEGETTDYAIRDEAKLRRIAAELGIDAAERPLMDVARDLVDECYSDFGSRRKSVGFLARAPEKRRALWEKLGMTPRGVDREIAEMMHRTHMGCDNDAPNTLIHAARCALADGWSGSMIGTELSDIIFGVPTPSMSTANLGILKKDTVNILVHGHNPVVSEMILAAAREPQLIAAAKAHGASGITVGGLCCTGNELLMRQGIPMAGNHLMTELAIVTGAVEAVVVDYQCIMPSLVQIAGCYHTRFITTAGKARFTGAIHFDIHPHNAMEEARKIVQMAVDAFAERDPKRVEIPVEPVRIMTGFSNEAILNALGGSLTPLLDAVKAGSIRGFVAIVGCNNPKIQQDSANVGLAKALIERDIMVLATGCVTTAAGKAGLLVPEAASMAGPGLKAVCTALGIPPVLHMGSCVDNSRIMHLCGLIANELGVDISDLPVGASSPEWYSEKAAAIGMYAVASGVYTHLGLPPNILGSQTVTDLALNGLEGLVGASFAVEPDPLKAADLLDARIRAKREALGLKP

SEQ ID NO: 50 - protein, 629 aa, Desulfoluna spongiiphila
MAREKRKIEELSMWEDARAMIRKAQAEGIETVWDRLEEQTPHCRFCELGTTCRNCTMGPCRISPKKGGKMQRGVCGADADVVVARNFGRFIAGGAAGHSDHGRDLIEVLAGVAEGECPDYGIRDKAKLVRIAEELGIKSEGRSEMDIAGDLAEVLFGDFGSRKAEVAFLKRAPESRRETWKQLGMTPRGVDREIAEMMHRTHMGCDNDAPSTLIHAARTALADGWAGSMIGTELSDIIFGTPTPSESTANLGVLKKDQVNILVHGHNPVVSEMILAAARMPELVARAKDVGASGINVGGLCCTGNELLMRQGIPMAGNHLMTELSIVTGAVEAVVVDYQCIMPSMVQISGCYHTRFITTSGKARFTGATHFDIHPENAMEKAREIVALAVDAFAERDASRVEIPHEPVPIMTGFSNEAILAAVGGTPDPLIDAMKRGAIRGVVGIVGCNNPKFTQDSMNVGLAKALLKKDILVLVTGCVTTAAGKAGLLMPEGAEMAGPGLKEVCGALGIPPVLHMGSCVDNARILQLCALLAETCGVDISDLPVAASSPEWYSEKAAAIGLYAVASGIETHLGHPPNILGSDTVTHLATEGLEDLVGARFIIEKDPEEAAERLDKRITAKRVGLGWNP

SEQ ID NO: 51 - protein, 629 aa, Halodesulfovibrio spirochaetisodalis
MAKEPKKPEELSIWQDAHAMIRKAKAESIETAWDRLERQTPHCKFCDLGTTCRNCIMGPCRISTKPGAKMNLGVCGADADVVVARNFGRFIAGGSAGHSDHGRDLIEVLEAIVDGDTKHYTITEPEKLIRLAAEVGIATEGRELNDVANDLVDECYKDFGSRRSSLAFLSRAPEQRRALWDRVGITPRGVDREIAEMMHRTHMGCDNDAPNTLLHSARCALSDGWGGSMIGTELSDVIFGTPTPSRSTSNLGVIKEDKVNILVHGHNPVVSEMILAAARRPELVEKAKAAGAAGINVAGLCCTGNELLMRQGIPMAGNHLMTELAIVTGAVEAVVVDYQCIMPSLVTAAHCYHTRFITTAEKAKFTGAMHFEVHPHNALEQATAIVEEAIRAYTERDKGRVEIPAESVEIMTGFSNEAILSALGGTLTPLLDAIKAGKIRGIVGIVGCNNPKIKQDSANVGLAKELLKRDILVLVTGCVTTAAGKAGLLVPEGIEMAGEGIKEVCGALGIPPVLHLGSCVDNSRIMHLCGLVAKELGVDISDLPVGASSPEWYSEKAAAIGMYAVASGIMTHLGLPPNILGSETVTNIALEGLEDIVGAHFVVEDDYVKAAELLDARIRMKRVGLGLSE

SEQ ID NO: 52 - protein, 631 aa, Desulfovibrio desulfuricans
MDDRQTDINNMSIWEDAQKMLHKARAEGIETAWDRLAQQTPHCRFCELGTTCRNCVMGPCRISAKAAPGGKLSRGVCGADAHVIVARNFGRFIAGGSAGHSDHGRDVIETLEAVVEGKAEGYEIRDPAKLLRIAGELGIAVDGLAVNEVAALVVDACYSDFGSRRGAVNFISRVPAKRRQVWEKLGITPRGVDREIAEMMHRTHMGCDNDAPNTMLHAARCALSDGWAGSMIATELCDILFGTPSPRMSTVNLGVIKKETVNILVHGHNPVVSEMILAVSREPEVLEQARQAGAAGITVAGLCCTGNELLMRQGIPMAGNHLMTELAIVTGAVEAVVVDYQCIMPSLVQVAACYHTRFIDTAPKARFTGAVHMNFTPENARQEASAILHMAIEAFAARDPGRVDIPVSPVNIMTGFSNEAILEALGGSLDPLLDAVKAGTVRGFAAVVGCNNPKVRQDSANVGIIKELIKKDIMVLVTGCITTAAGKAGLLLPEGAAMAGPGLQKLCASLGIPPVLHMGSCVDNARIMHLCGLIANSLGVDISDLPVVASAPEWYSEKAAAIGLYAVASGVYTHLGLPPNILGSDVVTDIALNGLEGLVGASFVVEADPVKAADLLDRRIRTKRSALGLDA

SEQ ID NO: 53 - protein, 627 aa, Desulfurivibrio alkaliphilus
MAEKNKKSIEEKSLWEDARAMLRKAEQDGVETAWDRLEQQTPHCKFCELGTSCRNCVMGPCRITAKMPRGVCGADADVVVARNFGRFIAGGSAGHSDHGRDLIEVLEAIVEGETKDYQIKDEAKLRRIAGELGVADAGGGALAAVARAVMEILYADFGSRKKEVAFLGRVPEKRRAIWQSLGMTPRGVDREIAEMMHRTHMGCDNDAASTLIHAARTALADGWAGSMIGTELSDVIFGTPSPKKSTVNLGVINQEQVNILVHGHNPVVSEMIVAAANDPEMLARAEAAGAGGINVAGLCCTGNEILARRGIPMAGNHLMTELAVVTGAVEAVVVDYQCIMPSLLQAAGCYHTQFITTANKARFSGAVHFDIKPATALAQAKEIVQLAIDGFSKRDPQRVEIPSQPVEIMTGFSNEAILNALGGSLTPLLEAIKAGKIRGVVGIVGCNNPKFQQDSGNIGLTKALIQRDILVLVTGCVTTAAGKAGLLLPQAIDQAGPGLQEICGALGIPPVVHFGSCVDNSRIIHLCALIAEELGVDISDLPVAASSPEWYSEKAAAIGLYAVASGIYTHLGLPPNILGSPAVTDLALNGLESLVGASFVVEPDPDKAADLLEARIVSKRKALGLPK

SEQ ID NO: 54 - protein, 625 aa, Pseudodesulfovibrio aespoeensis
MAKEPRPVEELSIWEDARAMIRKARKEGIETVHERLDQQTPHCKFCELGTTCRNCTMGPCRISAKAPRGVCGADADVVVARNFGRFVTGGAAGHSDHGRDLIEVLEAIVEGETKDYRITDEPKLRAIAAEIGVATEGRTVMEVARDVMDAFYADFGSRRKSISFLCRVPEKRKAIWAKLGMTPRGVDREIAEMMHRTHMGCDNDAPNTMIHAARTALADGWGGSMIGTELSDVIFGTPTPKMSTANLAVIKKDQVNILVHGHNPVVSEMILAAAREPELIEQAKKLGATGINVAGLCCTGNELLMRQGIPMAGNHLMTELAIITGAVEAVVVDYQCIMPSLVQISGCYHTKFIDTAAKARFTGAIHFDIHPHNAMEQARKIVGLAVQGFVERDPGRVDIPGEPVDIMTGFSNEAVIKALGGSLDPLIQAIASGDIRGAVGIVGCNNPKFKQDSMNVGLAKELIKKDILVLVTGCVTTAAGKAGLLLPSAIDQAGPGLRKICGSLGIPPVLHYGSCVDNARILQLCAALANGLGVDISDLPVGASSPEWYSEKAAAIGLYAVASGIYTHLGHPPNILGSETVTNLAVSGLEDLVGACFVIEPDPVKAAELFDIRIRAKRKGLGLSE

SEQ ID NO: 55 - protein, 625 aa, Desulfovibrio alaskensis
MAKEPRPVEALSIWEDARAMLTKARAEGIETAHERLQQQTPHCKFCELGVSCRNCTMGPCKITPKAPRGVCGADADVIVARNFGRFVAGGSAGHSDHGRDLVEVLESIVEGSTSDYRITDEAKLRRIAAELGVTVEDRSTMDIAAELIDIFYADFGSRKKEVAFLCRVPAKRRELWDRLGMTPRGVDREIAEMMHRTHMGCDNDAPNTLIHAARTALADGWAGSMIGTELSDVIFGTPQPSMSHANLAAIKKDKVNILVHGHNPVVSEMILAAAREPALIAEAEAMGAQGINVAGLCCTGNELLMRQGIPMAGNHLMTELAIVTGAVEAVVVDYQCIMPSLVQIAGCYHTLFIDTAQKARFTGAVHFDIHPHNALEQAREIVRMAVQAYARRDASRVQIPGEPVNIMTGFSNEAVIAALGGSLDPLVQAIAAGDIRGAVGIVGCNNPKFQQDSMNVGLAKELIKKDILVLVTGCVTTAAGKAGLLLPEAAEQAGPGLRKICGALGIPPVLHYGSCVDNSRILQLCAALANALGVDISDLPVGASSPEWYSEKAAAIGLYAVASGIYTHLGHPPHILGSQTVTDLAVSGLEDLVGASFFIEPDPVKAALAFDLRIRAKRKALGLGE

SEQ ID NO: 56 - protein, 629 aa, Desulfovibrio ferrophilus
MAKELKPPQELSIWEDAQKMIEKARRDGVETVWDRYEQQSPHCTFCELGLTCKNCNMGPCRISPKEGGKMQRGVCGADAHVIVARNFGRFVAGGAAGHSDHGRDLIEVLEAIVEGKAPGYQISDEAKLRRVSAELGINVDGRDVMDVARDLMDICYADFGSRKKEVGFTCRVPEKRKEIWRKLGIMPRGVDREIAEMMHRTHMGCDNDAPNTLLHAARTSLADGWAGSMIGTELSDIIFGTPAPSASRANLGVLKADHVNLLVHGHNPVVSEMVLAAAREPEMIAKAKAAGAAGINIGGLCCTGNELLMRQGIPMAGNHLMTELAIVTGAVDAMVVDYQCIMPSLVQTAACYHTKFITTADKAKFTGATHVSFHPDNAVKQARVVVEMAIEAYERRDQGRVEIPCEPVDITTGFSIEALLSALGGTLDPVLDAVKAGKIRGFTAIVGCNNPRIKHDSANVGLAKALIKKDILVLATGCVTTAAGKAGLLMPEGASMAGPGLQEVCGALGIPPVLHMGSCVDNSRVIQLCAAIANALGVDISDLPVWGASPEWYSEKAVAIGLYCVASGIPVQIGTPPHITGSDVVTNLALSGLEDLVGAAFLVEADPEKAADIMDERIKAKRKALGLSE

SEQ ID NO: 57 - protein, 636 aa, Carboxydothermus hydrogenoformans
MAKQNLKSTDRAVQQMLDKAKREGIQTVWDRYEAMKPQCGFGETGLCCRHCLQGPCRINPFGDEPKVGICGATAEVIVARGLDRSIAAGAAGHSGHAKHLAHTLKKAVQGKAASYMIKDRTKLHSIAKRLGIPTEGQKDEDIALEVAKAALADFHEKDTPVLWVTTVLPPSRVKVLSAHGLIPAGIDHEIAEIMHRTSMGCDADAQNLLLGGLRCSLADLAGCYMGTDLADILFGTPAPVVTESNLGVLKADAVNVAVHGHNPVLSDIIVSVSKEMENEARAAGATGINVVGICCTGNEVLMRHGIPACTHSVSQEMAMITGALDAMILDYQCIQPSVATIAECTGTTVITTMEMSKITGATHVNFAEEAAVENAKQILRLAIDTFKRRKGKPVEIPNIKTKVVAGFSTEAIINALSKLNANDPLKPLIDNVVNGNIRGVCLFAGCNNVKVPQDQNFTTIARKLLKQNVLVVATGCGAGALMRHGFMDPANVDELCGDGLKAVLTAIGEANGLGGPLPPVLHMGSCVDNSRAVALVAALANRLGVDLDRLPVVASAAEAMHEKAVAIGTWAVTIGLPTHIGVLPPITGSLPVTQILTSSVKDITGGYFIVELDPETAADKLLAAINERRAGLGLPW

SEQ ID NO: 58 - protein, 655 aa, Desulfofundulus salinum
PMSDLTGNTKTNSLKGGSTETDHSKKSTDPAVQQMLEKAAREGIQTVWDRYQAMRPLCRFGETGLCCRHCLQGPCRINPLGREPRVGICGATADVIVIRGLDRAIAAGAAGHSGHAKHLAHTLKKAAEGRAPDYLVKDKEKLHSVARRLGIVTDGRPENEIALDVARAALADFHEKDTPVTWVTSVLPPDRLEVFSRHGLVPAGIDHEIAEVMHRTSMGCDADASNLLLGGLRCALGDLAGCYMATDLSDILFGTPSPVVTECNLGVLKAGAVNVAVHGHNPVLSDIIVLVAKEMEAEARAAGAQGINVVGICCTGNEVLMRHGISPCTHSVSQEMAIITGALDALVVDYQCIQPAVATVAECLGTTVITTMDMAKITGTTHVDFSEETAVENARQILRLAIESFTRRRGKPVEIPAMKKKVFAGFSVEAILSALGRLDAGDPLKPLIDHLTAGNIRGVCLFAGCNNVKVPQDYNFITTARKLLEQDVLVLASGCSAGALMRHGFMDPASVEELCGGGLKAVLTAIGEANGLGGPLPPVLHVGSCVDNSRAAALAVALAGRLGVDLDRLPVVASAAEAMHEKAVSIGAWAVALGLPTHVGVMLPVSGGPLVHRILTEEVKGLTGGYFILEPDPESATEKLLEAINERRAGLGLHY

SEQ ID NO: 59 - protein, 642 aa, Caldanaerobacter subterraneus
MTKQCKVSLDPAVCEMVEKARRVKVETVWDRYQAMLPQCGFGETGLCCRHCLQGPCRIDPFGGEPKLGICGATADVIVARGLDRAIAAGAASHSGHARHLAHTLKLAAKGKARDYTIKDKAKLRSVAARLGIPTEGRSIAEIALDVAEAALADFHEKDTPVMWAATTVTKKRARLFEEKGLLPKGIDYEVSDIMHRTSYGVDADPVNLLLGGLRCGLADLAGCYMGTDMADILFGTPQPVVTEANMGVLKADAVNVAVHGHNPVLSEVIVAVAKEMEAEAKAAGASGINVVGICCTGNEVLMRHGIPACTHSVSQEMALVTGALDAMVVDYQCIMPSLATVAECMGTKLITTMEIAKIPGAIHIEFSEEKAGEKAREILRLAIETFTRRRGKPVDIPPYKTKVVAGFSVEAIVKALSKLNAEDPLKPLIDQIAAGNIRGVCLFAGCNNVKVPQDQNFTAIARRLLKENVLVLATGCGAGALMRHGFMDPANVGELCGEGLKAVLTAIGEANGLGGPLPPVLHVGSCVDNSRAVALAVAVADRLGVDTDQLPVVASAAEAVAEKAVSIGTYAVALGLPTHVGVMLPVLGGPLVTKVLTDKVKELTGGYFIVDLDPESAAEKLLAAIDERRAALGLSVPGGGRR

SEQ ID NO: 60 - protein, 642 aa, Thermanaeromonas toyohensis
MANPCKVSLDPAVCEMVEKARRVKVETVWDRYQAMLPQCGFGETGLCCRHCLQGPCRIDPFGGEPKTGICGATADVMVARGLDRAIAAGSAAHSGHARHLAHTLKLAAKGKARDYTVKDKAKLRSVAARLGIPTEGRSIAEIALDVAEAALADFHEKDTPVMWVATTVTKKRAKLFAEKGLLPKGIDYEVADIMHRTLYGTDADPVNLLLGGLRCGLADLAGCYMGTDLSDILFGTPQPVVTEANMGVLKADAVNVAVHGHNPVLSDVIVAVAKEMESEAKAAGASGVNVVGICCTGNEVMMRHGIPACTHSVSQEMALVTGALDAVVVDYQCIMPSLATVAECMGTKLITTMEITKIPGAIHIDFSEETAGENARQIIRLAIESFSRRRGKPVDIPQIKTKVVAGFSVEAIINALAKLNAEDPLKPLIDQIVAGNIRGVCLFAGCNNVKVPQDKNFTAIARRLLKENVLVLATGCGAGALMRHGFMDPANVGELCGEGLKAVLTAIGEANGLGGPLPAVLHVGSCVDNSRAVAVAVAVADRLGVDTDQLPVVASAAEAVSEKAISIGTYAVAIGLPTHVGVMLPVVGGPLVTKVLTEKVKDLTGGYFIVDPDPESAAKKLLAAIDERRAALGLSVPGGGRR

SEQ ID NO: 61 - protein, 631 aa, Desulfobulbus sp.
MDKSRDPAVQQMIARAEAQNVTTVWDRYAAMTPQCGFGDTGLCCRHCLQGPCRIDPFGEGPREGICGASADVMVARGLDRAIAAGTAAHSGHARHLAHTLKKMAMGKAEAYAVKEPGKLEAVAARLGIPTEGRQEREIALDVADAALADFHEKDTPVLWAATVVNPERAKVLADLNLVPKGIDHEVSEIMHRTLYGVDADPVNLLLAGLRCGVADLAGCWMGTDLADILFGIPRPTVSSANLGTLRADAVNIALHGHNPVLSEILVGAVGAKEEAARAAGATGINLVGICCTGNEVMMRHGIPACTHSVSQEMAIMTGAVDAMVVDYQCIMPSVVNVAECTGTRIITTMDIAKITGATHVDFSEEAAAAKAHEIIDQAIDQFRRRRNTPVEIPAVKTPVVAGFSVEAIVAALSGIDAEQPLKPLVDSIKSGAIRGVCLFAGCNNVKVPQDRNFVRMARRLLRENVLVVATGCGAGALMRHGFMDPANVEALCGDGLKAVLTAVGLANDLGGPLPPVLHMGSCVDNSRAVALTVAVAQYLGVDTHQLPIVASAAEAVSEKAVSIGVYAVAAGLPTHVGVMLPVLGSRLVTRVLTDKVKDLTGGYFIVDLDPDSAADKLLAAIDERRRGLGLG

SEQ ID NO: 62 - protein, 634 aa, Desulfotomaculum copahuensis
MQSADPAVREMLDEARRRGVETVWDRYAAQLPQCGFGELGVCCRNCMQGPCRVSPFDDGPARGVCGATADTMVARNLVRAIAAGTAAHSGHAKHLAHTLKKSVQGKAPDYPVRDEAKLRAVAARQGLEADGVPAAELAARMAEAALANFSEGEEPLAWTTATVTAGRVQAWEKLGVLPVGIDSIISEIMHRTHLGVDADAVNLLLGGVACAVADYTGCHLGTDLADILFGTPQPVVSEANMGVLKADQVNIALHGHNPVLSEVIVQVARQMEDEAKAAGAAGINLVGVCCTGNEVLMRHGIPPATHSISQELPMLTGALDAMVVDYQCVYPSLVGVAECTGTKIITTMGMARITGATHVDFEEENAAAGARQIIRLAIEAFGARRGRPVQIPQYKSQVVAGFSVEAIVAALSKLDAADPLKPVIDNIVAGNIQGVCLFAGCNNVKVPQDENYLVMARELAARDVLLLATGCGAGAYARHGYLTPEAVGEYAGPGLKAVLTAVGEANGLGGPLPLVLHMGSCVDNSRAVDLAVAVANRLGVDTAQLPVVASAPEFKTEKAVAIGTWAVACGLPTHLGLVPPVLGSATVTGLLTGGIKDLLGGYFIVETDPVKAAEKLFAAIQERRRGLNLATRSW

SEQ ID NO: 63 - protein, 634 aa, Pelotomaculum propionicicum
MRSRDSAVSLMLEAAQKEAIETTWDRLDAQLPQCGFGELGVCCRHCMQGPCRISPFEDGPQRGICGATADTMVARGLVRAIAAGAAAHSGHAKHLAHTLHKWVKGQAPDYNVKNEAKLRAVAARQGISADGLSVRELAAKVVASVFSQFAEGEEPLAWAGATVTTGRVEALAKLGALPVGIDSSIAEMMHRTHLGVDADPVNLLLGGISCAIADYTGCHIGTDLADILFGTPVPVVSEANMGVLKENSVNIAVHGHNPVLSEVIVRIAGEMQDEAKAAGADGIVLVGICCTGNEVLMRHGIPPVTHGVSQELPILTGALDAIVVDYQCVYPSIVEVARCYGTKVITTMSIAKIQGSTHLEFVEEDAAGKAKEIIGVAIEAFKARKGRPVNIPQVKSKVVAGFSVEAIVAALAKLDAADPLKPVIDNIVNGNIQGVVLFAGCNNVKVPQDHNYLVMAEELAKRDVLLLATGCGAGAYARQGLMTPEATEKYAGPGLKIVLTAIGEANGLGGPLPLVLHMGSCVDNTRAVDLTVAIANKLGVDIDKLPVAASAPEFKTEKAVAIGTWAVASGLPVHLGLVPPVLGGPQVTSILTAGIKDLLGGYFIVETDPQKAAEKLFAAIQERRAGLGLGTRQW

SEQ ID NO: 64 - protein, 645 aa, Methylomusa anaerophila
MEAMKAKKAVKATDCAVGEMLEVARKQGIETVWDRYARQIPQCGFGDSGLCCRNCTQGPCRIDPFGDGPAAGVCGITADSMVARNLIRAIAAGAASHSGHAKHLAHVLKKVAAGKLTDYSVKDENKLRAVATRLGIPTEGKSVQELAAAVASAALEEFSEREEPLAWAATTLTSSQVEKLGGLGVLPAGIDSAISEILQRTTLGVDADPVNLLLGGVKGAVADFAGCHLGTDLADILFGTPTPVFSEANMGVLKENAVNIALHGHNPVVSELIVVAARQEDILAQAKSAGAEGINLVGICCTGNEVLMRQGVAPLTHSLSQEMPILTGALDAIVVDYQCVYPSLANIADCYGTKLITTMDIAKIPGAVHMAIEEERGLEQAKEIIKLAVERFKARKGAPTCIPQVKQKVIAGFSTEAIIGALSKLNAADPLKPLIDNIVNGNIQGVVLFAGCNNVKVPQDANFIAIVKELAKRDVLMLATGCGAGALARHGLLTSEATMEYAGPGLKAVFTAIGEANGLGGPLPLVLHVGSCVDNSRPVDIAVALANKLGVDVNQLPVAASAPEFKAEKAVAIGTWAVTLGLPTHLGLVPPVLGSKLVTRVLTQDIKDITGGYFLVETDPLLAAEKIFDALQERRQKLNIGTRRW

SEQ ID NO: 65 - protein, 640 aa, Sporomusa silvacetica
MKSQEPLDSSVKEMLDVAKTSNVSTVWDRYQSQLPQCGFGESGICCRNCLQGPCRISPFGDGPDVGVCGASADTIVARNLIRGIAGGAASHSGHGKHLAHVLKKVAKGEALAYGVKEAAKLNAVAARLGIPTENRSTKEIAGDVAQAALDEFSERETPNAWAATTLTAGRVAKLNELKVMPYGIDSIIAEIMHRTTLGVDADPVNLLLGGIKGAIADFAGCHLGTDLADILFGIPAPSVSEANMGVIKEKAVNIALHGHNPLLSDMIVATSREPEILAEAKAAGAEQGIQLMGICCTGNEVLMRHGVPTVTTAVSQELPILTGAIDAMIVDYQCVYPSLPTVAACFGTPLITTMDIAKIPGATHIEFDEDHASEKAREIIRIAITAFKRRCNKPVHIPPKTSKVVAGFSTETIVGALAKLNADDPLQPLVDNIVNGNIQGVVLFAGCSNIKVGHDQNFVAIAKELIARNILVLATGCGAGAFARNGLLDPQATLDYAGDGLKAVLTAIGEANGLNGPLPPILHMGSCVDNSRPVDIAVALANKLKVDIPQLPVVASAPEYKTEKALAIGTWAATLGLPTHLGLVPPVVGSKLVTRILTQDIKELTGGYFIVEPDPILAAEKIFNAIQERRQNLNISTRRW

SEQ ID NO: 66 - protein, 636 aa, Heliobacillus mobilis
MDTRSVDLAVQEMLEVALQNKIETAWDRLNFQQPQCGFGELGLCCRNCTQGPCRIDPFGDGPRKGVCGADADTLVARNLARSIVAGTASHSGHSKHLAHVLKKWADGKAPDYPVKDERKLLAVAQRHGIEVEGKEIKEIARSVAEVALEDFSERNTPIGFANRTVTKNRAELWNKFGVNPVGIDSIIAEMMHRTTLGVDADANNLLLGSIAAGIADYAGCRMGTDLSDILFGTPAPVISEMNMAVLDPKQVNVALHGHNPVLSDLLVQVSREMREEAKMAGATGINLVGICCTGSEVLMRHGVPSCTHSVSQELPILTGVLDAMVVDYQCVYPSIATLAGCYSTKVITTMSMAKIPGAIHIEIHEEGAWAKGKEILRLAIEAFKERAGRLYQIPRYKEKVVAGFSKEAIVAALAKLNQEEPLQPLIDNIVNGNIQGICLFAGCNNVKVRQDENYIIMAKELAKRNVLLLATGCGAGSLGRLGFMNGEATKAYAGDGMKDVLSAIGEANGLEGPLPPVLHLGSCVDNSRAVDIAVAIAEKLGVDTDMLPVVASAPEYKTEKAFSIGTYAVALGLPTHLGMVPPVAGSKQVVKTLTKDIREITGGYFIVETNPSEAAKKLFLAIQERRAGLGLLTRLW

SEQ ID NO: 67 - protein, 634 aa, Desulfocucumis palustris
MKSRDKAVNQMIKAAGEREVETVWDRYQTQQPQCGFGELGVCCRNCTQGPCRINPYGDGPDHGVCGANADTIVARNLVRAIAAGAAAHSGHARHLAHALKKGLAGKAPAYGLKDEAKLRALAARVGLDPELPAEELVNKFMDMALEQFAEGEGPLKWAQSFLTGGCGETFERLGTMPTGIDNTIVEMLHRTHLGVDADPVNLLLAGVSCAIADYAGSHLATDLADIMFGTPGPVFSQANMGVMKEKSVNIALHGHNPVLSDIIVQEAREMEEEARGAGAEGINLMGVCCTGNEILMRHGVPALTHSISQEMPMLTGALDAMVVDYQCVYPSLAGVAECTGTKLITTMDMAKISGAVHLGMHEENAAEKAREIISVAIEQFRARQGRPVNIPSVSSKVVAGFSVEAIVQALSKLDAADPLKPVIDSLAAGSLQGVVLLAGCNNVKVPQDDSFLVIARELAARDVLLLATGCGAGAYARHGYLTPEATLEYAGPGLKAVLTAIGEANGLGGPLPLVLHMGSCVDNSRAVDLAVAVADKLGVDLNKLPVVASAPEFKTEKAVSIGTYAMAIGLPVHLGVVPPVLGSRLVTSVLTEGVKDLFGGYFIVETDPYKAAGAIFAAIQERRSGLGLPTREWE

SEQ ID NO: 68 - protein, 1015 aa, Escherichia coli
MDVSRRQFFKICAGGMAGTTVAALGFAPKQALAQARNYKLLRAKEIRNTCTYCSVGCGLLMYSLGDGAKNAREAIYHIEGDPDHPVSRGALCPKGAGLLDYVNSENRLRYPEYRAPGSDKWQRISWEEAFSRIAKLMKADRDANFIEKNEQGVTVNRWLSTGMLCASGASNETGMLTQKFARSLGMLAVDNQARVUHGPTVASLAPTFGRGAMTNHWVDIKNANVVMVMGGNAAEAHPVGFRWAMEAKNNNDATLIVVDPRFTRTASVADIYAPIRSGTDITFLSGVLRYLIENNKINAEYVKHYTNASLLVRDDFAFEDGLFSGYDAEKRQYDKSSWNYQLDENGYAKRDETLTHPRCVWNLLKEHVSRYTPDVVENICGTPKADFLKVCEVLASTSAPDRTTTFLYALGWTQHTVGAQNIRTMAMIQLLLGNMGMAGGGVNALRGHSNIQGLTDLGLLSTSLPGYLTLPSEKQVDLQSYLEANTPKATLADQVNYWSNYPKFFVSLMKSFYGDAAQKENNWGYDWLPKWDQTYDVIKYFNMMDEGKVTGYFCQGFNPVASFPDKNKVVSCLSKLKYMVVIDPLVTETSTFWQNHGESNDVDPASIQTEVFRLPSTCFAEEDGSIANSGRWLQWHWKGQDAPGEARNDGEILAGIYHHLRELYQSEGGKGVEPLMKMSWNYKQPHEPQSDEVAKENNGYALEDLYDANGVLIAKKGQLLSSFAHLRDDGTTASSCWIYTGSWTEQGNQMANRDNSDPSGLGNTLGWAWAWPLNRRVLYNRASADINGKPWDPKRMLIQWNGSKWTGNDIPDFGNAAPGTPTGPFIMQPEGMGRLFAINKMAEGPFPEHYEPIETPLGTNPLHPNVVSNPVVRLYEQDALRMGKKEQFPYVGTTYRLTEHFHTWTKHALLNAIAQPEQFVEISETLAAAKGINNGDRVTVSSKRGFIRAVAVVTRRLKPLNVNGQQVETVGIPIHWGFEGVARKGYIANTLTPNVGDANSQTPEYKAFLVNIEKA

SEQ ID NO: 69 - protein, 1015 aa, Shigella flexneri
MDVSRRQFFKICAGGMAGTTVAALGFAPKQALAQARNYKLLRAKEIRNTCTYCSVGCGLLMYSLGDGAKNAREAIYHIEGDPDHPVSRGALCPKGAGLLDYVNSENRLRYPKYRAPGSDKWQRISWEEAFSRIAKLMKADRDANFIEKNEQGVTVNRWLSTGMLCASGASNETGMLTQKFARSLGMLAVDNQARVUHGPTVASLAPTFGRGAMTNHWVDIKNANVVMVMGGNAAEAHPVGFRWAMEAKNNNDATLIVVDPRFTRTASVADIYAPIRSGTDITFLSGILRYLIENNKINAEYVKHYTNASLLVRDDFAFEDGLFSGYDAEKRQYDKSSWNYQFDENGYAKRDETLTHPRCVWNLLKAHVSRYTPDVVENICGTPKADFLKVCEVLASTSAPDRTTTFLYALGWTQHTVGAQNIRTMAMIQLLLGNMGMAGGGVNALRGHSNIQGLTDLGLLSTSLPGYLTLPSEKQVDLQSYLEANTPKATLADQVNYWSNYPKFFVSLMKSFYGDAAQKENNWGYDWLPKWDQTYDVIKYFNMMDEGKVTGYFCQGFNPVASFPDKNKVVSCLSKLKYMVVIDPLVTETSTFWQNHGESNDVDPASIQTEVFRLPSTCFAEEDGSIANSGRWLQWHWKGQDAPGEARNDGEILAGIYHHLRELYQAEGGKGVEPLMKMSWNYKQPHEPQSDEVAKENNGYALEDLYDANGVLIAKKGQLLSSFAHLRDDGTTASSCWIYTGSWTEQGNQMANRDNSDPSGLGNTLGWAWAWPLNRRVLYNRASADINGKPWDPKRMLIQWNGSKWTGNDIPDFGNAAPGTPTGPFIMQPEGMGRLFAINKMAEGPFPEHYEPIETPLGTNPLHPNVVSNPVVRLYEQDALRMGKKEQFPYVGTTYRLTEHFHTWTKHALLNAIAQPEQFVEISETQAAAKGINNGDRVTVSSKRGFIRAVAVVTRRLKPLNVNGQQVETVGIPIHWGFEGVARKGYIANTLTPNVGDANSQTPEYKAFLVNIEKA

SEQ ID NO: 70 - protein, 1015 aa, Enterobacteriaceae
MDVSRRQFFKICAGGMAGTTVAALGFAPKQALAQARNYKLLRAKEIRNTCTYCSVGCGLLMYSLGDGAKNAREAIYHIEGDPDHPVSRGALCPKGAGLLDYVNSENRLRYPEYRAPGSDKWQRISWEEAFSRIAKLMKADRDANFIEKNEQGVTVNRWLSTGMLCASGASNETGMLTQKFARSLGMLAVDNQARVUHGPTVASLAPTFGRGAMTNHWVDIKNANVVMVMGGNAAEAHPVGFRWAMEAKNNNDATLIVVDPRFTRTASVADIYAPIRSGTDITFLSGVLRYLIENDKINAEYVKHYTNASLLVRDDFAFEDGLFSGYDAEKRQYDKSSWNYQFDENGYAKRDETLTHPRCVWNLLKAHVSRYTPDVVENICGTPKADFLKVCEVLASTSAPDRTTTFLYALGWTQHTVGAQNIRTMAMIQLLLGNMGMAGGGVNALRGHSNIQGLTDLGLLSTSLPGYLTLPSEKQVDLQSYLEANTPKATLAGQVNYWSNYPKFFVSLMKSFYGDAAQKENNWGYDWLPKWDQTYDVIKYFNMMDEGKVTGYFCQGFNPVASFPDKNKVVSCLSKLKYMVVIDPLVTETSTFWQNHGESNDVDPASIQTEVFRLPSTCFAEEDGSIANSGRWLQWHWKGQDAPGEARNDGEILAGIYHHLRELYQAEGGKGVEPLMKMSWNYKQPHEPQSDEVAKENNGYALEDLYDANGVLIAKKGQLLSSFAHLRDDGTTASSCWIYTGSWTEQGNQMANRDNSDPSGLGNTLGWAWAWPLNRRVLYNRASADINGKPWDPKRMLIQWNGSKWTGNDIPDFGNAAPGTPTGPFIMQPEGMGRLFAINKMAEGPFPEHYEPIETPLGTNPLHPNVVSNPVVRLYEQDALRMGKKEQFPYVGTTYRLTEHFHTWTKHALLNAIAQPEQFVEISETLAAAKGINNGDRVTVSSKRGFIRAVAVVTRRLKPLNVNGQQVETVGIPIHWGFEGVARKGYIANTLTPNVGDANSQTPEYKAFLVNIEKA

SEQ ID NO: 71 - protein, 1015 aa, Salmonella typhimurium
MDVSRRQFFKICAGGMAGTTVAALGFTPKMALAQARNYKLLRAKEIRNSCTYCSVGCGLLMYSLGDGAKNAKEAIYHIEGDPDHPVSRGALCPKGAGLLDYVHSEDRLRYPEYRAPGSDKWQRISWDDAFTRIAKLMKADRDANFIEKNEQGVTVNRWLSTGMLCASAASNETGMLTQKFARSLGMLAVDNQARVUHGPTVASLAPTFGRGAMTNHWVDIKNANVVMVMGGNAAEAHPVGFRWAMEAKNNNDATLIVVDPRFTRTASVADIYAPIRSGTDITFLSGVLLYLIENNKINAEYVKHYTNASLLVRDDFAFDDGLFSGYDAQKRQYDKSSWNYQFDENGYAKRDETLTHPRCVWNLLKQHVSRYTPDVVENICGTPKADFLKVCEVLASTSVPDRTTTFLYALGWTQHTVGAQNIRTMAMIQLLLGNMGMAGGGVNALRGHSNIQGLTDLGLLSTSLPGYLTLPSEKQADLQTYLAANTPKATLADQVNYWGNYPKFFVSLMKSFYGDAAQQENDWGFAWLPKWDQSYDVIKYFNMMDSGKVTGYFCQGFNPVASFPDKNKVVQSLSKLKYLVVIDPLVTETSTFWQNHGESNDVDPTTIQTEVFRLPSTCFAEEDGSIANSGRWLQWHWKGQDAPGEARNDGEILAGIYHRLREMYRAEGGKGAEPLLKMSWNYKQPDEPHSEEVAKENNGYALEDLYDANGTLLARKGQLLSSFALLRDDGTTSSSCWIYTGSWTEQGNQMSRRDNADPSGLGNTLGWAWAWPLNRRVLYNRASADPQGKPWDPKRMLIQWNGAKWTGNDIPDFNNAAPGSGTNPFIMQPEGLGRLFAIDKMAEGPFPEHYEPMETPLGTNPLHPNVVSNPAARLYEEDALRMGKKEQFPYVGTTYRLTEHFHTWTKHALLNAIAQPEQFVEISETLAAAKGIANGDYVKVSSKRGFIRAVAVVTRRLRTLHVNGQQVETVGIPIHWGFEGVARKGYIANTLTPNVGDANSQTPEYKAFLVNIEKA

SEQ ID NO: 72 - protein, 1015 aa, Citrobacter rodentium
MDVSRRKFFKICAGGMAGTTVAALGFTPKMALAQARNYKLLRAKEIRNTCTYCSVGCGLLMYSLGDGAKNTKEAIYHIEGDPDHPVSRGALCPKGAGLLDYVHSENRLRYPEYRAPGSDKWQRISWDEAFSRIAKLMKADRDANFIEKNEQGVTVNRWLSTGMLCASAASNETGMLTQKFVRSLGMLAVDNQARVUHGPTVASLAPTFGRGAMTNHWVDIKNANVVMVMGGNAAEAHPVGFRWAMEAKNNNDATLIVVDPRFTRTASVADIYAPIRSGTDITFLSGVLLYLIENNKINAEYVKHYTNASLLVRDDFAFEDGLFSGYDADKRQYDKSSWNYQFDENGFARRDETLSHPRCVWNLLKQHVSRYTPEVVENICGTPKADFLKVCEVLASTSAADRTTTFLYALGWTQHTVGAQNIRTMAMIQLLLGNMGMAGGGVNALRGHSNIQGLTDLGLLSTSLPGYLTLPSEKHSDWQSWLNANTPKATQADQVNYWSNYPKFAVSLMKAFYGDDAQKENDWGFDWLPKWDQAYDVIKYFNMMDSGKVTGYICQGFNPVASFPDKNKVVRSLSKLKFMVVIDPLVTETSTFWQNHGESNDVDPASIQTEVFRLPSTCFAEEDGSIANSGRWLQWHWKGQDAPGEARNDGEILAGIYHRLREMYRTEGGKGAEPLLKMRWSYKQPDHPESEEVAKENNGYALADLYDANGVLIAKKGQLLSSFAQLRDDGTTSSSCWIYTGSWTEQGNQMANRDNADPSGLGNTLGWAWAWPLNRRVLYNRASADINGKPWDPKRMLIQWNGAKWTGNDIPDFNTAPPGSKTGPFIMQPEGVGRLFALDKLAEGPFPEHYEPMETPLGTNPLHPNVVSSPVVRIYEDDVLRLGKKDKFPYVGTTYRLTEHFHTWTKHALLNAIAQPEQFVEISETLAAAKGITNGDRVTVSSKRGFIRAVAVVTRRLRTLNVNGQQVETVGIPLHWGFEGVARKGYIANTLTPNVGDSNSQTPEYKAFLVNIEKA

SEQ ID NO: 73 - protein, 1015 aa, Escherichia alba
MDVSRRKFFKICAGGMAGTTVAALGFSPKMALAQSRNYKLLRAKEIRNSCTYCSVGCGLLMYSLGDGAMNAKEAIYHIEGDPDHPVSRGALCPKGAGLLDYVHSENRLRYPEYRAPGSNKWQRISWNEAFDRIAKLMKADRDANFIEKNEQGVTVNRWLSTGMLCASAASNETGMLTQKFARSLGMLAVDNQARVUHGPTVASLAPTFGRGAMTNHWVDIKNANVVMVMGGNAAEAHPVGFRWAMEAKNHNDATLIVVDPRFTRTASVADIYAPIRSGTDITFLSGVLRYLIENNRINAEYVRHYTNAGLLVRDDFTFEDGLFSGYDAEKRQYDKSSWNYQFDENGHALRDDTLTHPRCVWNLLKQHVSRYTPEVVENICGTPKADFLKVCEVLASTSAADRTTTFLYALGWTQHTVGAQNIRTMAMIQLLLGNMGMAGGGVNALRGHSNIQGLTDLGLLSTSLPGYLTLPSEKQTSLQSWLADNTPKATLPDQVNYWSNYPKFAVSLMKAFYGDDARKENDWGFDWLPKWDQTYDVIKYFNMMDSGKVTGYICQGFNPVASFPDKNKVVRSLSKLKYMVVIDPLVTETSTFWQNHGESNDVDPASIQTEVFRLPSTCFAEEDGSIANSGRWLQWHWKGQDAPGEARNDGQILAGLFHRLREMYRTEGGKGAEPLLKMRWDYKQPNHPESEEVAKENNGYALEDLYDANGKLIAKKGQLLSSFAQLRDDGTTASSCWIYTGSWTEQGNQMANRDNADPSGLGNTLGWAWAWPLNRRVLYNRASADINGKPWDPKRMLIQWDGKKWTGNDIPDFNTAAPGSKTGPFIMQPEGLGRLFALNKLAEGPFPEHYEPMETPLGTNPLHPNVISSPVVRLYEEDAVRMGKKEQFPYVGTTYRLTEHFHTWTKHAKLNAIAQPEQFVEISETLAAAKNIANGDRVTVSSKRGFIRAVAVVTRRLRTLHVNGQQVETIGIPLHWGYEGVARKGYIANTLTPNVGDSNSQTPEYKAFLVNIEKA

SEQ ID NO: 74 - protein, 1015 aa, Enterobacteriaceae
MDVSRRKFFKICAGGMAGTTAAALGFAPKMALAQARNFKLLRAKEIRNTCTYCSVGCGLLMYSLGDGAKNAKEAIYHIEGDPDHPVSRGALCPKGAGLLDYVHSENRLRYPQYRAPGSDKWQRISWDEAFNRIARLMKADRDANFIEKNEQGVTVNRWLSTGMLCASAASNETGMLTQKFVRSLGMLAVDNQARVUHGPTVASLAPTFGRGAMTNHWVDIKNANVVVVMGGNAAEAHPVGFRWAMEAKNNNDATLIVVDPRFTRTASVADIYAPIRSGTDITFLSGVLLYLIENNKINAEYVKHYTNASLLVRDDFAFEEGLFSGYDAEKRQYDKSSWNYQFDENGYAKRDKTLSHPRCVWNLLRQHVSRYTPEVVENICGTPKADFLKVCEVLASTSAADRTTTFLYALGWTQHTVGAQNIRTMAMIQLLLGNMGMAGGGVNALRGHSNIQGLTDLGLLSTSLPGYLTLPSDKQSDLQSYLSANTPKATLPDQVNYWSNYPKFFVSLMKSFYGEAAQKENDWGFNWLPKWDQAYDVIKYFNMMDNGNVTGYICQGFNPVASFPDKNKVVRSLSKLKYLVVIDPLVTETSTFWQNHGESNDVDPAAIQTEVFRLPSTCFAEEDGSIANSGRWLQWHWKGQDAPGEARNDGEILAGIYHRLRELYRREGGKGAEPLLKMSWSYKQPDHPESAEVAKENNGYALADLYDQNGALLAKKGQLLNSFALLRDDGSTASSCWIYTGSWTEQGNQMANRDNADPSGLGNTLGWAWAWPLNRRVLYNRASADINGKPWDAKRMLIQWNGSKWVGNDIPDFNTAPPGSNTGPFIMQQEGLGRLFALDKLAEGPFPEHYEPMETPLGTNPLHPKVVSSPVVRLYEEDAIRLGKKDKFPYVGTTYRLTEHFHTWTKHALLNSIAQPEQFVEISEGLAKSKGIANGDWVKVSSKRGFIRAVAVVTRRLRTLNVNGQQVETVGIPLHWGFEGVARKGYIANTLTPNVGDSNSQTPEYKAFLVNIEKA

SEQ ID NO: 75 - protein, 1015 aa, Enterobacteriaceae
MDVSRRGFFKICAGGMAGTTVAVLGFAPKTALAQARNYKLLRAKEIRNTCTYCSVGCGLLMYSMGDGSKNAKSSIFHIEGDPDHPVSRGALCPKGAGLLDYIHSDNRLRYPQYRAPGSDKWQRISWDDAFKRIARLMKDDRDANFVETNDQGVKVNRWLSTGMLCASAASNETGMLTQKFARSLGMLAVDNQARVUHGPTVASLAPTFGRGAMTNHWVDIKNANVVVVMGGNAAEAHPVGFRWAMEAKNNNDATLIVVDPRFTRTASVADIYAPIRSGTDITFLSGVLLYLINNNKIHADYVKHYTNASLLIRDDYSFDDGLFSGYDADKRQYDKTSWNYQFDENGHALRDMTLTHPRCVWNLLKEHVSRYTPDVVENICGTPKADFLKVCEVLASTSAANRAATFLYALGWTQHTVGAQNIRTMAMIQLLLGNMGVAGGGVNALRGHSNIQGLTDIGLLSTSLPGYLTLPQESHHDVETYLAANTPKALLPGQVNYWSNYPKFYVSLMKTFYGDAATRENGWGYDWLPKWDQAYDVLKYFDMMDRGEVTGYFCQGFNPVASFPDKNKIVASLSKLKYMVVVDPLVTETSNFWQNHGEMNDVDPASIQTEVFRLPSTCFAEEDGSIANSGRWLQWHWKGADAPGEAINDGQILAGLYDELRRLYRAEGGKGVEPLMKMSWNYKQPHEPESEEVAKENNGYALADLFDANGNLLVKKGELLDSFAQLRDDGTTASGCWIYAGSWTRKGNQMANRDNADPSGLGNTLGWTWAWPLNRRVLYNRASCDPQGKAWDPHRLLIEWNGEKWVGNDIPDYSAAAPELNAGPFIMQQEGLGRLFALNKLAEGPFPEHYEPIETPIGTNPLHPNVVSSPVARMFKADAARIGDRKEFPYIGTTYRLTEHFHTWTKHARLNAIAQPEQFVEISEALAKSKGISNGDSVKVSSKRGFIRAKAVVTRRLQTFNVNGQQVETVGIPIHWGFEGKTQKGFVANTLTPSVGDANSQTPEYKAFLVNIEKA

SEQ ID NO: 76 - protein, 1015 aa, Erwinia
MDVSRRSFFKICAGGMAGTTVAALGFAPTTALAEARSYKLLRAKETRNTCTYCSVGCGLLMYSLGDGAKNAKASIFHIEGDPDHPVNRGALCPKGAGLLDYIHSESRLRYPEYRAPGSDKWQRISWEDAFERIAKLMKADRDANFISQNKQGTTVNRWLTTGMLCASAASNETGMLTQKFVRSLGMLAVDNQARVUHGPTVASLAPTFGRGAMTNHWVDIKNANVVVVMGGNAAEAHPVGFRWAMEAKNNNDATLIVVDPRFTRTASVADIYAPIRSGTDITFLSGVLHYLITHNKINAEYVKHYTNAPLLVRDDFRFEDGLFSGYDAEKRQYDKSSWNYQLDENGFARRDDTLSHPRCVWNLLKQHVSRYTPDVVENICGTPQADFLKVCEVLASTSAVDRTTTFLYALGWTQHSVGSQNIRTMAMIQLLLGNMGMAGGGVNALRGHSNIQGLTDLGLLSTSLPGYLTLPKDDQHDLESYLAANTPKATLPGQVNYWGNYPKFFVSLMKTFWGDTATAQNQWGYDWLPKWDQAYDVLNYFDMMDRGEVNGYICQGFNPVASFPDKNKVVASLSKLKYMVVIDPLVTETSNFWQNHGESNDVDPSQIQTEVFRLPSTCFAEEDGSIVNSGRWLQWHWKGADAPGEARNDGEILAGIYHRLRTLYREQGGVASEPVLNMTWDYKLPDGPESEEVAKENNGYALADIHDASGKLLVKKGELLDSFAQLRDDGTTAAGCWIWTGSWTRDGNQMARRDNADPSGLGNTLAWAWAWPLNRRVLYNRASADPAGKAWDPKRVLIEWNGKKWVGNDVPDYNTAAPSENVGPFIMQQEGLGRLFALNKLAEGPFPEHYEPFETPLGTNPLHPNVISNPVARLYQADAARLGERKAFPYVGTTYRLTEHFHTWTKHARLNAIAQPEQFVEISEGLAKAKGIANGDTVKVSSKRGFIRAKAVVTRRLRTLEVNGQQVETVGIPLHWGFEGATRKGYLANTLTPSVGDANSQTPEYKAFLVNIEKA

SEQ ID NO: 77 - protein, 1015 aa, Jejubacter calystegiae
MDVSRRSFFKICAGGMAGTTVAALGFAPTTALAEARSYKLLRAKETRNTCTYCSVGCGLLMYSLGDGAKNAKSSIFHIEGDPDHPVNRGALCPKGAGLLDYIHSESRLRYPEYRAPGSDKWQRISWEDAFERIAKLMKADRDANFISQNKQGTTVNRWLTTGMLCASAASNETGMLTQKFVRSLGMLAVDNQARVUHGPTVASLAPTFGRGAMTNHWVDIKNANVVVVMGGNAAEAHPVGFRWAMEAKNNNDATLIVVDPRFTRTASVADIYAPIRSGTDITFLSGVLHYLIAHNKINAEYVKHYTNAPLLVRDDFSFEDGLFSGYDENKRQYDKSSWNYQLDESGFAKRDDTLSHPRCVWNLLKQHVSRYTPEVVENICGTPQADFQKVCEVLASTSAVDRTTTFLYALGWTQHSVGSQNIRTMAMIQLLLGNMGMAGGGVNALRGHSNIQGLTDLGLLSTSLSGYLTLPKEDQHDLESYLTANTPKATLPGQVNYWGNYPKFFVSLMKTFWGDAATAQNQWGYDWLPKWDQAYDVLNYFDRMDRGEVNGYICQGFNPVASFPDKNKVVASLSKLKYMIVIDPLVTETSNFWQNHGESNDVDSSQIQTEVFRLPSTCFAEEDGSIVNSGRWLQWHWKGADAPGEARNDGEILAGIYHRLRTLYREQGGVASEPVLNMTWDYKLPDGPESEEVAKENNGYALADLYDADGKLLVKKGELLDSFAQLRDDGTTASGCWIWTGSWTRNGNQMAKRDNADPSGLGNTLDWAWAWPLNRRVLYNRASADPAGKAWDPKRVLIEWNGSKWVGNDVPDYNTAAPSQNVGPFIMQQEGLGRLFALNKLAEGPFPEHYEPFETPLGTNPLHPAVISNPVARLYKADAARLGERKAFPYVGTTYRLTEHFHTWTKHARLNAIAQPEQFVEISEGLAKAKGIANGDTVKVSSKRGFIRAKAVVTRRLRTLDVNGQQVETVGIPLHWGFEGATRKGYLANTLTPSVGDANSQTPEYKAFLVNIEKA

SEQ ID NO: 78 - protein, 1015 aa, Moellerella wisconsensis
MNLSRRQFFKICAGGMASTTIAALGFMPGLAMANVREYKLLRSKETRNTCTYCSVGCGLLIYSMGDGAMNAKSSIFHIEGDADHPVNRGALCPKGAGLIDYIHSEGRLRYPEYRAPGSNKWQRISWDDAFSRIARLMKDDRDTNFIEKNSQGTSVNRWLSTGMLCASAASNETGILTQKFTRSLGMLAVDNQARVUHGPTVASLAPTFGRGAMTNHWVDIKNANVIMVMGGNAAEAHPVGFRWAMEAKNNNDATLIVVDPRFTRTASVADHYVPIRSGTDITFLSGVLRYLIENNKINVEYVKHYTNASLLIRDDFSFDDGLFSGYDAEKRQYDKTSWNYQFDENGFAKRDDSWQHPRCVWNLLKQHISRYTPEIVENICGTSQADFLKVCEILATTSSADRTTTFLYALGWTQHTVGSQNIRTMAMIQLLLGNMGMAGGGINALRGHSNIQGLTDLGLLSTSLPGYLSLPSEKQTNIESYLQANTPVATLPNQVNYWSNYPKFFVSLMKSFYGDAAQKDNQWGFDWLPKWDQSYDVIKYFDMMSKGQVTGYICQGFNPVASFPDKNKVVSCLSKLKYLVIIDPLVTETSTFWQHHGEMNDVDPATIQTEVFRLPSTCFAEEDGSIANSGRWLQWHWKAADAPGEARNDGQILAGLLDKIRQLYASEGGKGTEPLMRMRWDYKQPFHPESEEMAKENNGIALVDLYDADGNLQAKKGQLLNSFALLRDDGSTASSCWIYSGCWTEQGNQMAKRDNSDPSGLGNTLGWAWAWPLNRRIIYNRASADPQGKPWDKNRVVIEWKGNKWVGNDVPDFNTSAPDVGTSPFIMQPEGLGRLFAIDKMAEGPFPEHYEPVETPLGTNPLHPNVVSNPAARIFLEDQQRLGNHQDYPYVGTTYRLTEHFHTWTKHARLNAIAQPEQFVEISQTLAQQKGINNGDKVTVSSKRGFIRAIAVVTARLQALHVDGKSIETIGIPIHWGFEGQAQKGFIANTLTPSVGDANSQTPEYKAFLVNIEKA

(19) United States
(12) Patent Application Publication
(10) Pub. No.: US 2026/0043056 A1
(43) Pub. Date: Feb. 12, 2026

(54) REDUCTION OF NITROGEN-CONTAINING GROUPS IN A TARGET COMPOUND USING A BIOCATALYST

(71) Applicant: OXFORD UNIVERSITY INNOVATION LIMITED, Oxford (GB)

(72) Inventors: Kylie VINCENT, Oxford ( Oxfordshire) (GB); Holly REEVE, Begbroke (Oxfordshire) (GB); Jack ROWBOTHAM, Manchester (GB); Sarah CLEARY, Begbroke (Oxfordshire) (GB); Tim SUDMEIER, Oxford ( Oxfordshire) (GB)

(21) Appl. No.: 18/863,983

(22) PCT Filed: May 12, 2023
(86) PCT No.: PCT/GB2023/051250

§ 371 Date: Nov. 7, 2024

(30) Foreign Application Priority Data
May 12, 2022 (GB) 2206982.7

(51) Int. Cl. C12P 13/00; C12N 9/06; C12N 11/14

(52) U.S. Cl. CPC C12P 13/001; C12N 9/0012; C12N 11/14; C12Y 102/07004; C12Y 117/01

(57) ABSTRACT

Provided herein is a method of producing a functional group in a target compound using a biocatalyst as described in more detail herein. Also provided are related systems and compositions.

### CROSS-REFERENCE TO RELATED APPLICATION

The present application is a § 371 national phase of International Application No. PCT/GB2023/051250, filed on May 12, 2023, which claims the benefits of United Kingdom Application No. 2206982.7, filed on May 12, 2022, which applications are incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML, “N422279US.xml”, created on Oct. 15, 2025, and having a size of 113,424 bytes. The contents of the Sequence listing XML are incorporated herein by reference in their entirety.

### FIELD OF THE INVENTION

The present invention relates to methods of reducing a functional group in a target compound using a biocatalyst. In particular, the invention relates to methods of reducing a nitrogen-containing functional group in a target compound using a biocatalyst supported on a support material. The invention further relates to systems and apparatuses for conducting such methods.

### BACKGROUND

Chemical manufacturing processes are typically associated with many environmental concerns. The reagents such as catalysts used are often non-renewable and/or toxic. Extreme operating conditions are typically required, such as elevated temperatures and pressures, with the provision of such conditions being energy inefficient. Toxic solvents are often needed in order to achieve satisfactory yields. Furthermore, the reagents used are often non-selective requiring complex synthetic strategies in order to selectively process only desired functional groups within molecules.

The non-selectivity of conventional processes is particularly problematic in the field of fine chemical manufacture, including in the synthesis of pharmaceuticals, where it is particularly important to minimise production of impurities. Such impurities typically need to be removed through purification strategies, but such purification methods are costly and typically inefficient. Accordingly, the generation of unwanted byproducts in chemical reactions increases financial costs and leads to environmental damage as the chemicals used in purification processes are typically also damaging.

One key class of chemical reaction which is particularly important in synthesis is the reduction of nitrogen-containing functional groups such as nitro (—NO₂) groups, azide (—N₃) groups, nitrile (—CN) groups and the like. Whilst the desired chemical outcome of such reactions is typically formation of an amine (—NH₃) or quaternary ammonium (—NH₄⁺) group, known methods of carrying out such reactions are typically inefficient and lead to significant byproducts, and/or employ expensive non-renewable reagents and environmentally-damaging reaction conditions.

For example, some strategies for the industrial reduction of nitro groups to amine groups rely on the use of precious metal catalysts, for example palladium (Pd). However, such catalysts are extremely expensive, are non-renewable, are associated with significant environmental impact during their production, and have activity that is typically reduced in the presence of common contaminants in feedstocks. For example, carbon monoxide (CO) is a common contaminant in hydrogen sources obtained from natural gas, which are often used as a reductant in such reactions, meaning that high purity hydrogen (with its own financial and environmental implications) is typically required in such reactions. Furthermore, the reaction conditions necessary when using such catalysts are typically harsh, employing elevated temperatures and/or hazardous organic solvents. Furthermore, such reactions often generate significant side-products e.g. arising from non-specific reduction, incomplete reduction, and catalysis of unwanted side-reactions such as bond rearrangements.

In attempts to avoid the problems associated with the use of precious metal catalysts for nitrogen-group reduction, some recent efforts have focused on the use of biological catalysts such as enzymes. Enzymatic processing of chemical reagents offers advantages compared to traditional chemical processing methods. Enzymes are renewable and biodegradable, and thus overcome environmental issues regarding the production and disposal of chemical catalysts. Enzymes are typically non-hazardous and nontoxic, thus addressing safety concerns associated with chemical catalysts. Enzymes typically operate under moderate temperatures and at atmospheric pressure, thus reducing the energy demands associated with conventional chemical processing. Enzymes are also typically highly selective as regards their chemical substrate, and approaches such as rational enzyme engineering and directed mutagenesis continue to expand the range of reactions that can be undertaken. Enzymatic catalysis thus provides many advantages compared to conventional chemical approaches.

These advantages have led to investigations into the industrial use of enzymes which can catalyse nitro-group reductions (e.g. nitroreductase enzymes). Such enzymes have active sites which are capable of catalysing the reduction of nitro-groups. However, whilst the use of such enzymes does address at least in part issues arising with precious metal catalysts as set out above, significant problems remain.

For example, enzyme catalysts such as nitroreductases are typically used as homogeneous catalysts in solution with the reactant (and typically also the product) molecules. However, this creates problems associated with the need to purify the enzyme from the final product mix. The need to separate the solution-phase enzyme from the final product increases costs and processing time, may involve separation techniques which use harsh chemical conditions, and may even result in the product undergoing further unwanted reactions within the purification workstream.

Furthermore, the nitroreductase enzymes that have been investigated for use in the reduction of nitrogen-containing functional groups typically require supporting cofactors in order for their catalytic cycle to operate. As explained below, the need to recycle the cofactors required in their catalytic cycle is a particular difficulty with the use of such enzymes.

Cofactors are non-protein chemical compounds that play an essential role in many enzyme catalysed biochemical reactions, and which typically act to transfer chemical groups between enzymes. Cofactors are also sometimes known as “co-substrates” reflecting their processing by an enzyme in the course of its catalysing of its primary reaction. By way of illustration, a redox enzyme which catalyses an oxidation reaction of a reagent to produce a product may couple that oxidation to the reduction of a cofactor as an electron sink. In this case, the overall reaction catalysed by the enzyme may be represented as:
reduced reagent+oxidised cofactor→oxidised product+reduced cofactor

Similarly, enzymes which catalyse the reduction of a reagent to produce a product typically couple that reduction with the oxidation of a cofactor as a source of electrons or reducing equivalents such as hydride ions:
oxidised reagent+reduced cofactor→reduced product+oxidised cofactor

Biological use of cofactors is not limited to simple redox reactions as represented above but is also involved in more complex reactions such as atom insertion reactions, rearrangement reactions, etc.

Key biological cofactors include nicotinamide adenine dinucleotide (NAD), nicotinamide adenine dinucleotide phosphate (NADP), and flavins such as flavin mononucleotide (FMN), flavin adenine dinucleotide (FAD), and riboflavin.

Whilst enzymes which rely on cofactor regeneration are widely known, their use in industry has been limited by the difficulty in providing sufficient cofactor for the enzyme to use. One option is to provide the cofactor in superstoichiometric quantities relative to the reagent at issue. However, the high cost and typically low stability of reduced cofactor molecules means that this is not a viable approach. Other approaches that have been considered include the use of systems for regenerating cofactor molecules in their desired form (i.e., recycling the cofactor molecule). However, conventional cofactor regeneration systems are problematic. For example, current industrial practices for enzymatic NAD(P)H recycling rely on a superstoichiometric quantity of a carbon-based (organic) sacrificial reductant such as glucose or isopropanol. However, this leads to additional cost, generates waste products, and requires additional downstream processing steps of the desired product, and is atom inefficient. The industrial exploitation of flavin-utilising enzymes has been almost wholly prevented by a lack of suitable means for recycling the flavin cofactor. Electrochemical reduction of oxidised flavin cofactors has been proposed, but such systems are difficult to incorporate into industrially relevant contexts, involve the use of costly materials such as precious metals and highly-processed carbon materials as the required electrode materials, and are prone to generation of unwanted side-reaction products.

Accordingly, there is a pressing need for improved methods of catalysing the reduction of reducible functional groups, in particular nitrogen-containing functional groups. In particular, there is a need for methods that avoid the requirement for expensive or dangerously reactive chemical reagents; that are atom efficient; that avoid difficulties associated with electrochemical processing of reagents; that do not rely on the use of expensive sacrificial organic reductants; that are selective; that lead to complete rather than only partial reduction; that avoid the generation of by-products; and/or which do not rely on complex cofactor regeneration methods. The present invention aims to address some or all of these problems.

### SUMMARY OF THE INVENTION

In seeking to address the above problems, the present inventors have surprisingly found that target compounds containing reducible nitrogen-containing functional groups can be cleanly and efficiently reduced by the use of a biocatalyst comprising an oxidoreductase enzyme such as a hydrogenase enzyme supported on a support material such as carbon, in the presence of a molecular reductant such as hydrogen. Surprisingly, the inventors have found that by contacting the target compound with such a catalyst, the molecular reductant can be oxidised by the oxidoreductase enzyme and the nitrogen-containing functional group can be reduced at the support material.

As explained in more detail herein, the reduction is clean, and atom efficient. The reactions take place under mild conditions and avoid or minimise the use of toxic reagents such as organic solvents, which as explained above are associated with the use of precious metal catalysts. Side reactions such as rearrangements can be avoided and there is no need for expensive sacrificial organic reductants. The enzymes used typically do not rely on cofactors which need to be regenerated (although such enzymes are not necessarily excluded). The catalyst is heterogeneous, which leads to easier purification of the product from the reaction mixture. In particular, the reactions typically lead to much more complete reduction (up to 100% complete reduction of the nitrogen-containing functional group) compared to the use of similar enzymes in solution.

Accordingly, provided herein is a method of reducing a nitrogen-containing functional group in a target compound, comprising contacting the target compound with a biocatalyst comprising an oxidoreductase enzyme or a functional fragment or derivative thereof supported on a support material, in the presence of a molecular reductant for oxidation by the oxidoreductase enzyme or functional fragment or derivative thereof, under conditions such that:
the molecular reductant is oxidised by the oxidoreductase enzyme or functional fragment or derivative thereof; and
the nitrogen-containing functional group is reduced at the support material.

Preferably, said method comprises contacting the target compound with the support material.

Preferably, reduction of the nitrogen-containing functional group comprises direct electron transfer from the support material to the target compound. Preferably, said method comprises reducing the nitrogen-containing functional group to form an amine group or a quaternary ammonium group.

Preferably, the oxidoreductase enzyme or functional fragment or derivative thereof is in electronic contact with the support material. Preferably, the oxidoreductase enzyme or functional fragment or derivative transfers electrons to the support material via an intramolecular electronically-conducting pathway. Preferably, said intramolecular electronically-conducting pathway comprises a series of [FeS] clusters.

Preferably, the molecular reductant is selected from hydrogen, carbon monoxide, formate, isotopes thereof, and mixtures thereof. Preferably the molecular reductant comprises or consists of hydrogen or an isotope thereof.

Preferably, in one embodiment, the supported biocatalyst comprises a hydrogenase enzyme or a functional fragment or derivative thereof. Preferably the hydrogenase is selected from or comprises the amino acid sequence of any one or more of SEQ ID NOs: 1 to 46, or an amino acid sequence having at least 60% homology therewith; or a functional fragment, derivative or variant thereof.

Preferably, in another embodiment, the supported biocatalyst comprises a carbon monoxide dehydrogenase enzyme or a functional fragment or derivative thereof. Preferably the carbon monoxide dehydrogenase is selected from or comprises any one or more of SEQ ID NOs: 47 to 67, or an amino acid sequence having at least 60% homology therewith; or a functional fragment, derivative or variant thereof.

Preferably, in yet another embodiment, the supported biocatalyst comprises a formate dehydrogenase enzyme or a functional fragment or derivative thereof. Preferably, the formate dehydrogenase is selected from or comprises any one or more of SEQ ID NOs: 68 to 78, or an amino acid sequence having at least 60% homology therewith; or a functional fragment, derivative or variant thereof.

Preferably, the oxidoreductase enzyme or functional fragment or derivative thereof is immobilised on the support material. Preferably, the support material is electronically conductive or semi-conductive.

Preferably, the support material comprises carbon, a metal or metal alloy, a metal oxide or mixed metal oxide, a metal hydroxide, a metal chalcogenide, a semi-conducting material, or an electronically-conductive polymer, or mixtures thereof.

Preferably, the support material comprises or consists of a carbon material.

Preferably, the carbon material comprises graphite, carbon nanotube(s), carbon black, activated carbon, carbon nanopowder, vitreous carbon, carbon fibre(s), carbon cloth, carbon felt, carbon paper, graphene, highly oriented pyrolytic graphite, pyrolytic graphite, doped or surface-modified carbon or doped diamond. Preferably, the carbon material comprises:
doped graphene, wherein said graphene is doped with one or more dopants selected from nitrogen, boron, sulphur, oxygen, silicon, lanthanide elements and transition-metals;
doped carbon nanotube(s), wherein said carbon nanotube(s) are doped with one or more dopants selected from nitrogen, boron, sulphur, oxygen, silicon, lanthanide elements and transition-metals;
doped diamond, wherein said diamond is doped with one or more dopants selected from nitrogen, boron, sulphur, oxygen and silicon;
doped carbon black, wherein said carbon black is doped with one or more dopants selected from nitrogen, boron, sulphur, oxygen, silicon, lanthanide elements and transition-metals; and/or
doped activated carbon, wherein said activated carbon is doped with one or more dopants selected from nitrogen, boron, sulphur, oxygen, silicon, lanthanide elements and transition-metals;
   and/or wherein said carbon material comprises or is modified to comprise carboxylic acid surface groups.

Preferably, the nitrogen-containing functional group is a nitro, an azide, a hydroxylamine, a nitroso, a nitrile, a diazo, a diazonium, an isocyanide, an isothiocyanate, an isocyanate, a hydrazone, a hydrazine, an amidine, an azo, or a guanidine group.

Preferably, the target compound is a nitroaromatic compound. Preferably, the nitroaromatic compound comprises a hydrocarbyl aromatic group or a heteroaromatic group substituted with to a nitro group, wherein said hydrocarbyl aromatic group or a heteroaromatic group is optionally further substituted.

Preferably, the biocatalyst does not comprise a oxidoreductase enzyme or functional fragment or derivative thereof comprising an active site capable of catalysing the enzymatic reduction of the nitrogen-containing functional group. Preferably, the biocatalyst does not comprise a nitro reductase enzyme. Preferably, said method does not comprise transfer of electrons to the target compound via one or more cofactors.

Also provided herein is a system, comprising:
i) a biocatalyst comprising an oxidoreductase enzyme or a functional fragment or derivative thereof supported on a support material;
ii) a molecular reductant; and
iii) a target compound comprising a reducible nitrogen-containing functional group;
wherein the system is configured such that, in use, (a) the molecular reductant is oxidised by the oxidoreductase enzyme or functional fragment or derivative thereof; and (b) the nitrogen-containing functional group is reduced at the support material.

Preferably, in said system, the oxidoreductase enzyme or functional fragment or derivative transfers electrons to the support material and the reduction of the nitrogen-containing functional group comprises direct electron transfer from the support material to the target compound.

Preferably, in said system, the oxidoreductase enzyme or a functional fragment or derivative thereof is as defined herein; and/or the support material is as defined herein; and/or the molecular reductant is as defined herein; and/or the target compound and/or nitrogen-containing functional group are as defined herein; and/or the biocatalyst is as defined herein.

### BRIEF DESCRIPTION OF THE FIGURES

FIG. 1: Cyclic voltammograms of a PGE electrode in 50 mM tris-HCl pH 8 (black in original colour image) and in 50 mM tris-HCl pH 8 with 1 vol % DMSO and 1 mM nitrobenzene (red in original colour image) at a rotation rate of 0 rpm obtained with a scan rate of 100 mV s⁻¹. The reductive wave in the presence of nitrobenzene begins at a potential more positive than the hydrogen couple, confirming the potential for reduction of nitrobenzene using hydrogen as a molecular reductant in accordance with the disclosed methods.

FIG. 2: Cyclic voltammograms of a BP-modified PGE electrode in 50 mM tris-HCl pH 8 with 2 vol % DMSO and 1 mM nitrobenzene at rotation rate of 0 rpm (red in original colour image) and 2000 rpm (blue in original colour image) obtained with a scan rate of 100 mV s⁻¹. The reductive wave in the presence of nitrobenzene begins at a potential more positive than the hydrogen couple, confirming the potential for reduction of nitrobenzene using hydrogen as a molecular reductant in accordance with the disclosed methods.

FIG. 3: IR difference spectrum of 2 mM nitrobenzene in 50 mM tris-HCl pH 8 with 1 vol % DMSO subtracting the spectrum of pure 50 mM tris-HCl pH 8 at open-circuit potential. The dotted lines indicate characteristic peaks for nitro groups.

FIG. 4: IR difference spectrum of 2 mM nitrobenzene in 50 mM tris-HCl pH 8 with 1 vol % DMSO at −0.47V vs SHE applied to a BP-modified activated carbon paper working electrode subtracting the spectrum at open-circuit potential. As can be seen, the dotted lines indicate the characteristic peaks for nitro groups are decreasing, showing reduction at the hydrogen potential.

FIG. 5: Cyclic voltammograms of a BP-modified PGE electrode in 100 mM sodium phosphate pH 6 (black in original colour image) and 1 mM nitrophenol (blue in original colour image) at a rotation rate of 0 rpm obtained with a scan rate of (A) 100 mV s⁻¹ or (B) 10 mV s⁻¹. The reductive wave in the presence of nitrophenol begins at a potential more positive than the hydrogen couple, confirming the possibility of reduction of nitrophenol using hydrogen as a molecular reductant in accordance with the disclosed methods.

FIG. 6: Cyclic voltammograms of a BP-modified activated carbon paper electrode in 50 mM tris-HCl pH 8 (light grey) with 1 vol % DMSO and 5 mM 2-azido-1-phenylethanone (black) obtained with a scan rate of 100 mV s⁻¹ in a spectroelectrochemical cell. The reductive wave in the presence of the azido-compound begins at a potential more positive than the hydrogen couple, confirming the possibility for reduction of azide using hydrogen as a molecular reductant in accordance with the disclosed methods.

FIG. 7: IR difference spectrum of 5 mM 2-azido-1-phenylethanone in 50 mM tris-HCl pH 8 with 1 vol % DMSO at open-circuit potential. The dotted lines indicate characteristic peaks for azide groups.

FIG. 8: IR difference spectra of 5 mM 2-azido-1phenylethanone in 50 mM tris-HCl pH 8 with 1 vol % DMSO at different potentials (vs SHE) applied to a BP-modified activated carbon paper working electrode. As can be seen, the dotted lines indicate the characteristic peaks for azide groups are decreasing, showing reduction at the hydrogen potential.

FIG. 9: Cyclic voltammograms of a PGE electrode modified with BP200 in 100 mM sodium phosphate pH 6 with 1 vol % DMSO and 1 mM tetracyanobenzene at a rotation rate of 0 rpm obtained with a scan rate of 10 mV s⁻¹. The reductive wave in the presence of the cyano-compound begins at a potential more positive than the hydrogen couple, confirming the possibility for reduction of cyano groups using hydrogen as a molecular reductant in accordance with the disclosed methods.

FIG. 10: ¹H NMR spectra corresponding with Table 1 (see Example 1). Compound standards (A, B, C) and reaction mixtures in H₂O/D₂O, un with water suppression. Compound A was reduced to C using reported conditions,^{Ref 4} resulting in a mixture of B and C in this case. A: expanded spectrum. B: zoomed-in spectrum.

FIG. 11: A: ¹H NMR spectrum to correspond with Scheme 1(i) (see Example 2). This example was taken from a reaction that used phosphate buffer (50 mM, pH 7.0) at room temperature under 1 bar H₂. The Hyd1/C was formed from 19 μg Hyd1 immobilised onto 25 μg BP2000. The compound standards (commercially available) and reaction mixtures in H₂O/D₂O were run with water suppression. B: ¹H NMR spectrum to correspond with Scheme 1(ii) (see Example 2). The top shows the spectrum of the authentic product standard (commercially available) and the bottom shows the spectrum obtained from the reaction mixture. Both spectra were in H₂O/D₂O and were run with water suppression.

FIG. 12: HPLC traces detected at 220 nm to show compound standards of 2-azido-1-phenylethanone and 2-amino-1-phenylethanone that had been subjected to Boc-derivitisation for 12 h at room temperature: amine (1 equiv), Boc₂O (1.5 equiv) and NaHCO₃ (2.5 equiv) in 1:1 methanol/water (1 mL). A 0.2 mL portion of this mixture was extracted into hexanes (0.6 mL) that contained 2 mM toluene as an internal standard. This solution was filtered using 22 μm syringe filters (Fischer Scientific, nylon membrane, 13 nm diameter). Peak areas were determined by integration of the peaks shown in the chromatograms, and calibration curves were used to determine the correlation between compound concentration and peak area. Conversions listed in Table 2 (Example 4) were determined based on concentrations of the azide and amine calculated using this method.

FIG. 13: ¹H NMR spectra corresponding with Scheme 3 (Example 6). Cinnamyl azide (bottom spectrum) was synthesised from cinnamyl alcohol using a published procedure.⁵ The middle spectrum used 10 wt % Pd/C in place of Hyd1/C.

FIG. 14: Cyclic voltammograms of a BP-modified PGE electrode in 100 mM sodium phosphate pH 6 and 1 mM nitrohexane at a rotation rate of 0 rpm obtained with a scan rate of 10 mV s⁻¹. Thermodynamic potential of the hydrogen couple is shown as red dotted line in original colour image and the overpotential at which Hyd1 is operating is shown as blue dotted line in original colour image. Data discussed in Example 10.

### DETAILED DESCRIPTION OF THE INVENTION

### Methods of the Invention

As discussed above, provided herein is a method of reducing a functional group in a target compound, comprising contacting the target compound with a biocatalyst comprising an oxidoreductase enzyme or a functional fragment or derivative thereof supported on a support material, in the presence of a molecular reductant for oxidation by the oxidoreductase enzyme or functional fragment or derivative thereof, under conditions such that:
the molecular reductant is oxidised by the oxidoreductase enzyme or functional fragment or derivative thereof; and
the functional group is reduced at the support material.

The functional group is a reducible functional group. Typically, the functional group is a nitrogen-containing functional group. Suitable nitrogen-containing functional groups are discussed below.

Accordingly, provided herein is a method of reducing a nitrogen-containing functional group in a target compound, comprising contacting the target compound with a biocatalyst comprising an oxidoreductase enzyme or a functional fragment or derivative thereof supported on a support material, in the presence of a molecular reductant for oxidation by the oxidoreductase enzyme or functional fragment or derivative thereof, under conditions such that:
the molecular reductant is oxidised by the oxidoreductase enzyme or functional fragment or derivative thereof; and
the nitrogen-containing functional group is reduced at the support material.

The methods provided herein comprise reducing the functional group of the target compound at the support material. Thus, the methods typically comprise contacting the target compound with the support material. Suitable support materials are described in more detail herein.

Typically, the methods provided herein involve the direct transfer of electrons and/or hydride ions from the support material to the target compound. More typically, the methods provided herein involve the direct transfer of electrons from the support material to the target compound. Typically, the methods involve the direct transfer of electrons from the support material to the reducible functional group of the target compound. In some embodiments the direct transfer of electrons comprises electron tunnelling from the support material to an atom of the functional group of the target compound. In some embodiments the reducible functional group of the target compound contacts the surface of the support material. Direct electron transfer from the support material to the functional group of the target compound thus occurs at the solvent-accessible surface of the support material, i.e. at the interface between the support material and the solvent (e.g. an aqueous reaction solvent). Direct electron transfer from the support material to the functional group of the target compound typically does not comprise contacting the target compound with a reduction enzyme (e.g. nitroreductase) on the support material. In other words, the disclosed methods typically do not comprise contacting the target compound or the reducible functional group thereof with an enzyme immobilized on the support material. As such, the support material can be considered as providing an “active site” (using terminology analogous to enzymology) for reduction of the reducible functional group.

The electron transfer from the support material to the functional group is typically not mediated by an enzyme or cofactor thereof. Cofactors are described above. In other words, the disclosed method typically does not comprise transfer of electrons to the target compound via one or more cofactors.

In particular, the electron transfer from the support material to the functional group is typically not catalysed by an enzyme for reducing the functional group of the target compound. Thus, for example, the supported biocatalyst typically does not comprise a reduction enzyme capable of catalysing the reduction (e.g. the complete reduction) of the functional group. In other words, the supported biocatalyst typically does not comprise a oxidoreductase enzyme or functional fragment or derivative thereof comprising an active site (e.g. a substrate-binding active site) capable of catalysing (e.g. natively catalysing) the enzymatic reduction of the nitrogen-containing functional group, such as a nitroreductase.

Thus, it will be clear that, as used herein, direct electron transfer from the support material to the functional group does not comprise the enzymatic reduction of the functional group e.g. using a nitroreductase, cytochrome P450 monooxygenase, nitrile reductase, azoreductase, etc. Accordingly, typically the biocatalyst does not comprise a nitroreductase. Similarly, the biocatalyst typically does not comprise an azoreductase or a nitrile-reductase or other enzyme comprising an active site (e.g. a substrate-binding active site) capable of reducing the reducible functional group. In some embodiments, the supported biocatalyst consists of a oxidoreductase enzyme as described herein supported on the support.

It is thus important to distinguish between the oxidoreductase enzyme which catalyses the oxidation of the molecular reductant, and a reduction enzyme which typically has an active site (e.g. a substrate-binding active site) for catalysing the reduction of the reducible functional group of the target compound. As used herein, an oxidoreductase enzyme for catalysing the oxidation of the molecular reductant is not an example of a reduction enzyme for reducing the reducible functional group of the target compound. For example, the oxidoreductase enzyme for catalysing the oxidation of the molecular reductant typically does not comprise an active site (e.g. a substrate-binding active site) capable (e.g. natively capable) of accommodating a target compound or a reducible functional group thereof. The oxidoreductase enzyme for catalysing the oxidation of the molecular reductant may operate only at a potential or under reaction conditions incompatible with the enzymatic reduction of the reducible functional group of the target compound by a reducing enzyme such as a nitroreductase, azoreductase or nitrile-reductase. For example, reducing enzymes for reducing reactive functional groups typically require cofactors as described herein (e.g. NADH, NAD(P)H or flavins such as FAD, FMN or riboflavin) in order to mediate electron transfer to their oxidised substrates.

Typically, in the methods herein, no such cofactors are present. Thus, typically the disclosed methods do not comprise use of a cofactor in the reaction medium, e.g. in the reaction solution.

As noted above, the target compound comprises a reducible functional group.

Typically the reducible functional group is a nitrogen-containing functional group. Examples of suitable nitrogen-containing functional groups are described in detail herein. As will be apparent from the discussion herein, such groups include nitro groups (R—NO₂), azide groups (R—N₃), hydroxylamine groups (R—NR′OH), nitroso groups (R—NO), nitrile groups (R—CN), diazo groups (R—CR′═N₂), diazonium groups (R—N₂⁺), isocyanide groups (R—NC); isothiocyanate groups (R—NCS), isocyanate groups (R—N═C═O), hydrazone groups (R—CR′═N—NR′), hydrazine groups (R—NR′—NR′₂), amidine groups (R—C(NR′)NR′₂), azo groups (R—N═N═R′), and guanidine groups (R—NR′—C(NR′)—NR′₂), etc; wherein R represents the remainder of the target compound and each R independently represents a group such as H or hydrocarbyl, e.g, alkyl, e.g. Cie or C₁₋₄ alkyl such as methyl or ethyl; typically H). More typically, the nitrogen-containing functional group is a nitro group (such as an aromatic nitro group); an azide group (such as an aromatic azide group) or a nitrile group (such as an aromatic nitrile group). Still more typically the nitrogen-containing functional group is a nitro group (such as an aromatic nitro group) or an azide group (such as an aromatic azide group); most typically the nitrogen-containing functional group is a nitro group (such as an aromatic nitro group).

Typically, the methods disclosed herein comprise the substantially complete or complete reduction of the reducible functional group. As used herein, the term “complete reduction” means that the reducible functional group (e.g. the nitrogen atom in the reducible functional group) is reduced to the lowest stable oxidation state. For example, the complete reduction of a nitro group corresponds to the 6-electron reduction of the nitro group (R—NO₂) to an amine (R—NH₂) or quaternary ammonium (R—NH₃⁺) group, whereas the partial reduction of a nitro group may result in the formation of for example hydroxylamine (R—NHOH). Thus, for example, when the reducible functional group is a nitro group (R—NO₂), (e.g. an aromatic-nitro group) the method typically comprises reducing the nitro group to an amine or quaternary ammonium (R—NH₃⁺) group (wherein R is the remainder of the target compound). When the reducible functional group is an azide group (R—N₃) (e.g. an aromatic nitrile group) the method typically comprises reducing the azide group to an amine or quaternary ammonium (R—NH₃⁺) group (wherein R is the remainder of the target compound). When the reducible functional group is a nitrile group (R—CN) (e.g. an aromatic nitrile group) the method typically comprises reducing the nitrile group to an amine or quaternary ammonium (e.g. R—CR′2NH₃⁺) group (wherein R is the remainder of the target compound).

The methods may comprise contacting a sample comprising a plurality of target compounds with a biocatalyst in the presence of a molecular reductant, as defined in more detail herein. Typically, at least 50%, e.g. at least 60% such as at least 70%, at least 80%, at least 90%, at least 95%, at least 97%, at least 98%, at least 99%, at least 99.9% or at least 99.99% of the target compounds in the sample may be completely reduced at the nitrogen-containing functional group.

The methods disclosed herein are typically highly selective. Typically, the methods disclosed herein are selective for reduction of a nitrogen-containing functional group even in the presence of a C═C double bond. For example, the methods may comprise contacting a target compound comprising a nitrogen-containing reducible functional group and a C═C double bond with a biocatalyst as described herein. In such embodiments, typically the nitrogen-containing reducible functional group is reduced and the C═C double bond is not reduced. This is described in more detail in the examples. This may represent a particular advantage of the disclosed methods, as such selective reduction is typically not possible with precious metal catalysts such as palladium.

### Reductant

As will be apparent from the above discussion, the disclosed methods comprise the oxidation of a molecular reductant by the oxidoreductase enzyme or functional fragment or derivative thereof. Suitable enzymes are described in more detail herein.

Typically, the molecular reductant is selected from hydrogen (H₂), carbon monoxide (CO), formate (either deprotonated as CH(O)O⁻, or protonated HC(O)OH which is also known as formic acid), isotopes thereof, and mixtures thereof. Particular benefits may arise from the use of such reductants as the products of their oxidation may be easily removed from the reaction mixture and/or do not need removing. Thus, the protons formed by oxidation of molecular hydrogen typically do not need to be removed from the reaction mixture. The carbon dioxide which results from CO or formate oxidation can be readily removed e.g. as a gas. Thus reactions which use such reductants can be clean and avoid the need for extensive purification. Typically, the molecular reductant is selected from hydrogen and carbon monoxide, isotopes thereof, and mixtures thereof. More typically, the molecular reductant is hydrogen (i.e. molecular hydrogen) or an isotope thereof.

In some embodiments, the reductant used in the disclosed methods is determined by the oxidoreductase enzyme comprised in the biocatalyst. In other embodiments, the oxidoreductase enzyme comprised in the biocatalyst is determined by the reductant used. In view of the present disclosure, it will be straight forward for those of skill in the art to select compatible oxidoreductase enzymes/reductant pairs. Exemplary combinations of

| Oxidoreductase enzyme | Molecular reductant |
|---|---|
| Hydrogenase, or a functional fragment or | Molecular hydrogen, or an |
| derivative thereof | isotope thereof |
| Carbon monoxide dehydrogenase, or a | Carbon monoxide, or an |
| functional fragment or derivative thereof | isotope thereof |
| Formate dehydrogenase, or a functional | Formate, or an isotope |
| fragment or derivative thereof | thereof |

Mixtures of oxidoreductase enzymes and corresponding molecular reductants can be used. For example, the biocatalyst may comprise a mixture of one or more hydrogenases (or a functional fragment or derivative thereof) and one or more carbon monoxide dehydrogenases (or a functional fragment or derivative thereof), and the molecular reductant may comprise a mixture of molecular hydrogen (or an isotope thereof) and carbon monoxide (or an isotope thereof). The biocatalyst may comprise a mixture of one or more hydrogenases (or a functional fragment or derivative thereof) and one or more formate dehydrogenases (or a functional fragment or derivative thereof), and the molecular reductant may comprise a mixture of molecular hydrogen (or an isotope thereof) and formate (or an isotope thereof). The biocatalyst may comprise a mixture of one or more carbon monoxide dehydrogenases (or a functional fragment or derivative thereof) and one or more formate dehydrogenases (or a functional fragment or derivative thereof), and the molecular reductant may comprise a mixture of carbon monoxide (or an isotope thereof) and formate (or an isotope thereof).

When the molecular reductant is hydrogen, suitable isotopes thereof include ¹H₂, ²H₂ and ³H₂. Mixed isotopes (e.g. ¹H₂H and ¹H₃H) are also embraced. Preferably, the hydrogen is ¹H₂. It will be apparent that, as used herein, organic molecules such as glucose, formate, and ethanol, isopropanol, etc, are not sources of molecular hydrogen, although in some embodiments such compounds can be used as the molecular reductant as described in more detail herein.

Typically, when the reductant is molecular hydrogen, the molecular hydrogen is provided in the form of a gas. The gas may be mixed with an aqueous solution in which the biocatalyst and target compound as well as optionally other reaction components are present. At 1 bar H₂ the solubility of H₂ in water is 0.8 mM. In other words, when the reductant is molecular hydrogen provided in the form of molecular hydrogen gas, the concentration of hydrogen is solution (i.e. that under which the oxidoreductase operates) is 0.8 mM hydrogen. Other pressures may also be used. For example, the gas pressure in the reaction vessel may be from 0.01 to about 100 bar, such as from 0.1 to 10 bar, e.g. from about 0.2 to about 5 bar, e.g. from 0.5 to 2 bar, such as approximately 1 bar. Increasing the gas pressure will increase the concentration of hydrogen in the reaction solution. Decreasing the gas pressure will decrease the concentration of the hydrogen in the reaction solution.

For avoidance of doubt, the molecular hydrogen may also be provided in the form of a solution (e.g. an aqueous solution, e.g. comprising buffer salts as described in more detail here) in which molecular hydrogen is dissolved.

When the reductant is molecular hydrogen, the hydrogen may be provided as a mixture of hydrogen and other gases such as CO, CO₂, air, O₂, N₂, Ar, etc. When provided as a mixture, the mixture may comprise from about 0.1% to about 99% hydrogen, such as from 1% to about 95%, e.g. from about 2% to about 80% H₂, such as from about 5% to about 50% H₂. An exemplary mixture for example may comprise 50% hydrogen and 50% N₂.

When the reductant is molecular hydrogen, the hydrogen may be of any suitable purity. For example, hydrogen of 99% purity or greater (e.g. 99.9%, 99.99% or 99.999%) may be used when it is important to control impurity levels in the final product mixture. In other aspects, lower purity hydrogen may be used when it is not so important to control impurity levels in the final product mixture. For example, relatively low purity hydrogen may be provided in the form of “syngas”. Syngas produced by coal gasification generally is a mixture of 30 to 60% carbon monoxide, 25 to 30% hydrogen, 5 to 15% carbon dioxide, and 0 to 5% methane, and may optionally comprise lesser amount of other gases also. Hydrogen waste stream gas from other industrial processes may also typically be used. Accordingly, the methods of the present disclosure are ideally suited to being exploited as part of a larger reaction sequence involving a reaction step comprising hydrogen either as a reactant or as a product, with waste hydrogen from such a reaction step being used as the molecular reductant in the disclosed methods.

When the reductant is molecular hydrogen or an isotope thereof, the molecular hydrogen or isotope thereof may be provided from any suitable source, such as a gas cylinder. Alternatively, molecular hydrogen or an isotope thereof can be produced in situ e.g. by electrolysis of water.

When the molecular reductant is carbon monoxide, suitable isotopes thereof include ¹²C¹⁶O, ¹³C¹⁶O, ¹⁴C¹⁶O, ¹²C¹⁷O, ¹³C¹⁷O, ⁴C¹⁷O, ¹²C¹⁸O, ¹³C¹⁸O and ⁴C¹⁸O. Preferably, the carbon monoxide is ¹²C¹⁶O.

Typically, when the reductant is carbon monoxide, the carbon monoxide is provided in the form of a gas. The gas may be mixed with an aqueous solution in which the biocatalyst and target compound as well as optionally other reaction components are present. At 1 bar CO the solubility of CO in water is 0.95 mM. In other words, when the reductant is carbon monoxide provided in the form of molecular carbon monoxide gas, the concentration of carbon monoxide in solution (i.e. that under which the oxidoreductase operates) is 0.95 mM carbon monoxide. Other pressures may also be used. For example, the gas pressure in the reaction vessel may be from 0.01 to about 100 bar, such as from 0.1 to 10 bar, e.g. from about 0.2 to about 5 bar, e.g. from 0.5 to 2 bar, such as approximately 1 bar. Increasing the gas pressure will increase the concentration of carbon monoxide in the reaction solution. Decreasing the gas pressure will decrease the concentration of the carbon monoxide in the reaction solution.

For avoidance of doubt, the carbon monoxide may also be provided in the form of a solution (e.g. an aqueous solution, e.g. comprising buffer salts as described in more detail here) in which carbon monoxide is dissolved.

When the reductant is carbon monoxide, the carbon monoxide may be provided as a mixture of carbon monoxide and other gases such as H₂, CO₂, air, O₂, N₂, Ar, etc. When provided as a mixture, the mixture may comprise from about 0.1% to about 99% CO, such as from 1% to about 95%, e.g. from about 2% to about 80% H₂, such as from about 5% to about 50% CO. An exemplary mixture for example may comprise 50% CO and 50% N₂.

When the reductant is CO, the CO may be of any suitable purity. For example, CO of 99% purity or greater (e.g. 99.9%, 99.99% or 99.999%) may be used when it is important to control impurity levels in the final product mixture. In other aspects, lower purity CO may be used when it is not so important to control impurity levels in the final product mixture. For example, relatively low purity CO may be provided in the form of “syngas”, as described above. CO waste stream gas from other industrial processes may also typically be used. Accordingly, the methods of the present disclosure are ideally suited to being exploited as part of a larger reaction sequence involving a reaction step comprising CO either as a reactant or as a product, with waste CO from such a reaction step being used as the molecular reductant in the disclosed methods.

When the reductant is CO or an isotope thereof, the CO or isotope thereof may be provided from any suitable source, such as a gas cylinder.

When the molecular reductant is formate, suitable isotopes thereof include carbon isotopes such as ¹²C-formate, ¹³C-formate, and ¹⁴C-formate; oxygen isotopes such as ¹⁶O-formate, ¹⁷O-formate, and ¹⁸O-formate; and hydrogen isotopes such as ¹H-formate, ²H-formate and ³H-formate. Preferably, the formate consists of ¹²C, ¹⁶O and ¹H.

Typically, when the reductant is formate, the formate is provided as a metal formate salt or as formic acid. Examples of formate salts include lithium formate, sodium formate, potassium formate, magnesium formate, calcium formate, ammonium formate, and the like. Formic acid may be provided as an aqueous solution comprising from about 1% to about 99% formic acid, or may be provided in substantially pure form (e.g. at least 90%, at least 95%, at least 97%, at least 98%, at least 99% or at least 99.9% pure).

When the reductant is formate (or formic acid), the formate (or formic acid) may be present in the reaction solution in a concentration of from about 1 μM to about 1 M, e.g. from about 10 μM to about 100 mM, e.g. from about 100 μM to about 10 mM e.g. about 1 mM. Other components such as buffer salts etc may also be present.

Oxidoreductase enzyme

As discussed in more detail herein, in the disclosed methods the target compound is contacted with an oxidoreductase enzyme or a functional fragment or derivative thereof supported on a support material. The support material is typically electronically conductive or semi-conductive. Suitable support materials are described in more detail herein.

The oxidoreductase enzyme is typically in electronic contact with the support material. Typically, the active site of the oxidoreductase enzyme is in electronic communication with the support material. In other words, the biocatalyst is configured such that electrons can be transferred from the active site of the oxidoreductase enzyme (i.e. the active site at which the oxidation of the molecular reductant is catalysed) to the support material.

Typically, electrons may be transferred from the oxidoreductase enzyme (e.g. from the active site of the oxidoreductase enzyme) to the support material via an intramolecular electronically-conducting pathway. In other words, the electron transfer from the molecular reductant to the support material via the oxidoreductase enzyme is typically a direct electron transfer. While the invention does embrace the use of electron mediators (e.g. redox active dyes such as methyl or benzyl viologen) to mediate electron transfer from the molecular reductant to the support material, the electron transfer is typically not mediated by electron transfer agents such as mediators, e.g. is typically not mediated by a redox active dye such as methyl or benzyl viologen.

Typically, the electron transfer from the molecular reductant to the support material comprises direct electron transfer through the oxidoreductase enzyme. Typically, the direct electron transfer is via an intramolecular electronically-conducting pathway. Typically, the intramolecular electronically-conducting pathway comprises a series of [FeS] clusters. As those skilled in the art will appreciate, [FeS]-clusters include [3Fe4S] and [4Fe4S] clusters. [FeS] clusters may be described as distal, proximal or medial clusters. The notation “distal” and “proximal” in this context is routine in the art. For a protein which contains an active site and a chain or series of [FeS] clusters, the proximal cluster is the [FeS] cluster at closest proximity to the active site. The distal cluster is the [FeS] cluster closest to a solvent-accessible surface of the protein, and thus furthest away from the active site. [FeS] clusters between the proximal and distal clusters are referred to as medial clusters. The distal cluster is often solvent accessible. Thus, in some embodiments, the electron transfer is via a chain of [FeS] clusters from the active site to the proximal cluster, from the proximal cluster to the distal cluster (optionally via one or more medial clusters), and from the distal cluster to the support material. Without being bound by theory, each step in the electron transfer may be an electron-tunnelling step.

In some embodiments the oxidoreductase enzyme comprises a native electron transfer partner such as a cytochrome (such as the cytochrome of SEQ ID NO: 22 (or a functional fragment, derivative or variant thereof). The cytochrome may be comprised in the oxidoreductase enzyme in a position for electron transfer to the distal [FeS] cluster. In other words the cytochrome may be positioned at the distal end of the series of [FeS] clusters.

Typically, the oxidoreductase enzyme is a hydrogenase enzyme, a carbon monoxide dehydrogenase enzyme and/or a formate dehydrogenase enzyme, or a functional fragment or derivative thereof.

The oxidoreductase enzyme is preferably selected or modified to catalyze the oxidation of the molecular reductant close to the thermodynamic reduction potential Eº of the reductant. For example, when the reductant is molecular hydrogen, the oxidoreductase enzyme is preferably a hydrogenase and is preferably selected or modified to catalyze the oxidation of the hydrogen close to the thermodynamic reduction potential Eº of the 2H⁺/H₂ couple (“Eº (2H⁺/H₂)”) under the experimental conditions. (Those skilled in the art will appreciate that Eº (2H⁺/H₂)=−0.413 V at 25° C., pH 7.0 and 1 bar H₂, and varies according to the Nernst equation). Preferably, the oxidoreductase enzyme is selected or modified to catalyze H₂ or ^{x}H₂ oxidation at applied potentials of less than 100 mV more positive than Eº (2H⁺/H₂); more preferably at applied potentials of less than 50 mV more positive than Eº (2H⁺/H₂). Methods of determining the ability of an enzyme to catalyze H₂ oxidation close to E (2H⁺/H₂) under the experimental conditions at issue are routine for those skilled in the art and are, for example, described in Vincent et al, J. Am Chem Soc. (2005) 127, 18179-18189.

When the reductant is CO, the oxidoreductase enzyme is preferably a carbon monoxide dehydrogenase (CODH) and is preferably selected or modified to catalyze the oxidation of the CO close to the thermodynamic reduction potential Eº of the CO₂/CO couple (“Eº (CO₂/CO)”) under the experimental conditions. (Those skilled in the art will appreciate that Eº (CO₂/CO)=−0.53 V at 25° C.). Preferably, the oxidoreductase enzyme is selected or modified to catalyze CO oxidation at applied potentials of less than 100 mV more positive than Eº (CO₂/CO); more preferably at applied potentials of less than 50 mV more positive than Eº (CO₂/CO). Similarly, when the reductant is formate, the oxidoreductase enzyme is preferably a formate dehydrogenase and is preferably selected or modified to catalyze the oxidation of the formate close to the thermodynamic reduction potential Eº of the CO₂/formate couple (“Eº (CO₂/formate)”) under the experimental conditions. (nose skilled in the art will appreciate that Eº (CO₂/formate)=−0.42V at 25° C.). Preferably, the oxidoreductase enzyme is selected or modified to catalyze formate oxidation at applied potentials of less than 100 mV more positive than Eº (CO₂/formate); more preferably at applied potentials of less than 50 mV more positive than Eº (CO₂/formate).

In some embodiments the oxidoreductase enzyme is selected or modified to oxidise the reductant at a potential more negative than the reduction potential of the target compound.

The oxidoreductase enzyme is typically selected or modified to be highly active. For example, the oxidoreductase enzyme may be selected or modified to have efficient substrate turnover.

Enzyme turnover can be calculated in a number of ways. The Total Turnover Number (TTN, also known as the TON) is a measure of the number of moles of product per mole of enzyme. As those skilled in the art will appreciate, the TTN thus indicates the number of times that the enzyme has turned over (i.e. has oxidised a molecule of the molecular reductant). Preferably, the TTN of the oxidoreductase enzyme is at least 10, such as at least 100, more preferably at least 1000 e.g. at least 10,000 or at least 100,000, such as at least 1,000,000, preferably at least 10⁷ such as at least 10⁸, e.g. at least 10⁹.

The Turnover Frequency (TOF) is a measure of the number of moles of product generated per second per mole of enzyme present. Accordingly, in the methods provided herein, the TOF indicates the number of moles of oxidised reductant generated per second per mole of the oxidoreductase enzyme. Accordingly, the TOF is identified with the number of catalytic cycles undertaken by each enzyme molecule per second. Preferably, in the methods of the invention, the first polypeptide has a TOF of 0.1 to 1000 s⁻¹, more preferably 1 to 100 s⁻¹ such as from about 10 to about 50 s⁻¹.

When the supported biocatalyst comprises a hydrogenase enzyme, any suitable hydrogenase can be used. The hydrogenase may comprise an active site comprising iron atoms (as in the [FeFe]— hydrogenases) or both nickel and iron atoms (as in the [NiFe]— and [NiFeSe]— hydrogenases). Preferably, the hydrogenase comprises an active site comprising both nickel and iron atoms. Suitable proteins are described below.

When the supported biocatalyst comprises a hydrogenase enzyme, the hydrogenase enzyme is typically an uptake hydrogenase or a hydrogen-sensing hydrogenase. Uptake hydrogenases are used by organisms in vivo to generate energy by oxidation of molecular hydrogen in their environment. In vivo, they link oxidation of H₂ to reduction of anaerobic acceptors such as nitrate and sulfate, or O₂. Typically, uptake hydrogenases comprise a signal peptide (often of length from about 30 to about 60 amino acid residues) at the N terminus of the small subunit. Typically, the signal peptide comprises a [DENST]RRxFxK motif. Hydrogen sensing hydrogenases (also known as regulatory hydrogenases) are used by organisms in vivo to sense hydrogen levels in order to control biosynthesis of uptake hydrogenases in response to H₂. Regulatory hydrogenases typically do not comprise the signal peptide characteristic of uptake hydrogenases. Regulatory hydrogenases are often insensitive to O₂.

Typically, a hydrogenase for use in the disclosed methods is selected or modified to be oxygen tolerant. Oxygen tolerant hydrogenases are capable of oxidising H₂ or ^{x}H₂ in the presence of oxygen, such as in the presence of at least 0.01% O₂, preferably at least 0.1% O₂, more preferably at least 1% O₂, such as at least 5% O₂, e.g. at least 10% O₂ such as at least 20% O₂ or more whilst retaining at least 1%, preferably at least 5%, such as at least 10%, preferably at least 20%, more preferably at least 50% such as at least 80% e.g. at least 90% preferably at least 95% e.g. at least 99% of their H₂-oxidation activity under anaerobic conditions. Various oxygen-tolerant hydrogenases are known to those skilled in the art.

Typically, a hydrogenase for use in the disclosed methods does not comprise a native flavin active site for NAD(P)⁺ reduction (also known as a prosthetic group), although such hydrogenases are not excluded. Some known hydrogenases do comprise such an active site, such as the soluble hydrogenase (SH) enzymes from R. eutropha, Rhodococcus opacus, Hydrogenophilus thermoluteolus and Pyrococcus furiosus. However, without being bound by theory, it is believed that hydrogenases lacking such prosthetic groups typically have increased stability compared to hydrogenases comprising such prosthetic groups. Examples of hydrogenases lacking a flavin prosthetic group include Escherichia coli hydrogenase 1 (SEQ ID NOs:1-2), Escherichia coli hydrogenase 2 (SEQ ID NOs:3-4), Ralstonia eutropha membrane-bound hydrogenase (SEQ ID NOs: 5-7), Ralstonia eutropha regulatory hydrogenase (SEQ ID NOs:8-9), Aquifex aeolicus hydrogenase 1 (SEQ ID NOs:10-11), and Hydrogenovibrio marinus membrane-bound hydrogenase (SEQ ID NOs: 12-13).

When the supported biocatalyst comprises a hydrogenase, the hydrogenase is often a hydrogenase of class 1 or 2b. References to hydrogenase classes such as class 1 and class 2b refer to the established Vignais classification scheme described by Vignais and Billoud, Chem. Rev. 2007, 107, 4206-4272, which is known to those skilled in the art. The hydrogenase may be any of the hydrogenases of class 1 or class 2b listed in Vignais and Billoud, Chem. Rev. 2007, 107, 4206-4272, the contents of which are incorporated by reference.

Preferably, when the supported biocatalyst comprises a hydrogenase, the hydrogenase is selected from or comprises:
i) the amino acid sequence of Escherichia coli hydrogenase 1 (SEQ ID NOs:1 and/or 2) or an amino acid sequence having at least 60% homology therewith; ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromaium vinosum membrane bound hydrogenase (SEQ ID NOs: 18 and/or 19) or an amino acid sequence having at least 60% homology therewith;
x) the amino acid sequence of Salmonella enterica serovar Typhimurium LT2 nickel-iron hydrogenase 5 (SEQ ID NO: 20 and/or 21) or an amino acid sequence having at least 60% homology therewith;
xi) the amino acid sequence of Desulfovibrio vulgaris Miyazaki F hydrogenase (SEQ ID NO: 23 and/or 24) or an amino acid sequence having at least 60% homology therewith;
xii) the amino acid sequence of Clostridium beijerinckii SM10 (CbA5H) [FeFe]-hydrogenase (KX147468) (SEQ ID NO: 25); Clostridium beijerinckii ATCC 51743 [FeFe]-Hydrogenase (Cbei_4110) (SEQ ID NO: 26); Clostridium beijerinckii [FeFe]-Hydrogenase (Cbei_1773) (SEQ ID NO: 27); or Clostridium beijerinckii [FeFe]-Hydrogenase (Cbei_3796) (SEQ ID NO: 28) or an amino acid sequence having at least 60% homology therewith;
xiii) the amino acid sequence of Clostridium pasteurianum [FeFe]-Hydrogenase (hydA) (SEQ ID NO: 29) or an amino acid sequence having at least 60% homology therewith;
xiv) the amino acid sequence of Chlamydomonas reinhardtii [FeFe]-hydrogenase (hyd1) (SEQ ID NO: 30) or an amino acid sequence having at least 60% homology therewith;
xv) the amino acid sequence of Chorella variabilis [FeFe]-hydrogenase (SEQ ID NO: 31 and/or 32) or an amino acid sequence having at least 60% homology therewith;
xvi) the amino acid sequence of Ralstonia eutropha soluble hydrogenase moiety (SEQ ID NOs: 33 and/or 34) or an amino acid sequence having at least 60% homology therewith;
xvii) the amino acid sequence of Rhodococcus opacus soluble hydrogenase moiety (SEQ ID NOs: 35 and/or 36) or an amino acid sequence having at least 60% homology therewith;
xviii) the amino acid sequence of Desulfovibrio fructosovorans membrane bound hydrogenase (SEQ ID NOs: 37 and/or 38) or an amino acid sequence having at least 60% homology therewith;
xix) the amino acid sequence of Clostridium acetobutylicum iron-iron hydrogenase (SEQ ID NOs: 39) or an amino acid sequence having at least 60% homology therewith;
xx) the amino acid sequence of Desulfomicrobium baculatum nickel-iron selenium hydrogenase (SEQ ID NOs: 40 and/or 41) or an amino acid sequence having at least 60% homology therewith;
xxi) the amino acid sequence of Hydrogenophilus thermoluteolus soluble hydrogenase moiety (SEQ ID NOs: 42 and/or 43) or an amino acid sequence having at least 60% homology therewith;
xxii) the amino acid sequence of Desulfovibrio gigas Periplasmic [NiFe]hydrogenase (SEQ ID NOs: 44 and/or 45) or an amino acid sequence having at least 60% homology therewith; or
xxiii) the amino acid sequence of Pyrococcus furiosus soluble alpha subunit (SEQ ID NOs: 46) or an amino acid sequence having at least 60% homology therewith; or a functional fragment, derivative or variant thereof.

More preferably, when the supported biocatalyst comprises a hydrogenase, the hydrogenase is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromatium vinosum membrane bound hydrogenase (SEQ ID NOs: 18 and/or 19) or an amino acid sequence having at least 60% homology therewith;
x) the amino acid sequence of Salmonella enterica serovar Typhimurium LT2 nickel-iron hydrogenase 5 (SEQ ID NO: 20 and/or 21) or an amino acid sequence having at least 60% homology therewith;
xi) the amino acid sequence of Desulfovibrio vulgaris Miyazaki F hydrogenase (SEQ ID NO: 23 and/or 24) or an amino acid sequence having at least 60% homology therewith;
xii) the amino acid sequence of Clostridium beijerinckii SM10 (CbA5H) [FeFe]-hydrogenase (KX147468) (SEQ ID NO: 25); Clostridium beijerinckii ATCC 51743 [FeFe]-Hydrogenase (Cbei_4110) (SEQ ID NO: 26); Clostridium beijerinckii [FeFe]-Hydrogenase (Cbei_1773) (SEQ ID NO: 27); or Clostridium beijerinckii [FeFe]-Hydrogenase (Cbei_3796) (SEQ ID NO: 28) or an amino acid sequence having at least 60% homology therewith; or
xiii) the amino acid sequence of Clostridium acetobutylicum iron-iron hydrogenase (SEQ ID NOs: 39) or an amino acid sequence having at least 60% homology therewith.

Still more preferably, when the supported biocatalyst comprises a hydrogenase, the hydrogenase is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Aquifex aeolicus hydrogenase 1 (SEQ ID NO:10 and/or 11) or an amino acid sequence having at least 60% homology therewith;
v) the amino acid sequence of Hydrogenovibrio marinus hydrogenase (SEQ ID NOs: 12 and/or 13) or an amino acid sequence having at least 60% homology therewith;
vi) the amino acid sequence of Alteromonas macleodii hydrogenase (SEQ ID NOs: 16 and/or 17) or an amino acid sequence having at least 60% homology therewith; or
vii) the amino acid sequence of Salmonella enterica serovar Typhimurium LT2 nickel-iron hydrogenase 5 (SEQ ID NO: 20 and/or 21) or an amino acid sequence having at least 60% homology therewith;
   or a functional fragment, derivative or variant thereof.

Most preferably, when the supported biocatalyst comprises a hydrogenase, the hydrogenase comprises the amino acid sequence of Escherichia coli hydrogenase 1 (SEQ ID NOs:1 and/or 2) or an amino acid sequence having at least 60% homology therewith; or a functional fragment, derivative or variant thereof.

Also most preferably, in another embodiment, when the supported biocatalyst comprises a hydrogenase, the hydrogenase comprises the amino acid sequence of Clostridium beijerinckii SM10 (CbA5H) [FeFe]-hydrogenase (KX147468) (SEQ ID NO: 25); Clostridium beijerinckii ATCC 51743 [FeFe]-Hydrogenase (Cbei_4110) (SEQ ID NO: 26); Clostridium beijerinckii [FeFe]-Hydrogenase (Cbei_1773) (SEQ ID NO: 27); or Clostridium beijerinckii [FeFe]-Hydrogenase (Cbei_3796) (SEQ ID NO: 28) or an amino acid sequence having at least 60% homology therewith.

Preferably, when the supported biocatalyst comprises a carbon monoxide dehydrogenase, the carbon monoxide dehydrogenase is selected from or comprises:
i) the amino acid sequence of Desulfovibrio vulgaris Hildenborough Carbon monoxide dehydrogenase (cooS) (SEQ ID NO: 47) or an amino acid sequence having at least 60% homology therewith;
ii) the amino acid sequence of Desulfovibrio vulgaris Miyazaki Carbon monoxide dehydrogenase (DvMF) (SEQ ID NO: 48) or an amino acid sequence having at least 60% homology therewith;
iii) the amino acid sequence of Desulfovibrio psychrotolerans Carbon monoxide dehydrogenase (cooS) (SEQ ID NO: 49) or an amino acid sequence having at least 60% homology therewith;
iv) the amino acid sequence of Desulfoluna spongiiphila Carbon monoxide dehydrogenase (SAMN05216233) (SEQ ID NO: 50) or an amino acid sequence having at least 60% homology therewith;
v) the amino acid sequence of Halodesulfovibrio spirochaetisodalis Carbon monoxide dehydrogenase (SP90) (SEQ ID NO: 51) or an amino acid sequence having at least 60% homology therewith;
vi) the amino acid sequence of Desulfovibrio desulfuricans Carbon monoxide dehydrogenase (Ddes) (SEQ ID NO: 52) or an amino acid sequence having at least 60% homology therewith;
vii) the amino acid sequence of Desulfurivibrio alkaliphilus Carbon monoxide dehydrogenase (DaAHT2) (SEQ ID NO: 53) or an amino acid sequence having at least 60% homology therewith;
viii) the amino acid sequence of Pseudodesulfovibrio aespoeensis Carbon monoxide dehydrogenase (Daes) (SEQ ID NO: 54) or an amino acid sequence having at least 60% homology therewith;
ix) the amino acid sequence of Desulfovibrio alaskensis Carbon monoxide dehydrogenase (Dde_3028) (SEQ ID NO: 55) or an amino acid sequence having at least 60% homology therewith;
x) the amino acid sequence of Desulfovibrio ferrophilus Carbon monoxide dehydrogenase (DFE_2686) (SEQ ID NO: 56) or an amino acid sequence having at least 60% homology therewith;
xi) the amino acid sequence of Carboxydothermus hydrogenoformans Carbon monoxide dehydrogenase 2 (cooS2) (SEQ ID NO: 57) or an amino acid sequence having at least 60% homology therewith;
xii) the amino acid sequence of Desulfofundulus salinum Carbon monoxide dehydrogenase (cooS) (SEQ ID NO: 58) or an amino acid sequence having at least 60% homology therewith;
xiii) the amino acid sequence of Caldanaerobacter subterraneus Carbon monoxide dehydrogenase tengcongensis (TTE1708) (SEQ ID NO: 59) or an amino acid sequence having at least 60% homology therewith;
xiv) the amino acid sequence of Thermanaeromonas toyohensis Carbon monoxide dehydrogenase (SAMN00808754_0706) (SEQ ID NO: 60) or an amino acid sequence having at least 60% homology therewith;
xv) the amino acid sequence of Desulfobulbus sp. Carbon monoxide dehydrogenase (JTO6_17280) (SEQ ID NO: 61) or an amino acid sequence having at least 60% homology therewith;
xvi) the amino acid sequence of Desulfotomaculum copahuensis Carbon monoxide dehydrogenase (A6M21_00615) (SEQ ID NO: 62) or an amino acid sequence having at least 60% homology therewith;
xvii) the amino acid sequence of Pelotomaculum propionicicum Carbon monoxide dehydrogenase (cooS2) (SEQ ID NO: 63) or an amino acid sequence having at least 60% homology therewith;
xviii) the amino acid sequence of Methylomusa anaerophila Carbon monoxide dehydrogenase (cooS2) (SEQ ID NO: 64) or an amino acid sequence having at least 60% homology therewith;
xix) the amino acid sequence of Sporomusa silvacetica Carbon monoxide dehydrogenase (cooS2) (SEQ ID NO: 65) or an amino acid sequence having at least 60% homology therewith;
xx) the amino acid sequence of Heliobacillus mobilis Carbon monoxide dehydrogenase (cooS) (SEQ ID NO: 66) or an amino acid sequence having at least 60% homology therewith;
xxi) the amino acid sequence of Desulfocucumis palustris Carbon monoxide dehydrogenase (DCCM_2691) (SEQ ID NO: 67) or an amino acid sequence having at least 60% homology therewith;
   or a functional fragment, derivative or variant thereof.

Preferably, when the supported biocatalyst comprises a formate dehydrogenase, the formate dehydrogenase is selected from or comprises:
i) the amino acid sequence of Escherichia coli Formate dehydrogenase, nitrate-inducible, major subunit (fdnG) (SEQ ID NO: 68) or an amino acid sequence having at least 60% homology therewith;
ii) the amino acid sequence of Shigella flexneri Formate dehydrogenase-N, nitrate-inducible, alpha subunit (fdnG) (SEQ ID NO: 69) or an amino acid sequence having at least 60% homology therewith;
iii) the amino acid sequence of Enterobacteriaceae bacterium Formate dehydrogenase-N subunit alpha (fdnG) (SEQ ID NO: 70) or an amino acid sequence having at least 60% homology therewith;
iv) the amino acid sequence of Salmonella typhimurium Molybdopterin oxidoreductase (fdnG) (SEQ ID NO: 71) or an amino acid sequence having at least 60% homology therewith;
v) the amino acid sequence of Citrobacter rodentium Formate dehydrogenase, nitrate-inducible, major subunit (fdnG) (SEQ ID NO: 72) or an amino acid sequence having at least 60% homology therewith;
vi) the amino acid sequence of Escherichia alba Formate dehydrogenase-N subunit alpha (fdnG) (SEQ ID NO: 73) or an amino acid sequence having at least 60% homology therewith;
vii) the amino acid sequence of Enterobacteriaceae bacterium Formate dehydrogenase-N subunit alpha (fdnG) (SEQ ID NO: 74) or an amino acid sequence having at least 60% homology therewith;
viii) the amino acid sequence of Enterobacteriaceae bacterium 4M9 Formate dehydrogenase-N subunit alpha (fdnG) (SEQ ID NO: 75) or an amino acid sequence having at least 60% homology therewith;
ix) the amino acid sequence of Erwinia sp. Formate dehydrogenase-N subunit alpha (fdnG) (SEQ ID NO: 76) or an amino acid sequence having at least 60% homology therewith;
x) the amino acid sequence of Jejubacter calystegiae Formate dehydrogenase-N subunit alpha (fdnG) (SEQ ID NO: 77) or an amino acid sequence having at least 60% homology therewith;
xi) the amino acid sequence of Moellerella wisconsensis Selenocysteine-containing formate dehydrogenase N alpha subunit (M992_0960) (SEQ ID NO: 78) or an amino acid sequence having at least 60% homology therewith;
   or a functional fragment, derivative or variant thereof.

Preferably, when the supported biocatalyst comprises an oxidoreductase enzyme which comprises or consists of one or more amino acid sequences having at least 60% homology with a specified sequence, each amino acid sequence independently has at least 70%, such as at least 80%, more preferably at least 90%, e.g. at least 95%, preferably at least 97%, such as at least 98%, preferably at least 99% homology with the specified sequence. More preferably, each amino acid sequence independently has at least 70%, such as at least 80%, more preferably at least 90%, e.g. at least 95%, preferably at least 97%, such as at least 98%, preferably at least 99% identity with the specified sequence. For avoidance of doubt, if the oxidoreductase enzyme two or more amino acid sequences, the percentage homology of each of the two or more sequences with respect to their respective specified sequences can be the same or different, preferably the same. Percentage homology and/or percentage identity are each preferably determined across the entire length of the specified reference sequence as described herein.

It will be apparent to the skilled person that an oxidoreductase enzyme may either be a single polypeptide or may comprise multiple polypeptides. An oxidoreductase enzyme may be a portion such as one or more domains of a multidomain polypeptide. For example, those skilled in the art will appreciate that oxidoreductase enzymes such as hydrogenases and carbon monoxide dehydrogenases typically comprise two or more subunits. As used herein, the term “oxidoreductase enzyme” relates to one or more of the subunits of the relevant protein. For example, when the oxidoreductase enzyme is Escherichia coli hydrogenase 1 (SEQ ID NOs:1 and/or 2), the oxidoreductase enzyme may comprise (i) SEQ ID NO: 1 but not SEQ ID NO: 2; (ii) SEQ ID NO: 2 but not SEQ ID NO: 1; or (iii) both SEQ ID NO: 1 and SEQ ID NO: 2. When the oxidoreductase enzyme is Escherichia coli hydrogenase 2 (SEQ ID NOs:3 and/or 4), the oxidoreductase enzyme may comprise (i) SEQ ID NO: 3 but not SEQ ID NO: 4; (ii) SEQ ID NO: 4 but not SEQ ID NO: 3; or (iii) both SEQ ID NO: 3 and SEQ ID NO: 4. Typically, when the oxidoreductase enzyme is a hydrogenase enzyme having two or more subunits, the oxidoreductase enzyme comprises said two or more subunits.

The oxidoreductase enzyme may be used in the form of a monomer or a multimer. For example, when the oxidoreductase enzyme comprises a hydrogenase which can exist in a monomeric or dimeric form, the oxidoreductase enzyme can be provided in the form of the monomer or the dimer. For example, Escherichia coli hydrogenase 1 may be purified either as a dimer or a monomer or a mixture thereof. When the oxidoreductase enzyme comprises Escherichia coli hydrogenase 1 (i.e. SEQ ID NOs: 1 and/or 2) the first polypeptide may be provided as a monomer (1× SEQ ID NO: 1 and/or 1× SEQ ID NO 2) or as a dimer (2× SEQ ID NO: 1 and/or 2× SEQ ID NO: 2), or as a mixture thereof. When the oxidoreductase enzyme is provided as a mixture of a monomer and dimer, the mixture typically contains from about 1% to about 99% of the monomer and from about 99% to about 1% of the dimer. Sometimes, the amount of monomer and dimer may be approximately similar, and the first polypeptide may thus comprise from about 30% to about 70% monomer and from about 70% to about 30% dimer, such as from about 40% to about 60% monomer and from about 60% to about 40% dimer. Sometimes, the oxidoreductase enzyme comprises from about 1 to about 10% monomer/about 90% to about 99% dimer, e.g. from about 1% to about 5% monomer/about 95% to about 99% dimer. Sometimes, the oxidoreductase enzyme comprises from about 1 to about 10% dimer/about 90% to about 99% monomer, e.g. from about 1% to about 5% dimer/about 95% to about 99% monomer.

It will also be apparent to those skilled in the art that the supported biocatalyst may comprise associated proteins which may for example be co-purified with the oxidoreductase enzyme. For example, when the supported biocatalyst comprises a hydrogenase of amino acid sequence of SEQ ID NO: 1 and/or 2 (or a functional fragment, derivative or variant thereof), the supported biocatalyst may further comprise a native cytochrome electron transfer partner such as the cytochrome of SEQ ID NO: 22 (or a functional fragment, derivative or variant thereof). Thus, in embodiments in which the oxidoreductase enzyme comprises SEQ ID NO: 1 and/or 2 (or a functional fragment, derivative or variant thereof), the supported biocatalyst may also comprise SEQ ID NO: 22 (or a functional fragment, derivative or variant thereof).

### Polypeptides Used in the Invention

Methods for expression of proteins in cellular (e.g. microbial) expression systems are well known and routine to those skilled in the art. For example, the oxidoreductase enzyme can be independently isolated from its host organisms using routine purification methods. For example, host cells can be grown in a suitable medium. Lysing of cells allows internal components of the cells to be accessed. Membrane proteins can be solubilised with detergents such as Triton X (e.g. Triton X-114, (1,1,3,3-Tetramethylbutyl)phenyl-polyethylene glycol, available from Sigma Aldrich). Soluble or solubilized proteins can be isolated and purified using standard chromatographic techniques such as size exclusion chromatography, ion exchange chromatography and hydrophobic interaction chromatography. Alternatively, the oxidoreductase enzyme can be encoded in one or more nucleotide vector and subsequently expressed in an appropriate host cell (e.g. a microbial cell, such as E. coli). Purification tags such as a HIS (hexa-histidine) tag can be encoded (typically at the C- or N-terminal of the relevant polypeptide) and can be used to isolate the tagged protein using affinity chromatography for example using nickel- or cobalt-NTA chromatography. If desired, protease recognition sequences can be incorporated between the oxidoreductase enzyme and the affinity purification tag to allow the tag to be removed post expression. Such techniques are routine to those skilled in the art and are described in, for example, Sambrook et al, “Molecular Cloning: A Laboratory Manual”, Cold Spring Harbor Laboratory Press.

As described herein the oxidoreductase enzyme may be a functional fragment, derivative or variant of an enzyme or amino acid sequence. As those skilled in the art will appreciate, fragments of amino acid sequences include deletion variants of such sequences wherein one or more, such as at least 1, 2, 5, 10, 20, 50 or 100 amino acids are deleted. Deletion may occur at the C-terminus or N-terminus of the native sequence or within the native sequence. Typically, deletion of one or more amino acids does not influence the residues immediately surrounding the active site of an enzyme. Derivatives of amino acid sequences include post-translationally modified sequences including sequences which are modified in vivo or ex vivo. Many different protein modifications are known to those skilled in the art and include modifications to introduce new functionalities to amino acid residues, modifications to protect reactive amino acid residues or modifications to couple amino acid residues to chemical moieties such as reactive functional groups on linkers or substrates (surfaces) for attachment to such amino acid residues.

Derivatives of amino acid sequences include addition variants of such sequences wherein one or more, such as at least 1, 2, 5, 10, 20, 50 or 100 amino acids are added or introduced into the native sequence. Addition may occur at the C-terminus or N-terminus of the native sequence or within the native sequence. Typically, addition of one or more amino acids does not influence the residues immediately surrounding the active site of an enzyme.

Variants of amino acid sequences include sequences wherein one or more amino acid such as at least 1, 2, 5, 10, 20, 50 or 100 amino acid residues in the native sequence are exchanged for one or more non-native residues. Such variants can thus comprise point mutations or can be more profound e.g. native chemical ligation can be used to splice non-native amino acid sequences into partial native sequences to produce variants of native enzymes. Variants of amino acid sequences include sequences carrying naturally occurring amino acids and/or unnatural amino acids. Variants, derivatives and functional fragments of the aforementioned amino acid sequences retain at least some of the activity/functionality of the native/wild-type sequence. Preferably, variants, derivatives and functional fragments of the aforementioned sequences have increased/improved activity/functionality when compared to the native/wild-type sequence.

Variants of an enzyme, such as the oxidoreductase enzyme described herein, may preferably be modified to have an increased catalytic activity for their respective substrates. Preferably, the catalytic activity is increased at least 2 times, such as at least 5 times, e.g. at least 10 times, such as at least 100 times, preferably at least 1000 times. Catalytic activity can be determined in any suitable method. For example, the catalytic activity can be associated with the Michaelis constant K_{M} (with increased activity being typically associated with decreased K_{M} values) or with the catalytic rate constant, k_{cat} (with increased activity being typically associated with increased k_{cat} values).

Measuring K_{M} and k_{cat} is routine to those skilled in the art. For example, the K_{M} of a polypeptide for a substrate can be determined spectrophotometrically, e.g. by measuring absorption at 578 nm under anaerobic conditions at 30° C. in 50 mM Tris-HCl buffer, pH 8.0, containing 1 mM substrate, 5 mM benzyl viologen (oxidized; F=8.9 mM⁻¹ cm⁻¹), 90 μM dithionite, and 10 to 30 pmol of enzyme. Examples of solution assays in which the absorbance of oxidised and reduced flavins are determined are described in the examples.

Standard methods in the art may be used to determine homology. For example the UWGCG Package provides the BESTFIT program which can be used to calculate homology, for example used on its default settings (Devereux et al (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10). Software for performing BLAST analyses is publicly available through the National Center for Biotechnology Information (http://www.ncbi.nlm.nih.gov/).

Similarity can be measured using pairwise identity or by applying a scoring matrix such as BLOSUM62 and converting to an equivalent identity. Since they represent functional rather than evolved changes, deliberately mutated positions would be masked when determining homology. Similarity may be determined more sensitively by the application of position-specific scoring matrices using, for example, PSIBLAST on a comprehensive database of protein sequences. A different scoring matrix could be used that reflects amino acid chemico-physical properties rather than frequency of substitution over evolutionary time scales (e.g. charge). Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A below. Where amino acids have similar polarity, this can also be determined by reference to the hydropathy scale for amino acid side chains in Table B.

TABLE A
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, |
| | | | neutral |
| Asp | polar, hydrophilic, charged | Pro | hydrophobic, neutral |
| | (−) | | |
| Glu | polar, hydrophilic, charged | Gln | polar, hydrophilic, |
| | (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, |
| | | | hydrophobic |

TABLE B
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Preferably, sequence homology can be assessed in terms of sequence identity. Any of a variety of sequence alignment methods can be used to determine percent identity, including, without limitation, global methods, local methods and hybrid methods, such as, e.g., segment approach methods. Protocols to determine percent identity are routine procedures within the scope of those skilled in the art. Global methods align sequences from the beginning to the end of the molecule and determine the best alignment by adding up scores of individual residue pairs and by imposing gap penalties. Preferred methods include CLUSTAL W (Thompson et al., Nucleic Acids Research, 22(22) 4673-4680 (1994)) and iterative refinement (Gotoh, J. Mol. Biol. 264(4) 823-838 (1996)). Local methods align sequences by identifying one or more conserved motifs shared by all of the input sequences. Preferred methods include Match-box, (Depiereux and Feytmans, CABIOS 8(5) 501-509 (1992)); Gibbs sampling, (Lawrence et al., Science 262(5131) 208-214 (1993)); and Align-M (Van Walle et al., Bioinformatics, 20(9) 1428-1435 (2004)). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

Alignment scores for determining sequence identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Percent identity is then calculated as:

$$100 \times ( T / L )$$

where
T=Total number of identical matches
L=Length of the longer sequence plus the number of gaps introduced into the longer sequence in order to align the two sequences

### Support Material

As discussed, the disclosed methods comprise the use of a supported biocatalyst comprising an oxidoreductase enzyme or a functional fragment or derivative thereof supported on a support material.

A benefit which arises from the support of the oxidoreductase enzyme is that the supported biocatalyst can be easily removed from the reaction mixture. For example, the support(s) can be removed by sedimentation, filtration, centrifugation, or the like. Many such methods are known to those skilled in the art, e.g. filtration can be achieved using a simple filter paper to remove solid components from a liquid composition; or a mixed solid/liquid composition can be allowed to settle and the liquid then decanted from the settled solids.

The oxidoreductase enzyme (or functional fragment or derivative thereof) is typically immobilized on the support material. As used herein, the term “immobilized” embraces adsorption, entrapment and/or cross-linkage between the support and the polypeptide. Adsorption embraces non-covalent interactions including electrostatic interactions, hydrophobic interactions, and the like. A charged adsorption enhancer such as polymyxin B sulphate can be used to enhance adsorption. Entrapment embraces containment of the enzyme onto the surface of the support, e.g. within a polymeric film or in a hydrogel. Cross-linkage embraces covalent attachment, either directly between the enzyme (e.g. via amide coupling, such as via EDC/NHS and/or other coupling agents routine to those skilled in the art) or using one or more covalent cross-linkers such as thiol-terminated linkers or crosslinking reagents. Immobilization means comprising or consisting of adsorption are preferred. Combination of some or all of the above mentioned immobilization means may be used. For example, the enzyme may be linked (e.g. covalently linked) to a binding group for non-covalent adsorption onto the support material. For example, the enzyme may be covalently linked to a hydrophobic group (e.g. an aromatic or heteroaromatic group) for non-covalent interaction (e.g. via hydrophobic interaction, pi-pi stacking, etc) with the support material. Suitable binding groups are known in the art and include molecules such as pyrene and derivatives thereof.

In the disclosed methods, the support material is typically electronically conductive or semi-conductive.

Preferably, the or each support independently comprises a material comprising carbon (including doped carbon materials), a metal or metal alloy, a metal oxide (including mixed metal oxides), a metal hydroxide (including layered double hydroxides), a metal chalcogenide, a semi-conducting material (including carbon nitrides, silicates, germanium compounds and gallium compounds such as silicon carbide, doped silicon and/or doped germanium) or an electronically-conductive polymer, or mixtures thereof. As those skilled in the art will appreciated, suitable support materials can include mixtures of materials described herein, such as mixtures of metal oxides or mixed metal oxides; mixtures of carbon with metal oxides (e.g. mixtures of carbon with silica, alumina, etc). As used herein, the term “mixture” embraces both atomic mixtures such as alloys, and heterogeneous mixtures such as mixtures of particles of one or more materials disclosed herein.

Any suitable support material can be used.

More preferably, the or each support material independently comprises:
i) carbon; and/or
ii) a metal or metal alloy selected from gold, silver, tungsten, iridium, platinum, palladium, copper, titanium, brass, and steel; and/or
iii) a material selected from titanium oxide, indium oxide, tin oxide and indium tin oxide.

Still more preferably, the or each support material comprises a carbon material. Still more preferably, the or each support material independently comprises a carbon material comprising graphite, carbon nanotube(s), carbon black, activated carbon, carbon nanopowder, vitreous carbon, carbon fibre(s), carbon cloth, carbon felt, carbon paper, graphene, highly oriented pyrolytic graphite, pyrolytic graphite, doped or surface-modified carbon or doped diamond.

Examples of doped carbon materials include carbon doped with boron, nitrogen, oxygen, phosphorus, silicon, sulfur, first- or second-row transition metals (e.g. scandium, titanium, vanadium, chromium, manganese, iron, cobalt, nickel, copper, zinc, yttrium, zirconium, niobium, molybdenum, technetium, ruthenium, rhodium, palladium, silver, and cadmium; particularly cobalt, nickel and copper), post-transition metals (e.g. aluminium, gallium, indium, and tin) and/or lanthanide elements (e.g. cerium, praseodymium, neodymium, promethium, samarium, europium, gadolinium, terbium, dysprosium, holmium, erbium, thulium, ytterbium, and lutetium). The dopant material may optionally be present in the material at a concentration of from about 0.001 at % to about 10 at %, e.g. from 0.01 at % to about 1 at % such as about 0.1 at %. Preferred examples of doped carbon materials include carbon doped with boron, nitrogen, cobalt, nickel and/or copper. The carbon in a doped carbon material may be selected from graphite, carbon nanotube(s), carbon black, activated carbon, carbon nanopowder, vitreous carbon, carbon fibre(s), carbon cloth, carbon felt, carbon paper, graphene, highly oriented pyrolytic graphite, pyrolytic graphite, and diamond (optionally surface-modified diamond).

For example, when the carbon material comprises doped graphene, the graphene may preferably be doped with one or more dopants selected from nitrogen, boron, sulphur, oxygen, silicon, lanthanide elements and transition-metals. When the carbon material comprises doped carbon nanotube(s), the carbon nanotube(s) may preferably be doped with one or more dopants selected from nitrogen, boron, sulphur, oxygen, silicon, lanthanide elements and transition-metals. When the carbon material comprises doped diamond, the diamond may preferably be doped with one or more dopants selected from nitrogen, boron, sulphur, oxygen and silicon. When the carbon material comprises doped carbon black, the carbon black may preferably be doped with one or more dopants selected from nitrogen, boron, sulphur, oxygen, silicon, lanthanide elements and transition-metals. When the carbon material comprises doped activated carbon, the activated carbon may preferably be doped with one or more dopants selected from nitrogen, boron, sulphur, oxygen, silicon, lanthanide elements and transition-metals.

Examples of surface-modified carbon materials include carbon materials comprising acidic surface groups (e.g. carboxylic acids, carboxylic acid anhydrides, lactones, hydroxyls); basic surface groups (e.g. amides, imides, lactames, carbonyls, amines, imines, pyrrolic and pyridinic groups), and surface groups such as nitro, diazonium, nitroso, fluoro, chloro, bromo, and iodo groups. Functional groups on the carbon material can be introduced by methods such as oxidation (e.g. using reagents such as using nitric acid, sulfuric acid, hydrogen peroxide, potassium permanganate, air, ozone, plasma, etc.), amidation, silanization, silylation, polymer grafting, polymer wrapping, surfactant adsorption, and encapsulation. Such modifications can be generally applied to carbon materials as disclosed herein. In some embodiments, acid oxidation to produce carboxylic acid groups on the carbon surface is preferred. Thus, in some embodiments preferred support materials include carbon black with carboxylic acid surface groups, activated carbon with carboxylic acid surface groups and carbon nanotube(s) with carboxylic acid surface groups.

Most preferably, the or each support material independently comprises a carbon material comprising graphite (or highly oriented pyrolytic graphite or pyrolytic graphite), activated carbon or carbon black; most preferably activated carbon or carbon black.

Preferably, the or each support is an electronically conducting particle. Preferred electronically conducting particles comprise materials described herein. Preferably, when the or each support comprise particles, the particles have a particle size of from about 1 nm to about 100 μm, such as from about 10 nm to about 10 μm e.g. from about 100 nm to about 1 μm. Methods of determining particle size are routine in the art and include, for example, dynamic light scattering. Suitable electronically conducting particles for use in the methods of the invention include conductive carbon black particles such as “Black Pearls 2000” particles available from Cabot corp (Boston, Mass., USA); and activated carbon such as Activated Charcoal “DARCO” (˜100 mesh).

### Target Compound

As discussed herein, the disclosed methods comprise reducing a reducible functional group (e.g. a nitrogen-containing functional group) in a target compound.

Exemplary nitrogen-containing functional groups include nitro groups (R—NO₂), azide groups (R—N₃), hydroxylamine groups (R—NR′OH), nitroso groups (R—NO), nitrile groups (R—CN), diazo groups (R—CR′═N₂), diazonium groups (R—N₂*), isocyanide groups (R—NC); isothiocyanate groups (R—NCS), isocyanate groups (R—N═C═O), hydrazone groups (R—CR′═N—NR′), hydrazine groups (R—NR′—NR′₂), amidine groups (R—C(NR′)NR′₂), azo groups (R—N═N═R′), and guanidine groups (R—NR′—C(NR′)—NR′₂), etc; wherein R represents the remainder of the target compound and each R independently represents a group such as H or hydrocarbyl, e.g, alkyl, e.g. C₁₋₆ or C₁₋₄ alkyl such as methyl or ethyl; typically H). More typically, the nitrogen-containing functional group is selected from nitro groups (R—NO₂⁺), azide groups (R—N₃), hydroxylamine groups (R—NR′OH), nitroso groups (R—NO), nitrile groups (R—CN), diazo groups (R—CR′═N₂), diazonium groups (R—N₂), isocyanide groups (R—NC); isothiocyanate groups (R—NCS), isocyanate groups (R—N═C═O), hydrazone groups (R—CR′═N—NR′), and hydrazine groups (R—NR′—NR′₂). Still more typically, the nitrogen-containing functional group is selected from nitro groups (R—N₀₂), azide groups (R—N₃), hydroxylamine groups (R—NR′OH), nitrile groups (R—CN), diazo groups (R—N₂⁺), isocyanide groups (R—NC). Sill more typically, the nitrogen-containing functional group is selected from nitro groups (R—NO₂) and azide groups (R—N₃). Most typically, the nitrogen-containing functional group is a nitro group.

The target compound may be any target compound, and selection of appropriate target compounds for use in accordance with the disclosed methods is an operational parameter of the disclosed methods and is well within the capacity of those skilled in the art.

The methods of the invention find utility particularly in the production of complex products such as in synthesis or derivatization of natural products, and in pharmaceutical production. Accordingly, the target compound may be a pharmaceutical intermediate, such as an oxidised form of a therapeutic drug wherein said drug comprises an amine group and wherein the oxidised form of said drug comprises an oxidised form of said amine group, such as a nitro group. The characterisation of the products obtained from the methods of the invention is well within the capacity of those skilled in the art. For example, products can be characterised by chemical analytical techniques such as IR spectroscopy, NMR, GC (including chiral-phase GC), polarimetry, mass spectroscopy, HPLC, etc. Exemplary methods are provided in the examples.

The target compound is typically an organic compound and typically has a molecular weight of from about 20 to about 2000 g/mol, such as from about 50 to about 1000 g/mol e.g. from about 100 to about 700 g/mol.

Often, the nitrogen-containing functional group is attached (e.g. covalently attached as a substituent) to an aromatic group comprised in the target compound (R), although non-aromatic groups are also embraced in the methods of the invention. Aromatic groups include hydrocarbyl aromatic groups (such as benzene) and heteroaromatic groups (such as pyridine), which may be further substituted. Exemplary non-aromatic groups include alkyl, alkenyl, carbocyclic and heterocyclic groups, which may be further substituted.

Conversion of both aromatic and aliphatic compounds is demonstrated in the examples with a range of oxidoreductase enzymes, demonstrating the broad applicability of the disclosed methods to a wide variety of target compounds.

A target compound may typically comprise a plurality of such groups bonded together, with optional further substitutions. The substituents that may be present on a target compound (e.g. on a group in a target compound as disclosed herein) are not limited and include for example H, halo (e.g. F, Br, Cl, I), —OR′ (e.g. OH or OMe), —NR′₂, SR′ (e.g. SH), SOR′, SO₂R′, C(O)R′, C(O)OR′, and C(O)NR′₂. Further substituents that may be present on a target compound (e.g. on a group in a target compound as disclosed herein) include optionally substituted alkyl groups, e.g. C₁ to C₆ (e.g. C₁ to C₄) alkyl groups which are unsubstituted or substituted with OR′ or halogen (e.g. F); CN; optionally substituted alkenyl groups, e.g. C₁ to C₆ (e.g. C₁ to C₄) alkyl groups which are unsubstituted or substituted with OR′ or halogen (e.g. F, for example CF₃). Typically a group in a target compound as disclosed herein may comprise 1, 2, 3 or more substituents in addition to the nitrogen-containing functional group.

As used herein, R may comprise or consist of any suitable aromatic or aliphatic group or combination of groups. Suitable groups are defined herein.

For avoidance of doubt, a plurality of nitrogen-containing functional groups may be comprised in the target molecule and may be reduced according to the methods disclosed herein.

An alkyl group such as a C₁ to C₂₀ alkyl group is a linear or branched alkyl group containing from 1 to 20 carbon atoms. Similarly, a C₁ to C₁₀ alkyl group is a linear or branched alkyl group containing from 1 to 10 carbon atoms. A C₁ to C₁₀ alkyl group is often a C₁ to C₆ alkyl group or a C₁ to C₄ alkyl group. Examples of C₁ to C₄ alkyl groups include methyl, ethyl, n-propyl, iso-propyl, n-butyl, iso-butyl, sec-butyl, and tert-butyl. As used herein, the term “alkyl” embraces “alkylene”, defined as a bidentate moiety derived by removing two hydrogen atoms from an alkane.

An alkenyl group such as a C₂ to C₂₀ alkenyl group is a linear or branched alkenyl group containing from 2 to 20 carbon atoms. Similarly, a C₂ to C₁₀ alkenyl group is a linear or branched alkneyl group containing from 2 to 10 carbon atoms and having one or more, e.g. one or two, typically one double bonds. A C₂ to C₁₀ alkenyl group is often a C₂ to C₆ alkenyl group or a C₂ to C₄ alkenyl group. Examples of C₂-C₄ alkenyl groups include ethenyl, propenyl and butenyl. As used herein, the term “alkenyl” embraces “alkenylene”, defined as a bidentate moiety derived by removing two hydrogen atoms from an alkene.

A carbocyclic group is a cyclic hydrocarbon typically containing from 3 to 20, e.g. from 3 to 10 carbon atoms. A carbocyclic group may be saturated or partially unsaturated, but is typically saturated; when unsaturated the group typically comprises 1 or 2, e.g. 1 double bond. Examples of saturated carbocyclic groups include cyclopropyl, cyclobutyl, cyclopentyl and cyclohexyl groups.

A heterocyclic group is a cyclic group typically containing from 3 to 20, e.g. from 3 to 10 atoms selected from C, O, N and S in the ring, including at least one heteroatom, and typically one or two heteroatoms. A heterocyclic group may be saturated or partially unsaturated, but is typically saturated; when unsaturated the group typically comprises 1 or 2, e.g. 1 double bond. Examples of heterocyclic groups include piperazine, piperidine, morpholine, 1,3-oxazinane, pyrrolidine, imidazolidine, oxazolidine; tetrahydropyrazine, tetrahydropyridine, dihydro-1,4-oxazine, tetrahydropyrimidine, dihydro-1,3-oxazine, dihydropyrrole, dihydroimidazole, dihydrooxazole, indoline, 2,3-dihydrobenzofuran, 2,3-dihydrobenzo[b]thiophene, 2,3-dihydro-1H-benzo[d]imidazole, 2,3-dihydrobenzo[d]oxazole, 2,3-dihydrobenzo[d]thiazole, benzo[d][1,3]dioxole, 4,5,6,7-tetrahydrothiazolo[5,4-c]pyridine and 4,5,6,7-tetrahydrothiazolo[4,5-c]pyridine, 1,2,3,4-tetrahydroquinoline, 1,2,3,4-tetrahydroisoquinoline, chromane, isochromane, thiochromane, isothiochromane, 1,2,3,4-tetrahydroquinoxaline, 1,2,3,4-tetrahydroquinazoline, 1,4-dihydro-2H-benzo[d][1,3]oxazine, 3,4-dihydro-2H-benzo[b][1,4]oxazine, 3,4-dihydro-2H-benzo[b][1,4]thiazine, 1,4-dihydro-2H-benzo[d][1,3]thiazine, 4H-benzo[d][1,3]dioxine and 2,3-dihydrobenzo[b][1,4]dioxine, including quaternised derivatives thereof.

A hydrocarbyl aromatic group is typically a C₆ to C₂₀ aryl group, more typically a C₆ to C₁₀ aryl group, which is a substituted or unsubstituted, monocyclic or fused polycyclic aromatic group containing from 6 to 20 (e.g. from 6 to 10) carbon atoms in the ring portion. Examples include monocyclic groups such as phenyl and fused bicyclic groups such as naphthyl and indenyl. Phenyl (benzene) is preferred.

A heteroaromatic group is typically a 5- to 10-membered heteroaryl group, which is a substituted or unsubstituted monocyclic or fused polycyclic aromatic group containing from 5 to 10 atoms in the ring portion, including at least one heteroatom, for example 1, 2 or 3 heteroatoms, typically selected from O, S and N. Examples of heteroaryl groups include pyrrole, furan, thiophene, imidazole, oxazole, thiazole, pyridine, pyridazine, pyrimidine, pyrazine, indole, benzothiophene, benzofuran, benzoxazole, benzothiazole, benzimidazole, imidazo[1,2-a]pyridine, [1,2,4]triazolo[1,5-a]pyridine, imidazo[1,2-a]pyrazine, quinoline, isoquinoline, quinazoline, and quinoxaline. A heteroaryl group may be a fused polycyclic ring systems, including for instance fused bicyclic systems in which a heteroaryl group is fused to an aryl group as defined herein.

As well as being substituted with a reducible nitrogen-containing functional group as defined herein, further substituents may be present as discussed above.

In some embodiments, the choice of oxidoreductase enzyme is determined by the reduction potential of the target compound. In some embodiments the oxidoreductase enzyme is capable of oxidising the molecular reductant at a potential more negative than the reduction potential of the target compound. It is straight forward to determine the reduction potential of a target compound, e.g. by using electrochemistry as described in the examples. Similarly, it is straight forward to determine the potential at which an oxidoreductase enzyme is capable of oxidising a given molecular reductant. For example, protein film voltammetry can be used. In brief, the oxidoreductase enzyme is immobilised on an electrode optionally comprising a support material as described herein in the presence of the molecular reductant and the voltage potential applied to the electrode is altered, with a current corresponding to oxidation of the reductant being observed at potentials at which the oxidoreductase enzyme is capable of oxidising the molecular reductant.

Typically, the nitrogen-containing functional group is attached to a non-saturated carbon atom. Often the target compound comprises an aromatic nitrogen-containing functional group. In other words, the nitrogen-containing functional group is typically attached to an aromatic (hydrocarbyl aromatic or heteroaromatic) group. More typically the target compound comprises an aromatic nitro group; an aromatic azide group or an aromatic nitrile group. For example the target compound may be a nitroaromatic compound. In some embodiments the nitrogen-containing functional group is covalently attached to an aromatic group which is further meta or para substituted relative to the nitrogen-containing functional group. In some embodiments the nitrogen-containing functional group is covalently attached to an aromatic group which is ortho substituted relative to the nitrogen-containing functional group. In some embodiments the nitrogen-containing functional group is covalently attached to an aromatic group which is not ortho substituted relative to the nitrogen-containing functional group. In some embodiments the nitrogen-containing functional group is covalently attached to an aromatic group which is not ortho substituted relative to the nitrogen-containing functional group with a Bu group.

In some embodiments the target compound comprises multiple functional groups, for example the target compound may comprise a reducible nitrogen-containing functional group and a reducible non-nitrogenous functional group such as a C═C double bond. Typically, the methods provided herein are capable of selectively reducing the nitrogen-containing functional group without reducing the non-nitrogenous functional group. Without being bound by theory, the inventors believe that reducible nitrogen-containing functional groups may have appropriate reduction potentials to be reduced a supported biocatalyst comprising a oxidoreductase enzyme as described herein.

As will thus be apparent, the disclosed methods comprise the formation of a reaction product comprising a reduced functional group, e.g. comprising a reduced nitrogen-containing functional group as described herein. Accordingly, also provided herein is a method of forming a reaction product comprising a reduced nitrogen-containing functional group, the method comprising contacting a target compound comprising an oxidised nitrogen-containing functional group with a supported biocatalyst comprising an oxidoreductase enzyme supported on a support material as described herein; in the presence of a molecular reductant as described herein, under conditions such that:
the molecular reductant is oxidised by the oxidoreductase enzyme or functional fragment or derivative thereof; and
the nitrogen-containing functional group is reduced at the support material;
thereby forming the reaction product.

Typically, the reaction product comprises an aromatic amine, i.e. an amine or quaternary ammonium group bonded to a hydrocarbyl aromatic or heteroaromatic group as defined herein.

### Preferred Aspects

In some preferred embodiments, the disclosed method is a method of reducing a reducible nitrogen-containing functional group in a target compound; wherein the nitrogen-containing functional group is selected from an nitro group, an azide group, a hydroxylamine group, a nitrile group, a diazo group and an isocyanide group; and wherein said reduction converts said nitrogen-containing functional group to an amine group;
the method comprising contacting the target compound with a biocatalyst; wherein said biocatalyst comprises an oxidoreductase enzyme selected from a hydrogenase and/or a carbon monoxide dehydrogenase and/or a formate dehydrogenase or a functional fragment or derivative thereof supported on a conductive or semi-conductive support material,
in the presence of a molecular reductant selected from molecular hydrogen and/or CO and/or formate, under conditions such that:
   the molecular reductant is oxidised by the oxidoreductase enzyme or functional fragment or derivative thereof; and
   the nitrogen-containing functional group is reduced to an amine group at the support material.

In some further preferred embodiments, the disclosed method is a method of reducing a reducible nitrogen-containing functional group in a target compound; wherein the nitrogen-containing functional group is selected from a nitro group, an azide group, or a nitrile group; and wherein said reduction converts said nitrogen-containing functional group to an amine group;
the method comprising contacting the target compound with a biocatalyst; wherein said biocatalyst comprises a hydrogenase enzyme or a functional fragment or derivative thereof supported on a conductive or semi-conductive support material, wherein said support material comprises carbon, a metal or metal alloy, a metal oxide or mixed metal oxide, a metal hydroxide, a metal chalcogenide, a semi-conducting material, or an electronically-conductive polymer, or mixtures thereof;
in the presence of molecular hydrogen or an isotope thereof; under conditions such that:
   the hydrogen or isotope thereof is oxidised by the hydrogenase or functional fragment or derivative thereof; and
   the nitrogen-containing functional group is reduced to an amine group at the support material.

In still further preferred embodiments, the disclosed method is a method of reducing a nitro group in a nitroaromatic target compound; wherein said reduction converts said nitro group to an amine group;
the method comprising contacting the nitroaromatic target compound with a biocatalyst; wherein said biocatalyst comprises a hydrogenase enzyme or a functional fragment or derivative thereof supported on carbon-based support material;
in the presence of molecular hydrogen or an isotope thereof; under conditions such that:
   the hydrogen or isotope thereof is oxidised by the hydrogenase or functional fragment or derivative thereof; and
   the nitrogen-containing functional group is reduced to an amine group at the support material.

### Reaction Conditions

In the disclosed methods, the target compound is preferably initially added to or present in the reaction medium at a concentration of 1 μM to 1 M, such as from 5 μM to 800 mM, e.g. from 10 μM to 600 mM such as from 25 μM to 400 mM e.g. from 50 μM to 200 mM such as from 100 μM to about 100 mM e.g. from about 250 μM to about 10 mM such as from about 500 μM to about 1 mM.

As explained above, when the molecular reductant is a gas such as hydrogen or CO, the disclosed methods are typically conducted under a gas atmosphere; i.e. in the presence of gas (for example in the headspace of a reactor). Preferably, the gas atmosphere comprises hydrogen or an isotope thereof and/or CO or an isotope thereof and optionally an inert gas. O₂ or an isotope thereof may be present. Preferred inert gases include nitrogen, argon, helium, neon, krypton, xenon, radon and sulfur hexafluoride (SF₆) and mixtures thereof, more preferably nitrogen and/or argon, most preferably nitrogen.

When the gas atmosphere comprises a mixture of hydrogen and an inert gas and/or O₂, the hydrogen is preferably present at a concentration of 1-100%, with the remaining gas comprising an inert gas as defined herein and/or O₂. Preferred gas atmospheres include from 80-100% H₂ with the remaining gas comprising one or more inert gases; and from 0-20% H₂ with the remaining gas comprising one or more inert gases and/or O₂ (such as from 1-4% H₂ in air). When the gas atmosphere comprises a mixture of CO and an inert gas and/or O₂, the CO is preferably present at a concentration of 1-100%, with the remaining gas comprising an inert gas as defined herein and/or O₂. Preferred gas atmospheres include from 80-100% CO with the remaining gas comprising one or more inert gases; and from 0-20% CO with the remaining gas comprising one or more inert gases and/or O₂ (such as from 1-4% CO in air).

The gas atmosphere may optionally also include non-inert gases such as ammonia, carbon dioxide and hydrogen sulphide. Preferably, however, the gas atmosphere is free of ammonia, carbon dioxide and hydrogen sulphide. The methods of the invention may be conducted at any suitable pressure: selecting an appropriate pressure is an operational parameter of the methods of the invention which can be controlled by the operator. Sometimes, the methods of the invention are conducted at ambient pressure (e.g. about 1 bar). Sometimes, the methods of the invention are conducted at reduced pressure (e.g. less than 1 bar) or at elevated pressure (e.g. greater than 1 bar). For example, increasing the operating pressure can increase hydrogen solubility in the reaction medium. Preferably, the methods of the invention are carried out at a pressure of from about 0.1 bar to about 20 bar, such as from about 1 bar to about 10 bar, e.g. from about 2 bar to about 8 bar such as from about 4 bar to about 6 bar, e.g. about 5 bar.

The disclosed methods may be carried out under aerobic or anaerobic conditions. As used herein, “aerobic conditions” refers to the gas atmosphere not being strictly anaerobic, e.g. comprising at least trace O₂. Suitable O₂ levels are typically greater than 100 ppm, e.g. greater than 1000 ppm (0.1%), such as greater than 1% O₂, for example greater than 2% O₂. Usually, O₂ levels do not exceed the O₂ levels in atmospheric air, i.e. 21% O₂, however greater O₂ levels are not excluded.

The provided methods are typically conducted in an aqueous composition which may optionally comprise e.g. buffer salts. For some applications buffers are not required and the methods of the invention can be conducted without any buffering agents. Preferred buffer salts which can be used in the methods of the invention include Tris; phosphate; citric acid/Na₂HPO₄; citric acid/sodium citrate; sodium acetate/acetic acid; Na₂HPO₄/NaH₂PO₄; imidazole (glyoxaline)/HCl; sodium carbonate/sodium bicarbonate; ammonium carbonate/ammonium bicarbonate; MES; Bis-Tris; ADA; aces; PIPES; MOPSO; Bis-Tris Propane; BES; MOPS; TES; HEPES; DIPSO; MOBS; TAPSO; Trizma; HEPPSO; POPSO; TEA; EPPS; Tricine; Gly-Gly; Bicine; HEPBS; TAPS; AMPD; TABS; AMPSO; CHES; CAPSO; AMP; CAPS and CABS. Selection of appropriate buffers for a desired pH is routine to those skilled in the art, and guidance is available at e.g. http://www.sigmaaldrich.com/life-science/core-bioreagents/biological-buffers/learning-center/buffer-reference-center.html. Buffer salts are preferably used at concentrations of from 1 mM to 1 M, preferably from 10 mM to 100 mM such as about 50 mM in solution. Most preferred buffers for use in methods of the invention include 50 mM phosphate, pH 8.0.

The provided methods are typically conducted in an aqueous composition. However, non-aqueous components can optionally be used instead or as well as water in the compositions used in the disclosed methods. For example, one or more organic solvents (e.g. alcohols, DMSO, acetonitrile, etc) or one or more ionic liquids may be used or included in the compositions.

The disclosed methods may be carried out in a mixed solvent system comprising a plurality (e.g. 2, 3, 4, 5 or more) of aqueous or non-aqueous solvents, e.g. solvents as described herein. In some embodiments the disclosed methods are carried out in a monophasic solvent system. In some embodiments the disclosed methods are carried out in a non-miscible solvent mixture such as a biphasic or triphasic solvent system.

The disclosed methods are typically carried out at a temperature of from about 10° C. to about 80°, such as from about 20° C. to about 60° C., e.g. from about 30° C. to about 50° C.

The disclosed methods are typically carried out at a pH of from about pH 4 to about pH 10 such as from about pH 5 to about pH 9 e.g. from about pH 6 to about pH 8 e.g. about pH 7.

The disclosed methods may be performed in an apparatus as provided herein. The apparatus typically comprises a reaction vessel. The reaction vessel typically comprises one or more inlets for molecular hydrogen gas or hydrogen-containing liquids (e.g. hydrogen saturated liquids such as buffer solutions as described herein); and/or one or more inlet for reagents; and/or one or more outlets for product. Further equipment such a pressure controls, temperature controls, mixing apparatus, flow controls, etc may be incorporated. The apparatus may be comprised as a part of an apparatus for converting initial reagents into final products and thus be configured to perform an intermediate reaction step. The apparatus may be controlled by equipment such as a computer controller. The apparatus may comprise means for detecting cofactor turnover, reagent utilisation and/or product production, e.g. spectrophotometric means. The apparatus may be configured to be operated in flow mode (i.e. continuous mode) or in batch mode.

Accordingly, the methods provided herein may be performed in a flow setup, e.g. in a flow reaction cell. The methods provided herein may alternatively be performed in a batch setup e.g. in a batch reaction cell.

### System

Also provided herein is a system for performing a method as disclosed herein.

Preferably, the system comprises:
i) a biocatalyst comprising an oxidoreductase enzyme or a functional fragment or derivative thereof supported on a support material;
ii) a molecular reductant; and
iii) a target compound comprising a reducible nitrogen-containing functional group;
wherein the system is configured such that, in use, (a) the molecular reductant is oxidised by the oxidoreductase enzyme or functional fragment or derivative thereof; and (b) the nitrogen-containing functional group is reduced at the support material.

Typically, said system is designed or configured such that the oxidoreductase enzyme or functional fragment or derivative transfers electrons to the support material and the reduction of the nitrogen-containing functional group comprises direct electron transfer from the support material to the target compound, e.g to the reducible functional group of the target compound.

In the systems provided herein, the oxidoreductase enzyme is typically as defined herein. The support material is typically as defined herein. The molecular reductant is typically as defined herein. The target compound and functional group comprised therein is typically as defined herein. The system may be configured to be operated as described for the methods provided herein. The system may be configured to be operated under reaction conditions as defined herein.

Typically, the system may further comprise means for controlling a concentration of molecular reductant present in the reaction medium (e.g. by controlling the gas atmosphere in the system, e.g. by using a gas flow system). The system is often configured as a flow cell containing reagents as described herein. The system (e.g. a flow cell) may comprise features of the provided apparatus described herein, such as one or more inlets for the molecular reductant (e.g. hydrogen or CO gas or formate) and/or one or more inlet for reagents; and/or one or more outlets for product; and/or one or more pressure controls, temperature controls, mixing apparatus, flow controls, etc

The following Examples illustrate the invention. They do not, however, limit the invention in any way. In this regard, it is important to understand that the particular assays used in the Examples section are designed only to provide an indication of the efficacy of the method of the invention. There are many assays available to determine reaction efficiency, and a negative result in any one particular assay is therefore not determinative.

### EXAMPLES

### Example 1

This example demonstrates the complete reduction of reducible nitrogen-containing functional groups in accordance with the disclosed methods.

A hydrogen-oxidising oxidoreductase enzyme E. coli hydrogenase 1 (Hyd1) was expressed, purified and immobilized onto a support material as described herein. The support material used was carbon black nanopowder (BP2000, Cabot corp). Hyd1 is a good H₂ oxidiser (H₂→2H⁺+2e⁻) and is O₂-tolerant.¹ A control experiment used an equivalent quantity of Hyd1 in solution without the support material. The reduction of an exemplary nitrogen-containing functional group (the nitro group of 2-methyl-5-nitropyridine, compound A, was quantified by 1H NMR spectroscopy (FIG. 10)). Reaction conditions: 0.25 mL reaction contained 5 mM A in Tris-HCl (50 mM, pH 8.0) with 2 vol % DMSO under 1 bar H₂.

Results are shown in Table 1. Hyd1 (6 μL) immobilised by adsorption on 0.5 mg of the carbon support material catalysed the complete 6-electron reduction of the nitro group of A to the amine analogue B (2-methyl-5-aminopyridine) with 100% conversion after 6 hours. By contrast, in the absence of any support material reduction of the nitro group was inefficient with 71% of the target compound A remaining in the oxidised form after 2 hours and 40 min, even at an elevated temperature of 30° C. No complete reduction was observed, with the 21% of the target compound which was reduced forming the hydroxylamine compound C (2-methyl-5-hydroxylaminopyridine).

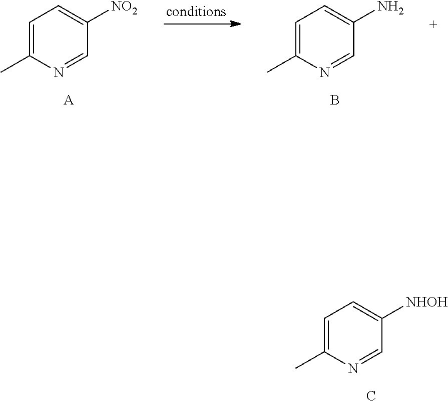

TABLE 1

| | Relative compound distribution | | |
|---|---|---|---|
| Catalyst | A | B | C |
| Hyd1 in solution (18.5 μg), 30° C. | 71% | 0% | 21% |
| Hyd1/C, (22 μg on 0.5 mg C), 22° C. | 0% | 100% | 0% |

This example thus demonstrates that the presence of a support material as described herein leads to improvements in nitrogen-group reduction, in terms both of leading to more complete reduction and to greater reduction efficiency. Without being bound by theory, the inventors believe that the support material when modified with an oxidoreductase enzyme such as a hydrogenase such as Hyd1 serves as an “active site” for promoting the complete reduction of nitrogen-containing functional groups. Without being bound by theory, the inventors propose that the mechanism for nitro reduction occurs via a series of electron and/or hydride transfers from the carbon material to the nitro group.

### Example 2

Example 1 was repeated with other target compound comprising nitrogen-containing functional groups: 4-nitrophenol (i) and 1-methyl-2-nitrobenzene (ii); see Scheme 1. Again the supported biocatalyst was capable of the efficient and complete reduction of the nitro group, demonstrating the chemical versatility of the disclosed methods.

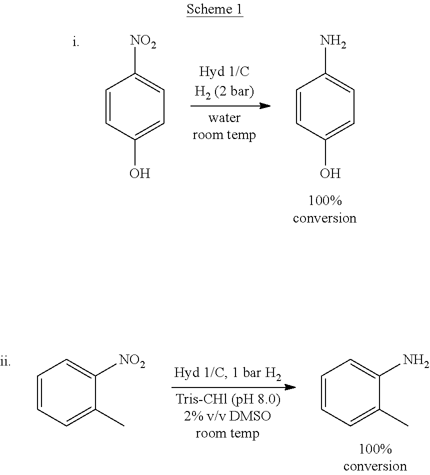

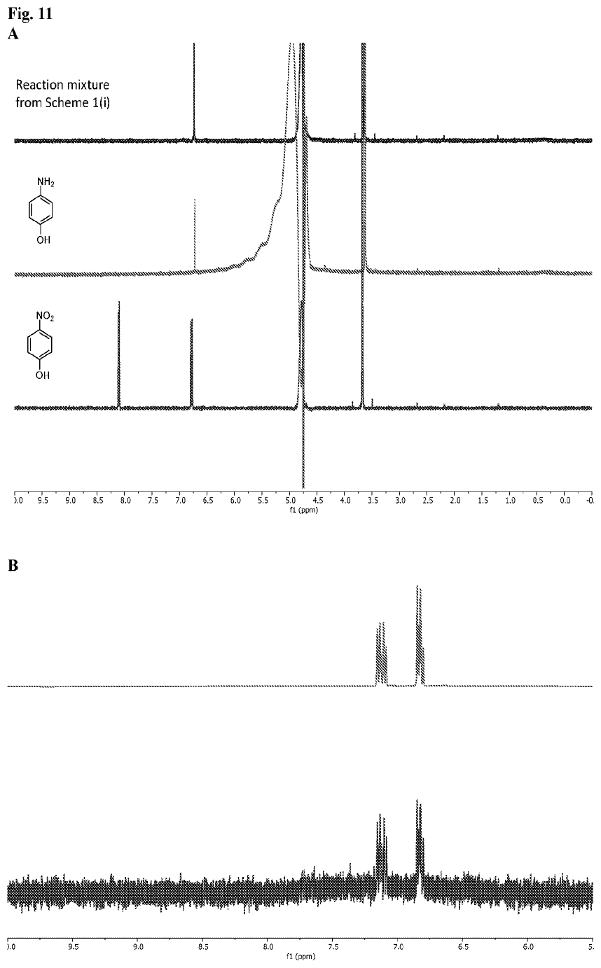

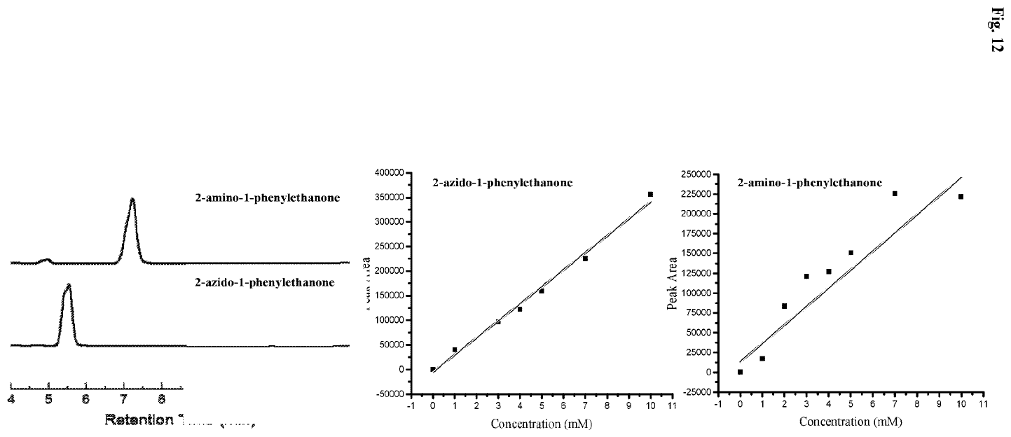

Reaction efficiency was determined by ¹H NMR (FIG. 11). Optimal reduction efficiency was observed for reaction (i) at pH 7, with the reaction proceeding to completion after 21 hours.

### Example 3

E. coli Hyd-1-modified carbon particles were packed into a packed-bed flow column as follows: A stainless steel flow cartridge (30×4 mm) was packed with 50 mg activated charcoal using a cartridge packer (ThalesNano). A 0.2 mL (1.9 mg) solution of Hyd1 was injected into the cartridge before it was sealed. The cartridge was sealed using the cartridge packer, and was agitated on a vortex mixer and then stored at 4° C. for 1 h. The cartridge was then stored at 4° C. for 1 h, then installed in a reactor (H-Cube) and flushed with sodium phosphate buffer (100 mM, pH 6.0) to remove any unadsorbed enzyme.

Reaction (i) of Example 2 was repeated under flow conditions as follows: a liquid feed (Knauer H-Cube piston pump) containing 10 mM 4-nitrophenol in sodium phosphate buffer (100 mM, pH 6.0) was pumped through the H-Cube with the H₂ that was generated by built-in water reservoir and electrolyzer (gas feed pressure was set to 2 bar H₂). The liquid feed flow rate was set to 0.1 ml per min to give a 1.6 s residence time (tRes) through the biocatalyst cartridge. The reaction was run at 28° C. Aliquots were removed periodically for analysis by UV-vis spectroscopy and HPLC.

Successful complete reduction of the nitro group to the corresponding amine group as depicted in scheme 1 was observed under industrially relevant conditions. The reaction ran at high (˜80%) conversion for ˜92 h. This aligns with Hyd1 TTN>1.4 million.

### Example 4

Example 1 was repeated with a different model target compound having an aliphatic azide functional group rather than a heteroaromatic nitro group. The target compound was 2-azido-1-phenylethanone (1 below). Reaction conditions were 5 mM of the target compound (1) in buffer and cosolvent at room temp, mixed on a shaker plate under a steady flow of H₂. Conversions were determined using HPLC after the reaction mixture was subjected to Boc-derivitisation conditions (see caption, FIG. 12). As shown in Table 2, close to 100% conversion was observed after 21 hours.

Additional control experiments were performed to confirm the role of the oxidoreductase enzyme and the support material in the supported biocatalyst. No reduction of the azide group was observed in control experiments in which (i) the carbon support material was omitted, with an equivalent amount of E. coli Hyd1 in solution being used; (ii) the oxidoreductase enzyme was omitted, with an equivalent amount of carbon support material being used; or (iii) the carbon support material and hydrogenase were both omitted.

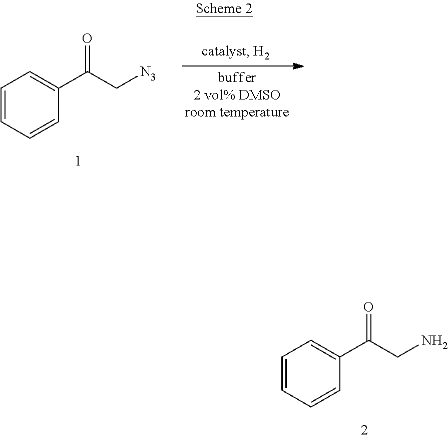 [SMILES: [N-]=[N+]=NCC(=O)c1ccccc1 ; NCC(=O)c1ccccc1]

TABLE 2

| | | | Conversion |
|---|---|---|---|
| Catalyst | Buffer | pH | (%) |
| Hyd1/C | Tris-HCl | 8 | 98 |
| Hyd1 (=E. coli Hyd1 in solution; | Tris-HCl | 8 | 0 |
| no carbon support material) | | | |
| Carbon particles only; no enzyme | Tris-HCl | 8 | 0 |
| none | Tris-HCl | 8 | 0 |

### Example 5

Nitrogen-containing functional groups of further target compounds were reduced using the system of Example 1. This example demonstrates (i) that the disclosed methods can be used to reduce aryl azides with high efficiency and selectivity; (ii) that further substitution on the aryl group is well tolerated; and (iii) that unwanted side-reactions such as dehalogenations of para-chloro substituents are avoided. Such unwanted side-reactions are a common problem when using precious metal catalysts such as carbon-supported palladium (Pd/C) are used to catalyse reductions of aryl nitrogen-containing functional groups such as azides.^{2,3}

Results are shown in Table 3. Reaction conditions: 0.25 mL reaction volume, 5 mM azide (target compound), 19 ug Hyd1 enzyme immobilized on 25 ug carbon black nanoparticles support material, mixed under a 1 bar atmosphere of H₂.

TABLE 3

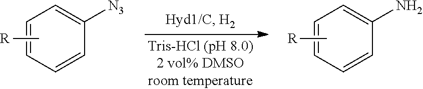

| Entry | R | Conversion |
|---|---|---|
| 1 | H | 100% |
| 2 | o-CH₃ | 95% |
| 3 | o-CF₃ | 76% |
| 4 | m-CH₃ | 91% |
| 5 | p-OCH₃ | 81% |
| 6 | p-CN | 72% |
| 7 | p-Cl | 94%* |

*no dehalogenated product detected

Conversions were determined using GC-FID. Conversions were calculated by comparing the peak areas of the azide and corresponding aniline.
GC-FID method:
Column: CP-Chirasil-Dex CB (Agilent), 25 m length, 0.25 mm diameter, 0.25 μm (film thickness), fitted with a guard of 10 m undeactivated fused silica of the same diameter
Carrier: He (CP grade), 170 kPa (constant pressure)
Inlet temperature: 200° C.
Injection conditions: Splitless with split flow 60 mL/min, splitless time 0.8 mins, purge 5 mL/min. Injection volume=0.5 μL.
Detection: FID (H₂=35 mL/min, air=350 mL/min, makeup N₂=40 mL/min, temp=200° C.)
Oven heating profile:

| Time (minutes) | Temperature |
|---|---|
| 0 → 5 | Hold at 70° C. |
| 5 → 30 | Ramp to 120° C. at 2° C./min |
| 30 → 36 | Ramp to 180° C. at 10° C./min |
| 36 → 45 | Hold at 180° C. for 5 minutes |

### Compound Retention Times:

| | Reten- | | Reten- |
|---|---|---|---|
| | tion | | tion |
| | time | | time |
| Azide | (min) | Amine | (min) |
| Phenyl azide | 7.78 | Aniline | 10.02 |
| 1-Azido-2-methylbenzene | 9.36 | o-Toluidine | 11.19 |
| 1-Azido-2- | 8.47 | 2-(Trifluoromethyl)- | 9.37 |
| (trifluoromethyl)benzene | | aniline | |
| 1-Azido-3-methylbenzene | 9.31 | m-Toluidine | 11.19 |
| 1-Azido-4-methoxybenzene | 12.42 | 4-Methoxyaniline | 13.06 |
| 4-Azidobenzonitrile | 14.29 | 4-Amino-benzonitrile | 16.64 |
| 1-Azido-4-chlorobenzene | 11.38 | 4-Chloroaniline | 13.45 |

### Example 6

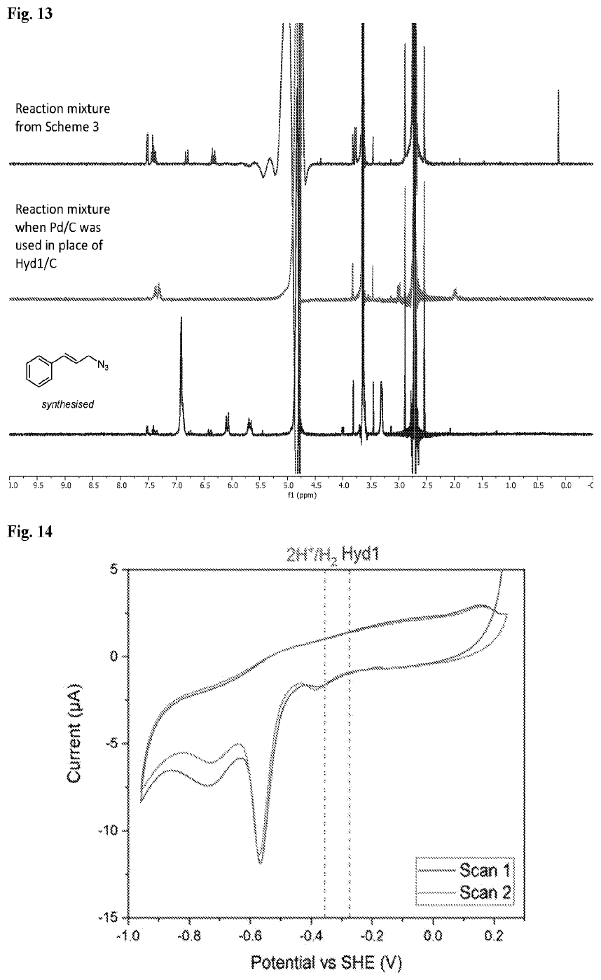

The selectivity of the disclosed methods in reducing nitrogen-containing functional groups compared to other reducible-functional groups such as unsaturated C═C double bonds was demonstrated by repeating Example 1 using cinnamyl azide (3). Product distributions were determined using ¹H NMR spectroscopy (See FIG. 13).

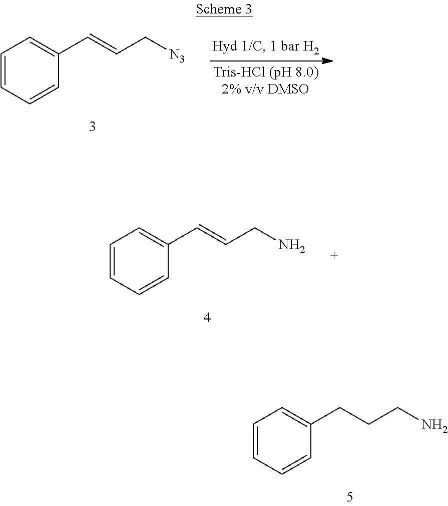 [SMILES: [N-]=[N+]=NC/C=C/c1ccccc1 ; NC/C=C/c1ccccc1 ; NCCCc1ccccc1]

Relative compound distribution

| Catalyst | 4 | 5 |
|---|---|---|
| Hyd1/C | 100% | 0% |
| Pd/C | 0% | 100% |

A control experiment was performed using a conventional precious metal catalyst (Pd/C) under identical reaction conditions. Both the alkene group and azide group of compound 3 were reduced to form 5.

The lack of peaks in the alkene region (i.e. 5-7 ppm) of the spectrum showing the product of the reaction with Pd/C as the catalyst confirms that Pd/C reduced the alkene. By contrast, the spectrum for the Hyd1/C-catalysed reaction contains alkene peaks downshifted relative to the starting azide, demonstrating formation of the corresponding cinnamyl amine.

This example thus demonstrates the improved chemoselectivity of the disclosed systems and methods compared to conventional catalysts in selectively reducing nitrogen-containing functional groups.

### Example 7

Experiments were conducted to probe the mechanism and product distribution of the reactions described in examples 1 to 6.

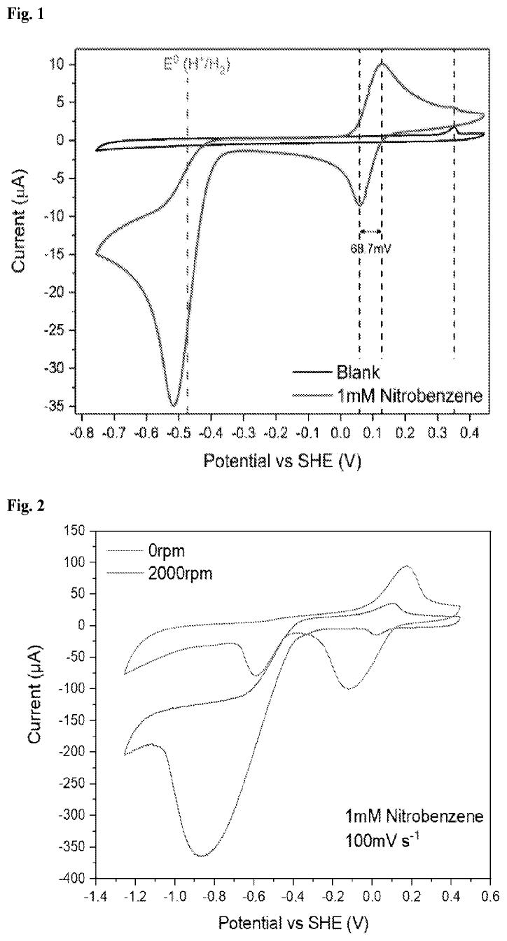

FIG. 1 shows the electrochemistry of 1 mM nitrobenzene in pH 8 buffer at a pyrolytic graphite edge (PGE) electrode. The reductive wave at more negative potentials shows the irreversible reduction of the nitro-group to an amino-group. FIG. 1 shows that the onset potential for the reduction of nitrobenzene to aniline is significantly more positive than the hydrogen couple (assuming pH 8 and 1 bar H₂). This confirms that electrons provided by a hydrogenase oxidising dihydrogen could be used by PGE to reduce nitrobenzene to aniline.

FIG. 2 confirms that, like PGE, carbon black nanopowder (BP2000) is able to reduce nitrobenzene at potentials more positive than the hydrogen couple under the mentioned conditions.

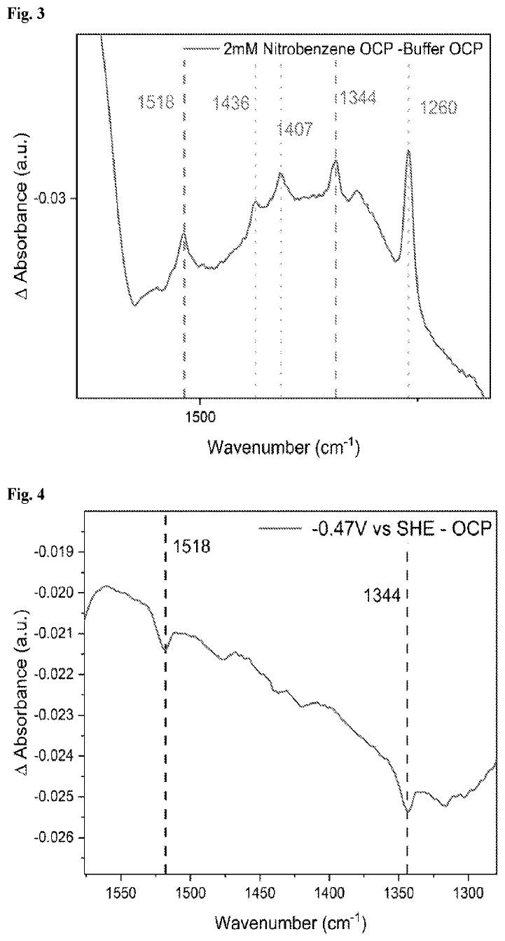

FIG. 3 shows a IR difference spectrum of 2 mM nitrobenzene in 1 vol % DMSO in 50 mM tris-HCl showing two N—O stretch absorption bands of the nitro group (dark grey dashed lines) and three bands originating from DMSO (light grey dotted lines). FIG. 4 shows that when a reducing potential equal to the potential of the hydrogen couple is applied to the BP working electrode, the N—O stretch bands (black dashed lines) are disappearing. This indicates that the reductive wave starting around−0.4V vs SHE corresponds to the reduction of the nitro group of nitrobenzene.

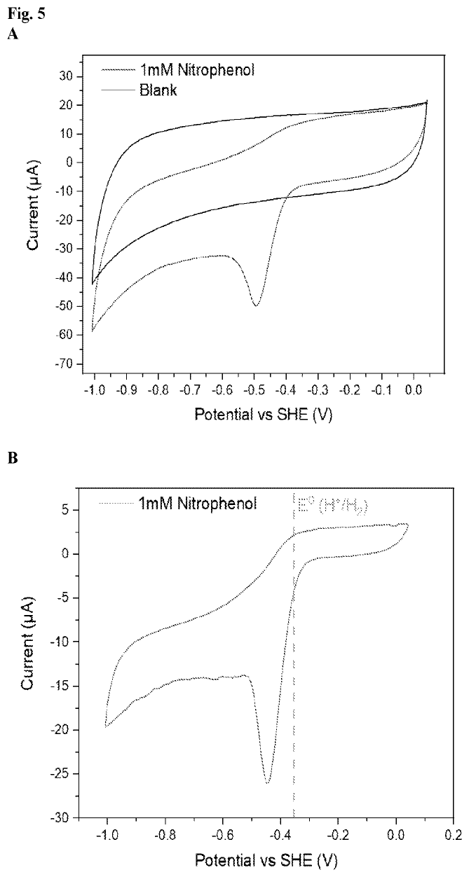

The electrochemical behaviour of nitrophenol in aqueous electrolyte (100 mM sodium phosphate pH 6) on BP electrodes was also assessed (FIG. 5A). An irreversible reductive wave starting around −0.3V vs SHE is clearly observed. When scanning the potential at a lower rate (i.e. closer to steady-state conditions, FIG. 5B) it is clear that the reductive wave starts at potentials significantly more positive than the hydrogen couple under these conditions (pH 6, 1 bar hydrogen equals Eº (H⁺/H₂)=−0.355V vs SHE). This again indicates that it is possible to reduce nitrophenol on a BP surface with electrons supplied by a H₂-oxidising hydrogenase such as Hyd1.

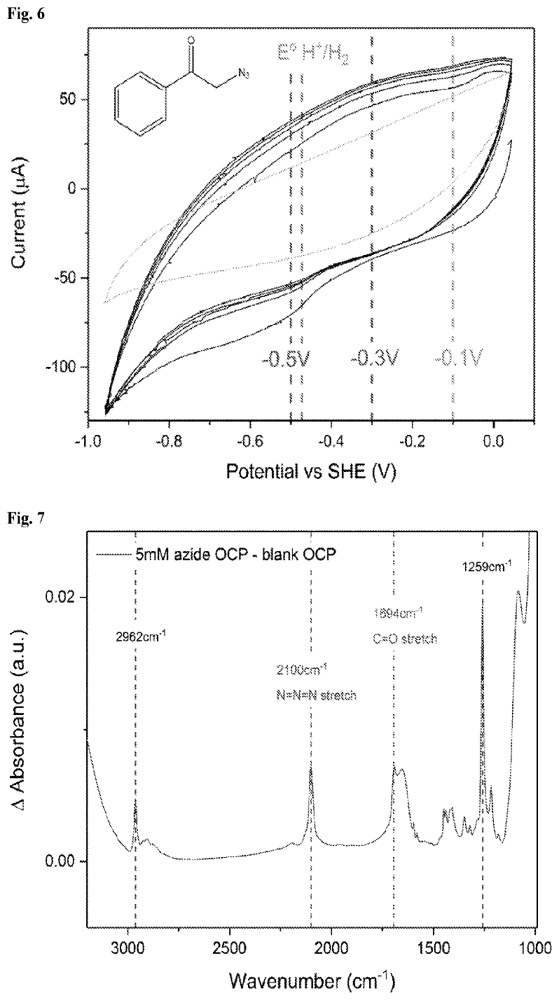

The electrochemistry of 5 mM 2-azido-1-phenylethanone on a BP electrode was further investigated in a spectroelectrochemical cell. A reductive wave at potentials below −0.4V vs SHE can be observed in cyclic voltammograms (FIG. 6). Similar to the nitrobenzene and nitrophenol case, this is significantly more positive then the hydrogen couple under these conditions (pH 8, 1 bar hydrogen equals Eº (H⁺/H₂)=−0.473V vs SHE) again indicating that it is possible to reduce 2-azido-1-phenylethanone on a BP surface with electrons supplied by a H₂-oxidising hydrogenase such as Hyd1.

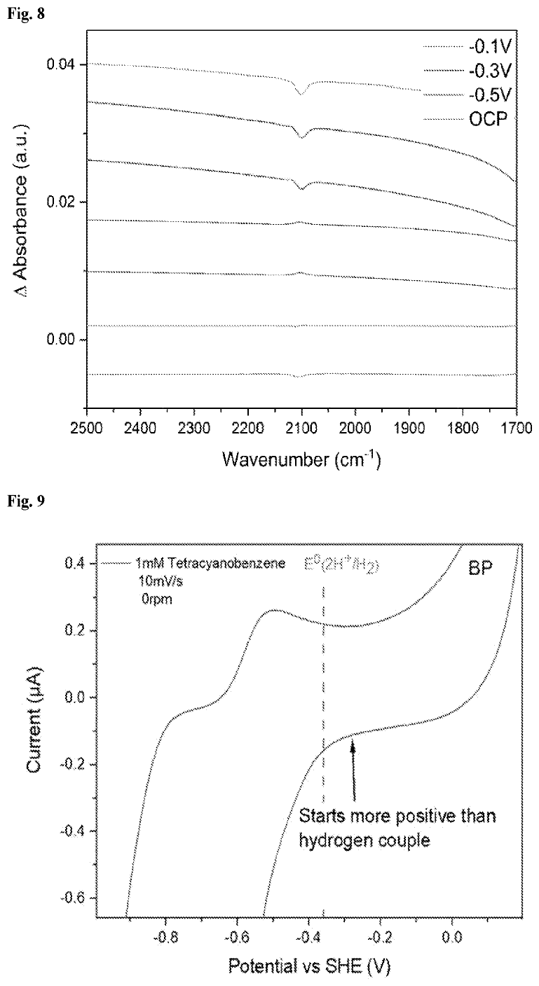

FIG. 7 shows a IR difference spectrum of 5 mM 2-azido-1-phenylethanone in 1 vol % DMSO in 50 mM tris-HCl showing the N═N═N stretch absorption band of the azide group (red line) as well as the C═O stretch of the carbonyl group (blue line) of the 2-azido-1-phenylethanone. FIG. 8 shows a IR difference spectra of 5 mM 2-azido-1phenylethanone in 50 mM tris-HCl pH 8 with 1 vol % DMSO at different potentials (vs SHE) applied to a BP-modified activated carbon paper working electrode. FIG. 8 shows that when a reducing potential roughly equal to the potential of the hydrogen couple is applied to the BP working electrode, the N═N═N stretch band (around 2100 cm¹) is disappearing. This indicates that the reductive wave starting around −0.4V vs SHE is indeed the reduction of the azide group of 2-azido-1-phenylethanone and confirms that BP can use electrons supplied by a H₂-oxidising hydrogenase to reduce azide groups.

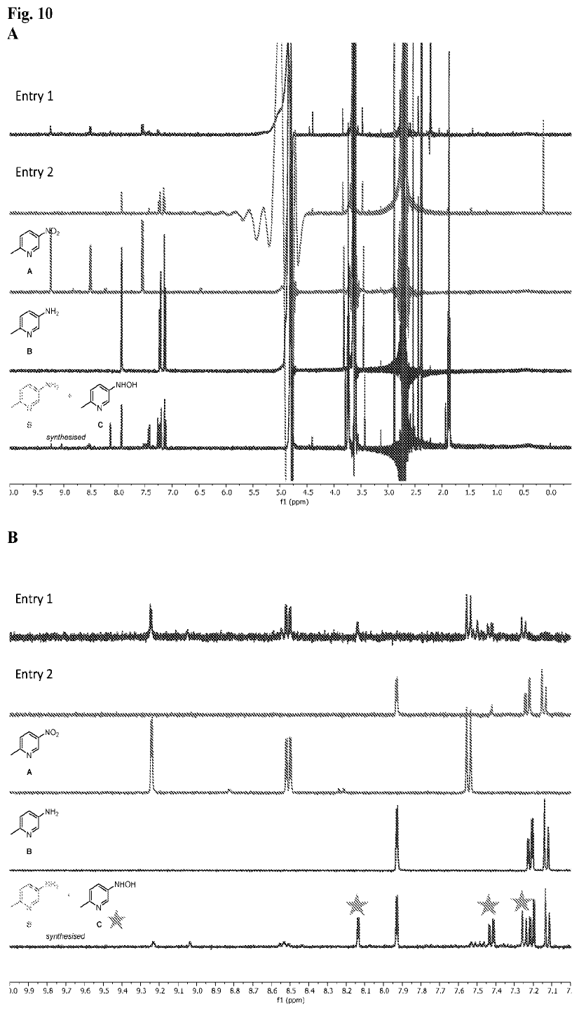

FIG. 9 shows a zoomed-in region of a cyclic voltammogram of a PGE electrode modified with BP200 in 100 mM sodium phosphate pH 6 with 1 vol % DMSO and 1 mM tetracyanobenzene at a rotation rate of 0 rpm obtained with a scan rate of 10 mV s⁻¹. This shows that the tetracyanobenzene can reduce at the carbon surface, with reduction wave starting at potentials slightly more positive than the hydrogen couple at pH 6 (assuming H₂ pressure is 4 bar). This indicates that the supported biocatalyst described herein (e.g. hydrogenase on carbon) can reduce the nitrile substrate.

### Example 8

Further experiments to demonstrate the broad applicability of the disclosed methods were conducted. As shown in Table 4, a wide range of target compounds were successfully addressed by controlling the reaction conditions. Selection of suitable reaction conditions is well within the capacity of those skilled in the art and the choice of reaction conditions in order to result in a desired outcome is an operational parameter of the method which can be controlled by the skilled user.

Reaction conditions: 10 mM substrate in 100 mM sodium phosphate buffer, pH 6 (unless indicated otherwise) and cosolvent at room temperature, mixed on a shaker plate under constant pressure of 2 bar H₂ in pressure vessel for 24 h.

TABLE 4

| | | Acetonitrile | Conver- |
|---|---|---|---|
| Entry | Substrate | (vol %) | sion |
| 1 | 4-nitrophenol (pH 5) | 0 | 45% |
| 2 | 4-nitrophenol (pH 6) | 0 | 92% |
| 3 | 4-nitrophenol (pH 7) | 0 | 80% |
| 4 | 4-nitrophenol (pH 8) | 0 | 38% |
| 5 | 2-bromo-4-nitrophenol | 0 | 74% |
| 6 | 2-fluoro-4-nitrophenol | 0 | 99% |
| 7 | 2-methyl-4-nitrophenol | 10 | 43% |
| 8 | 4-nitrothioanisole | 20 | 26% |
| 9^{a} | 4′-chloro-3-nitro-1,1′-biphenyl | 10 | 94% |
| | (pH 8) | | |
| 10^{a} | 4′-chloro-2-nitro-1,1′-biphenyl | 10 | 92% |
| | (pH 8) | | |
| 11 | 3-nitrostyrene | 10 | ✓^{b} |

^{a}Entry 9 & 10 were carried out in 50 mM tris-HCl pH 8.
No side products were identified except for ^{b}where conversion to 3-vinylaniline was observed, as well as another byproduct.
Conversions were determined using HPLC.

### HPLC Method:

Column: Shim-pack GIS 5 μm HILIC Reverse Phase Column (Shimadzu), 150 mm length, 4.6 mm diameter, fitted with a 5 μmShim-pack GIS(G) guard column of 4 mm length and 10 mm diameter
Mobile Phase: 80% 20 mM ammonium acetate buffer pH 7.7, 20% acetonitrile at flow rate of 1 ml/min
Oven temperature: 40° C.
Injection conditions: Injection volume=10 μL.
Detection: UV at 260 and 310 nm

| | Retention | | Retention |
|---|---|---|---|
| Nitro Substrate | time (min) | Amine Product | time (min) |
| 4-Nitrophenol | 3.4 | 4-Aminophenol | 2.7 |
| 2-bromo-4-nitrophenol | 2.6 | 2-bromo-4-aminophenol | 3.9 |
| 2-fluoro-4-nitrophenol | 2.2 | 2-fluoro-4-aminophenol | 2.8 |
| 2-methyl-4-nitrophenol | 4.2 | 2-methyl-4-aminophenol | 3.0 |
| 4-nitrothioanisole | 6.0 | 4-aminothioanisole | 4.4 |
| 4′-chloro-3-nitro- | 2.6 | 4′-chloro-3-amino- | 2.9 |
| 1,1′-biphenyl | | 1,1′-biphenyl | |
| 4′-chloro-2-nitro- | 2.6 | 4′-chloro-2-amino- | 3.0 |
| 1,1′-biphenyl | | 1,1′-biphenyl | |
| 3-nitrostyrene | 5.8 | 3-aminostyrene | 4.7 |

### Example 9

This example describes various nitro group reductions using a catalyst ‘CaHydA1/C’ formed by immobilising C. acetobutylicum [FeFe]-hydrogenase Al (CaHydA1) onto carbon black nanopowder (BP2000). CaHydA1 is a good H₂ oxidiser that operates without an overpotential and as such can provide a large driving force.

Table 5 shows the successful conversion of various substituents using CaHydAl/C. No side products were identified.

Reaction conditions: 10 mM substrate in 100 mM sodium phosphate buffer, pH 6, and cosolvent as indicated, were mixed at room temperature on a shaker plate under constant pressure of 2 bar H₂ in pressure vessel for 24 h.

TABLE 5

| | Acetonitrile | |
|---|---|---|
| Substrate | (vol %) | Conversion |
| 4-nitrophenol | 0 | 87% |
| 2-methyl-4-nitrophenol | 10 | 82% |
| 1-nitrohexane | 10 | ✓ |
| 1-nitropentane | 10 | ✓ |

### Example 10

Some compounds having reducible nitrogen-containing functional groups have reduction potentials which are challenging to access. By using oxidoreductase enzymes which operate at low overpotentials, such compounds can be readily reduced in accordance with the methods disclosed herein.

Examples of such compounds include 1-nitrohexane and 1-nitropentane. As shown in FIG. 14, these compounds have reduction potentials only slightly more positive than the hydrogen couple (pH 6, 1 bar hydrogen equals Eº (H⁺/H₂)=−0.355V vs SHE).

By using a hydrogenase with a small overpotential, such as the [FeFe]hydrogenase CaHydA1, the compounds 1-nitrohexane and 1-nitropentane could be readily reduced in accordance with the disclosed methods (see example 9). Control experiments using oxidoreductase enzymes which have significant overpotentials (for example, E. coli Hyd1 has an overpotential for H₂ oxidation of around 80 mV) did not result in efficient reduction. It is straight forward to determine the overpotential at which a hydrogenase operates.¹

### Example 11

Reduction of further substrates was demonstrated using E. coli Hyd-1-modified carbon particles. Reactions were performed in a sodium phosphate buffer pH 6 at room temperature under hydrogen gas flow at atmospheric pressure. Reaction products were analysed using NMR. Substrates 8-13, 15-24, and 26-29 were run with 10% v/v MeCN as a co-solvent.

Reactions are shown in the table below. All reactions reached full conversion to the corresponding amine (i.e. 100% conversion) in the time indicated.

This example thus demonstrates the broad applicability of the methods disclosed herein. A wide range of substituents on the aromatic group carrying the reducible nitrogen-containing group (nitro group) are tolerated and full reduction of the nitro group was readily achieved under the experimental conditions.

TABLE 6

| Entry | Substrate | Time (h) |
|---|---|---|
| 1 | 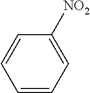 | 24 |
| 2 | 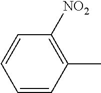 | 24 |
| 3 | 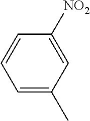 | 24 |
| 4 | 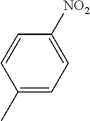 | 24 |
| 5 | 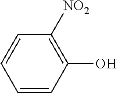 | 24 |
| 6 | 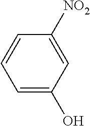 | 48 |
| 7 | 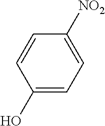 | 24 |
| 8 | 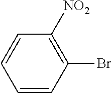 | 24* |
| 9 | 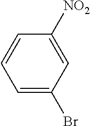 | 24 |
| 10 | 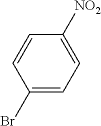 | 24 |
| 11 | 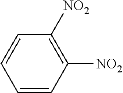 [SMILES: O=[N+]([O-])c1ccccc1[N+](=O)[O-]] | 72 |
| 12 | 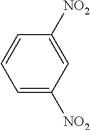 [SMILES: O=[N+]([O-])c1cccc([N+](=O)[O-])c1] | 48* |
| 13 | 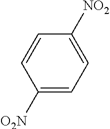 [SMILES: O=[N+]([O-])c1ccc([N+](=O)[O-])cc1] | 24 |
| 14 | 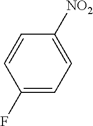 | 24* |
| 15 | 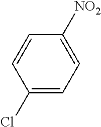 | 24 |
| 16 | 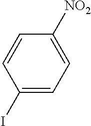 | 24 |
| 17 | 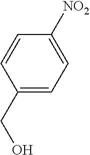 [SMILES: O=[N+]([O-])c1ccc(CO)cc1] | 48* |
| 18 | 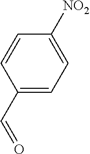 [SMILES: O=Cc1ccc([N+](=O)[O-])cc1] | 72* |
| 19 | 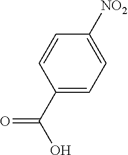 [SMILES: O=C(O)c1ccc([N+](=O)[O-])cc1] | 48* |
| 20 | 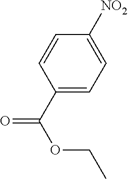 | 48* |
| 21 | 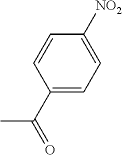 | 48 |
| 22 | 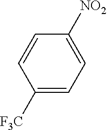 [SMILES: O=[N+]([O-])c1ccc(C(F)(F)F)cc1] | 72 |
| 23 | 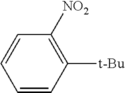 [SMILES: CC(C)(C)c1ccccc1[N+](=O)[O-]] | 72* |
| 24 | 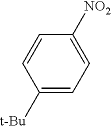 [SMILES: CC(C)(C)c1ccc([N+](=O)[O-])cc1] | 24* |
| 25 | 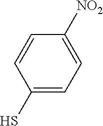 | 24 |
| 26 | 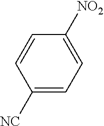 [SMILES: N#Cc1ccc([N+](=O)[O-])cc1] | 48* |
| 27 | 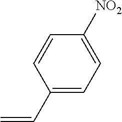 | 24* |
| 28 | 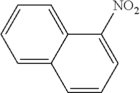 [SMILES: O=[N+]([O-])c1cccc2ccccc12] | 24 |
| 29 | 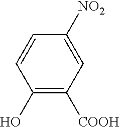 [SMILES: O=C(O)c1cc([N+](=O)[O-])ccc1O] | 48* |

*For substrates 8, 12, 14, 17-20, 23, 24, 26, and 29 amount of catalyst was doubled. For substrate 27 four times more catalyst was used.

### Example 12

Reduction of further substrates was demonstrated using E. coli Hyd-2-modified carbon particles. Reactions were performed in a sodium phosphate buffer pH 6 at room temperature under 2 bar hydrogen gas in a pressure vessel. Reaction products were analysed using NMR.

Substrates are shown in the table below. Both substrates were reduced efficiently and only the amine was detected as the reaction product, implying full conversion.

This example further demonstrates the broad applicability of the methods disclosed herein to aliphatic compounds as well as aromatic compounds.

TABLE 7

| Entry | Substrate |
|---|---|
| 1 | 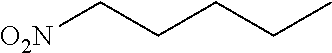 |
| 2 | 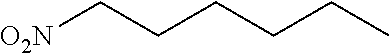 |

### REFERENCES

1 M. J. Lukey, A. Parkin, M. M. Roessler, B. J. Murphy, J. Harmer, T. Palmer, F. Sargent and F. A. Armstrong, J. Biol. Chem., 2010, 285, 3928-38.
2 X. Zhao, S. E. Cleary, C. Zor, N. Grobert, H. A. Reeve and K. A. Vincent, Chem. Sci., DOI:10.1039/D1SC00295C.
3 C. A. Marques, M. Selva and P. Tundo, J. Org. Chem., 1993, 58, 5256-5260.
4 US 2020/0262784 A1, 2020.
5 M. Rueping, C. Vila and U. Uria, Org. Lett., 2012, 14, 768-771.

SEQUENCE LISTING

SEQ ID NO: 1-Escherichia coli Hydrogenase 1 large subunit (hyaB)
MSTQYETQGYTINNAGRRLVVDPITRIEGHMRCEVNINDQNVITNAVSCGTMFRGLEIILQGRDPR
DAWAFVERICGVCTGVHALASVYAIEDAIGIKVPDNANIIRNIMLATLWCHDHLVHFYQLAGMDWI
DVLDALKADPRKTSELAQSLSSWPKSSPGYFFDVQNRLKKFVEGGQLGIFRNGYWGHPQYKLPPEA
NLMGFAHYLEALDFQREIVKIHAVFGGKNPHPNWIVGGMPCAINIDESGAVGAVNMERLNLVQSII
TRTADFINNVMIPDALAIGQFNKPWSEIGTGLSDKCVLSYGAFPDIANDFGEKSLLMPGGAVINGD
FNNVLPVDLVDPQQVQEFVDHAWYRYPNDQVGRHPFDGITDPWYNPGDVKGSDTNIQQLNEQERYS
WIKAPRWRGNAMEVGPLARTLIAYHKGDAATVESVDRMMSALNLPLSGIQSTLGRILCRAHEAQWA
AGKLQYFFDKLMTNLKNGNLATASTEKWEPATWPTECRGVGFTEAPRGALGHWAAIRDGKIDLYQC
VVPTTWNASPRDPKGQIGAYEAALMNTKMAIPEQPLEILRTLHSFDPCLACSTHVLGDDGSELISV
QVR
SEQ ID NO: 2-Escherichia coli Hydrogenase 1 small subunit (hyaA)
MNNEETFYQAMRRQGVTRRSFLKYCSLAATSLGLGAGMAPKIAWALENKPRIPVVWIHGLECTCCT
ESFIRSAHPLAKDVILSLISLDYDDTLMAAAGTQAEEVFEDIITQYNGKYILAVEGNPPLGEQGMF
CISSGRPFIEKLKRAAAGASAIIAWGTCASWGCVQAARPNPTQATPIDKVITDKPIIKVPGCPPIP
DVMSAIITYMVTFDRLPDVDRMGRPLMFYGQRIHDKCYRRAHFDAGEFVQSWDDDAARKGYCLYKM
GCKGPTTYNACSSTRWNDGVSFPIQSGHGCLGCAENGFWDRGSFYSRVVDIPQMGTHSTADTVGLT
ALGVVAAAVGVHAVASAVDQRRRHNQQPTETEHQPGNEDKQA
SEQ ID NO: 3-Escherichia coli hydrogenase 2 large subunit (hybC)
MSQRITIDPVTRIEGHLRIDCEIENGVVSKAWASGTMWRGMEEIVKNRDPRDAWMIVQRICGVCTT
THALSSVRAAESALNIDVPVNAQYIRNIILAAHTTHDHIVHFYQLSALDWVDITSALQADPTKASE
MLKGVSTWHLNSPEEFTKVQNKIKDLVASGQLGIFANGYWGHPAMKLPPEVNLIAVAHYLQALECQ
RDANRVVALLGGKTPHIQNLAVGGVANPINLDGLGVLNLERLMYIKSFIDKLSDFVEQVYKVDTAV
IAAFYPEWLTRGKGAVNYLSVPEFPTDSKNGSFLFPGGYIENADLSSYRPITSHSDEYLIKGIQES
AKHSWYKDEAPQAPWEGTTIPAYDGWSDDGKYSWVKSPTFYGKTVEVGPLANMLVKLAAGRESTQN
KLNEIVAIYQKLTGNTLEVAQLHSTLGRIIGRTVHCCELQDILQNQYSALITNIGKGDHTTFVKPN
IPATGEFKGVGFLEAPRGMLSHWMVIKDGIISNYQAVVPSTWNSGPRNFNDDVGPYEQSLVGTPVA
DPNKPLEVVRTIHSFDPCMACAVHVVDADGNEVVSVKVL
SEQ ID NO: 4-Escherichia coli hydrogenase 2 small subunit (hybO)
MTGDNTLIHSHGINRRDFMKLCAALAATMGLSSKAAAEMAESVTNPQRPPVIWIGAQECTGCTESL
LRATHPTVENLVLETISLEYHEVLSAAFGHQVEENKHNALEKYKGQYVLVVDGSIPLKDNGIYCMV
AGEPIVDHIRKAAEGAAAIIAIGSCSAWGGVAAAGVNPTGAVSLQEVLPGKTVINIPGCPPNPHNF
LATVAHIITYGKPPKLDDKNRPTFAYGRLIHEHCERRPHFDAGRFAKEFGDEGHREGWCLYHLGCK
GPETYGNCSTLQFCDVGGVWPVAIGHPCYGCNEEGIGFHKGIHQLANVENQTPRSQKPDVNAKEGG
NVSAGAIGLLGGVVGLVAGVSVMAVRELGRQQKKDNADSRGE
SEQ ID NO: 5-Ralstonia eutropha membrane-bound hydrogenase
moiety (HoxG)
MSAYATQGFNLDDRGRRIVVDPVTRIEGHMRCEVNVDANNVIRNAVSTGTMWRGLEVILKGRDPRD
AWAFVERICGVCTGCHALASVRAVENALDIRIPKNAHLIREIMAKTLQVHDHAVHFYHLHALDWVD
VMSALKADPKRTSELQQLVSPAHPLSSAGYFRDIQNRLKRFVESGQLGPFMNGYWGSKAYVLPPEA
NLMAVTHYLEALDLQKEWVKIHTIFGGKNPHPNYLVGGVPCAINLDGIGAASAPVNMERLSFVKAR
IDEIIEFNKNVYVPDVLAIGTLYKQAGWLYGGGLAATNVLDYGEYPNVAYNKSTDQLPGGAILNGN
WDEVFPVDPRDSQQVQEFVSHSWYKYADESVGLHPWDGVTEPNYVLGANTKGTRTRIEQIDESAKY
SWIKSPRWRGHAMEVGPLSRYILAYAHARSGNKYAERPKEQLEYSAQMINSAIPKALGLPETQYTL
KQLLPSTIGRTLARALESQYCGEMMHSDWHDLVANIRAGDTATANVDKWDPATWPLQAKGVGTVAA
PRGALGHWIRIKDGRIENYQCVVPTTWNGSPRDYKGQIGAFEASLMNTPMVNPEQPVEILRTLHSF
DPCLACSTHVMSAEGQELTTVKVR
SEQ ID NO: 6-Ralstonia eutropha membrane-bound hydrogenase
moiety (HoxK)
MVETFYEVMRRQGISRRSFLKYCSLTATSLGLGPSFLPQIAHAMETKPRTPVLWLHGLECTCCSES
FIRSAHPLAKDVVLSMISLDYDDTLMAAAGHQAEAILEEIMTKYKGNYILAVEGNPPLNQDGMSCI
IGGRPFIEQLKYVAKDAKAIISWGSCASWGCVQAAKPNPTQATPVHKVITDKPIIKVPGCPPIAEV
MTGVITYMLTFDRIPELDRQGRPKMFYSQRIHDKCYRRPHFDAGQFVEEWDDESARKGFCLYKMGC
KGPTTYNACSTTRWNEGTSFPIQSGHGCIGCSEDGFWDKGSFYDRLTGISQFGVEANADKIGGTAS
VVVGAAVTAHAAASAIKRASKKNETSGSEH
SEQ ID NO: 7-Ralstonia eutropha membrane-bound hydrogenase
moiety (HoxZ)
MSTKMQADRIADATGTDEGAVASGKSIKATYVYEAPVRLWHWVNALAIVVLAVTGFFIGSPPATRP
GEASANFLMGYIRFAHFVAAYIFAIGMLGRIYWATAGNHHSRELFSVPVFTRAYWQEVISMLRWYA
FLSARPSRYVGHNPLARFAMFFIFFLSSVFMILTGFAMYGEGAQMGSWQERMFGWVIPLLGQSQDV
HTWHHLGMWFIVVFVIVHVYAAIREDIMGRQSVVSTMVSGYRTFKD
SEQ ID NO: 8-Ralstonia eutropha regulatory hydrogenase moiety
(HoxB)
MNAPVCTGLASAKPGVLNVLWIQSGGCGGCSMSLLCADTTDFTGMLKSAGIHMLWHPSLSLESGVE
QLQILEDCLQGRVALHALCVEGAMLRGPHGTGRFHLLAGTGVPMIEWVSRLAAVADYTLAVGTCAA
YGGITAGGGNPTDACGLQYEGDQPGGLLGLNYRSRAGLPVINVAGCPTHPGWVTDALALLSARLLT
ASDLDTLGRPRFYADQLVHHGCTRNEYYEFKASAEKPSDLGCMMENMGCKGTQAHADCNTRLWNGE
GSCTRGGYACISCTEPGFEEPGHPFHQTPKVAGIPIGLPTDMPKAWFVALASLSKSATPKRVKLNA
TADHPLIAPAIRKTRLK
SEQ ID NO: 9-Ralstonia eutropha regulatory hydrogenase moiety
(HoxC)
MERLVVGPFNRVEGDLEVNLEVASGRVCSARVNATMYRGLEQILLHRHPLDALVYAPRVCGICSVS
QSVAASRALADLAGVTVPANGMLAMNLMLATENLADHLTHFYLFFMPDFTREIYAGRPWHTDATAR
FSPTHGKHHRLAIAARQRWFTLMGTLGGKWPHTESVQPGGSSRAIDAAERVRLLGRVREFRCFLEQ
TLYAAPLEDVVALDSEVALWRWHAQAPQAGDLRCFLTIAQDAALDQMGPGPGTYLSYGAYPQPEGG
FCFAQGVWRSAQGRLDALDLAAISEDATSAWLVDQGGARHPANGLTAPAPDKVGAYTWNKAPRLAG
AVLETGAIARQLAGAQPLVRDAVARCGATVYTRVLARLVELARVVPLMEDWLQSLEIGAPYWASAH
LPDQGAGVGLTEAARGSLGHWVSVRDGRIDNYQIVAPTSWNFSPRDIAGQPGAVEKALEGAPVLQG
ETTPVAVQHIVRSFDPCMVCTVH
SEQ ID NO: 10-Aquifex aeolicus hydrogenase large subunit (mbhL3)
MKIEKLVLTRVEGEASLNLVWERGVIKDAKISFYSTRGIEKVLRERPFMDALVINPRICGICGHAH
LIATVRAIENAIGIKEIPEKAKITRLVTQITEMVQNHVKWFYLFVMPDFLKFKESLSQFEPFKGER
WKRAVQFSSQIVKIIALFGGQWPHSSYAVPGGITSNFSEREVLKALNLVKEGKTFFEKNVSEDLEL
FLNLCEEFNLLSIGKAYNRFLSGGGLAYCENPSYKKGKGKVCKENVRYVQELEAADYSKANPVRYK
GLPYETGPLARELISKNPLVLKLYKNYGDSYAVRVAARLAEIKDLLEILEKLLKELMNHLEEPSCL
WEDRSDESGEGFGVVEAARGILIHRVVIEKGKIKDYKVITPSQWNLGPRCKKYLGVAEKAIVGLDS
ELKAQMVLRSFDLCSVCTTK
SEQ ID NO: 11-Aquifex aeolicus Hydrogenase small subunit (mbhS1)
METFWEVFKRHGVSRRDFLKFATTITGLMGLAPSMVPEVVRAMETKPRVPVLWIHGLECTCCSESF
IRSATPLASDVVLSMISLEYDDTLSAAAGEAVEKHRERIIKEYWGNYILAVEGNPPLGEDGMYCII
GGRPFVEILKESAEGAKAVIAWGSCASWGCVQAAKPNPTTAVPIDKVIKDKPIIKVPGCPPIAEVM
TGVIMYMVLFDRIPPLDSQGRPKMFYGNRIHDTCYRRSFFNAGQFVEQFDDEGAKKGWCLYKVGCR
GPTTYNSCGNMRWYNGLSYPIQSGHGCIGCAENNFWDNGPFYERIGGIPVPGIESKADKVGAIAAA
AAAGGAIIHGIASKIRKSGEKEE
SEQ ID NO: 12-Hydrogenovibrio marinus hydrogenase moiety (hoxK)
MSSQVETFYEVMRRQGITRRSFLKYCSLTAAALGLSPAYANKIAHAMETKPRTPVIWLHGLECTCC
SESFIRSAHPLAKDVVLSMISLDYDDTLMAASGHAAEAILDEIKEKYKGNYILAVEGNPPLNQDGM
SCIIGGRPFSEQLKRMADDAKAIISWGSCASWGCVQAAKPNPTQATPVHKFLGGGYDKPIIKVPGC
PPIAEVMTGVITYMLTFDRIPELDRQGRPKMFYSQRIHDKCYRRPHFDAGQFVEEWDDEGARKGYC
LYKVGCKGPTTYNACSTVRWNGGTSFPIQSGHGCIGCSEDGFWDKGSFYSRDTEMNAFGIEATADD
IGKTAIGVVGAAVVAHAAISAVKAAQKKGDK
SEQ ID NO: 13-Hydrogenovibrio marinus hydrogenase moiety (hoxG)
MSVLNTPNHYKMDNSGRRVVIDPVTRIEGHMRCEVNVDENNVIQNAVSTGTMWRGLEVILRGRDPR
DAWAFVERICGVCTGCHALASVRAVEDALDIKIPHNATLIREIMAKTLQIHDHIVHFYHLHALDWV
NPVNALKADPQATSELQKLVSPHHPMSSPGYFKDIQIRIQKFVDSGQLGIFKNGYWSNPAYKLSPE
ADLMAVTHYLEALDFQKEIVKIHAIFGGKNPHPNYMVGGVPCAINIDGDMAAGAPINMERLNFVKS
LIEQGRTFNTNVYVPDVIAIAAFYRDWLYGGGLSATNVMDYGAYPKTPYDKSTDQLPGGAIINGDW
GKIHPVDPRDPEQVQEFVTHSWYKYPDETKGLHPWDGITEPNYELGSKTKGSRTNIIEIDESAKYS
WIKSPRWRGHAVEVGPLARYILAYAQGVEYVKTQVHTSLNRFNAVCRLLDPNHKDITDLKAFLGST
IGRTLARALESEYCGDMMLDDENQLISNIKNGDSSTANTDKWDPSSWPEHAKGVGTVAAPRGALAH
WIVIEKGKIKNYQCVVPTTWNGSPRDPKGNIGAFEASLMGTPMERPDEPVEVLRTLHSFDPCLACS
THVMSEEGEEMATVKVR
SEQ ID NO: 14-Thiocapsa roseopersicina Hydrogenase large subunit
(hupL)
MSVTTANGFELDTAGRRLVVDPVTRIEGHLRCEVNLDENNVIRNAVSTGTMWRGLEVILRGRDPRD
AWAFTERICGVCTGTHALTSVRAVEDALGIPIPENANSIRNIMHVTLQAHDHLVHFYHLHALDWVD
VVSALGADPKATSALAQSISDWPKSSPGYFRDVQNRLKREVESGQLGPFMNGYWGSPAYKLPPEAN
LMAVTHYLEALDFQKEIVKIHTVYGGKNPHPNWLVGGMPCAINVDGTGAVGAINMERLNLVSSIID
QTIAFIDKVYIPDLIAIASFYKDWTYGGGLSSQAVMSYGDIPDHANDMSSKNLLLPRGAIINGNLN
EIHEIDLRNPEEIQEFVDHSWFSYKDETRGLHPWDGVTEPNFVLGPNAVGSRTRIEALDEQAKYSW
IKAPRWRGHAMEVGPLARYVIGYAKGIPEFKEPVDKVLTDLGQPLEAIFSTLGRTAARGLEASWAA
HKMRYFQDKLVANIRAGDTATANVDNWDPKTWPKEARGVGTTEAPRGALGHWIVIKDGKIDNYQAV
VPTTWNGSPRDPAGNIGAFEASLLNTPLAKADEPLEILRTLHSFDPCLACATHIMGPDGEELTRIK
VR
SEQ ID NO: 15-Thiocapsa roseopersicina Hydrogenase small subunit
(hupS)
MPTTETYYEVMRRQGITRRSFLKFCSLTATALGLSPTFAGKIAHAMETKPRIPVVWLHGLECTCCS
ESFIRSAHPLVSDVILSMISLDYTILIMAAAGHQAEAILEEVRHKHAGNYILAVEGNPPLNQDGMS
CIIGGRPFLEQLLEMADSCKAVISWGSCASWGCVQAARPNPTRATPVHEVIRDKPVIKVPGCPPIA
EVMTGVLTYILTFDRLPELDRQGRPLMFYGQRIHDKCYRRPHFDAGQFVESWDDEGARRGYCLYKV
GCKGPTTYNACSTIRWNGGVSFPIQSGHGCIGCSEDGFWDKGSFYQHVIDTHAFGIEANADRTGIA
VATRRGAAHRAHAAVSVVKRVQQKKEEDQS
SEQ ID NO: 16-Alteromonas macleodii Hydrogenase large subunit
(HyaB)
MENTASNNRLVVDPITRIEGHLRIEAEMDGNTIKQAFSSGTSVRGIELILQGRDPRDAWAFAQRIC
GVCTLVHGMASVRAVEDAIRKAWRSNAKLGVAIGKPSMTSMPKGPMQHGKKGHRQSRTSIGVLSEA
EMAIPQNAQLIRNIMIATQYVHDHVMHFYHLHALDWVDVVSALDADPTRTAALAGQLSDYPRSSPG
YFKDMKQKVKTLVESGQLGIFSNAYWGHPGYKLPPEVNLMALAHYLDALTWQREVVKVHTIFGGKN
PHPNFVVGGVPSPINLNASTGINTSRLVQLQDAITQMKSFVDQVYYPDIVAIAGYYKEWGTRGEGL
GNFLTYGDLPMTSMDDPDSFLFPRGAILGRDLSKVHDLDLDDPSEIQEFVSSSWYRYSGGNASGLH
PFNGQTTLEYTGPKPPYKHLNVGAEYSWLKSPRWKGHAMEVGPLARVLMMYAKKDAAAQDIVNRSL
SILDLETSALFSTLGRTLARAVETKIVVNQLQSWYDQLLDNIAKGDTDTENPLYFDPTNWPIKGQG
VGVMEAPRGALGHWLVMQNGKIENYQCVVPTTWNAGPRDPNSQAGAYEAALQDKHTLHDPDQPLEI
LRTLHSFDPCLACAVHVMDETGEERLRLKVR
SEQ ID NO: 17-Alteromonas macleodii Hydrogenase small subunit
(hoxK)
MALPTLNKQLQASGISRRTFLKFCATTASLLALPQSAVADLATALGNARRPSVIWLPFQECTGCTE
AILRSHAPTLESLIFDHISLDYQHTIMAAAGEQAEDARRAAMNAHKGQYLLLVDGSVPVGNPGYST
ISGMSNVDMLRESAKDAAGIIAIGTCASFGGIPKANPNPTGAVAVSDIITDKPIVNISGCPPLPIA
ITAVLVHYLTFKRFPDLDELQRPLAFFGESIHDRCYRRPFFEQRKFAKSFDDEGAKNGWCLFELGC
KGPETFNACATVKWNQGTSFPIESGHPCLGCSEPDFWDKSSFYQALGPWEWYKSKPGKGAQKHAGK
NS
SEQ ID NO: 18-Allochromatium vinosum Membrane-bound hydrogenase
large subunit (hydL)
MSERIVVDPITRIEGHLRIEAQMDGATIAQAYSSGTMVRGIETILKGRDPRDAWAFVQRICGVCTL
VHGIASVRAVEDALRIELPLNAQLIRNLMIGAQYIHDHVMHFYHLHALDWVDVVSALSADPRATSE
LAQSISAWPKSSPGYFADTQKRIKTFVESGQLGIFANGYWGHPAYRLPPEANLMAVAHYLEALAWQ
RDTAKFHAIFGGKNPHPNFVVGGVPSPIDLDSDSALNAKRLAEVRNLIQSMRTFVDQVYVPDTLAI
AGFYKDWGERGEGLGNFLCYGDLPTGASLDPATFLFPRGAILDRDLSTIHEVDLEATGEIQEFVNH
SWYEYSVGNDRGLHPYEGQTNLEYDRRGGVAPPYKQLDVSDGYSWLKAPRWKGRSVEVGPLARVLM
LYATGHDQARELVDSTLSRLDLPVDALYSTLGRTAARALESKILVDAMQGWYDGLIANVKSGDTKT
FNETLWEPSSWPSRAQGVGIMEAPRGALGHWIVIEDGRIANYQAVVPSTWNAGPRDGRGQAGAYEA
ALQDNHQLVDVKQPIEILRTIHSFDPCIACAVHLADPESGESFQVRVV
SEQ ID NO: 19-Allochromatium vinosum membrane-bound hydrogenase
small subunit (hydS)
PSVVWLSFQECTGCTESLTRAHAPTLEDLILDFISLDYHHTLQAASGEAAEAARLQAMDENRGQYL
VIVDGSIPGPDANPGFSTVAGHSNYSILMETVEHAAAVIAVGTCAAFGGLPQARPNPTGAMSVMDL
VRDKPVINVPGCPPIPMVITGVIAHYLVFGRLPEVDGYGRPLAFYGQSIHDRCYRRPFYDKGLFAE
SFDDEGAKQGWCLYRLGCKGPTTYNACATMKWNDGTSWPVEAGHPCLGCSEPQFWDAGGFYEPVSV
PLTLGPATLLGAGAAGAVVGGGLAALSRKKGRDAAATRQPVTVDELEQKL
SEQ ID NO: 20-Salmonella enterica serovar NiFe hydrogenase
(hybO)
MTGDNTLITSHGINRRDFMKLCAALAATMGLSSKAAAEMAESVSNPQRPPVIWIGAQECTGCTESL
LRATHPTVENLVLETISLEYHEVLSAAFGHQVEENKHNALEKYKGQYVLVVDGSIPLKDNGIYCMV
AGEPIVDHIRKAADGAAAIIAIGSCSAWGGVAAAGVNPTGAVSLQEVLPGKTVINIPGCPPNPHNF
LATVAHIITYGTPPKLDAKNRPTFAYGRLIHEHCERRPHFDAGRFAKEFGDEGHRQGWCLYHLGCK
GPETWGNCSTLQFCDVGGVWPVAIGHPCYGCNEEGIGFHKGIHQLAHVENQTPRSEKPDVNMKEGG
NISAGAVGLLGGVVGLVAGVSVMAVRELGRQQKKDNADSRGE
SEQ ID NO: 21-Salmonella typhimurium Hydrogenase (hyaB)
MSNQYQTQGYTVNDAGRRLIVDPITRIEGHMRCEVNIDEQNVITNAVSCGTMFRGLEIILQGRDPR
DAWAFVERICGVCTGVHALASVYAIEDAIGIQVPDNANIIRNIMLATLWCHDHLVHFYQLAGMDWI
DVLNALKADPRATSQLAQSLSAWPMSSPGYFFDVQNRLKKFVDGGQLGIFRNGYWGHPQYKLSPEA
NLMGFAHYLEALDFQREIVKIHTIFGGKNPHPNWIVGGMPCAINLDQSGAVGAINMERLNLVQSII
TRTADFINNVMVPDALAIGQFNKAWSQIGTGLSDKCVLSYGAFPDIANDFSQQSLLMPGGAVINGD
FKNVMPVDLADPQQIQEFVDHAWYRYPDDRLGRHPFDGITDPWYNPGDVKGSDTHIQQLNEQERYS
WIKAPRWRGHAMEVGPLARTLIAYHKGDAATIESVDRMMSALKLPLSGIQSTLGRILCRAHEAQWA
VGKLQYFFDRLMTNLKNGDLATANTEKWEPASWPQHCRGIGFTEAPRGALGHWASIRDQKIELYQC
VVPTTWNASPRDPKKQIGAYEAALMGTQMAIPDQPLEILRTLHSFDPCLACSTHVLGDDGSELIAV
QVR
SEQ ID NO: 22-Escherichia coli Ni/Fe-hydrogenase 1 B-type
cytochrome subunit (strain K12) (hyaC)
MQQKSDNVVSHYVFEAPVRIWHWLTVLCMAVLMVTGYFIGKPLPSVSGEATYLFYMGYIRLIHFSA
GMVFTVVLLMRIYWAFVGNRYSRELFIVPVWRKSWWQGVWYEIRWYLFLAKRPSADIGHNPIAQAA
MFGYFLMSVFMIITGFALYSEHSQYAIFAPFRYVVEFFYWTGGNSMDIHSWHRLGMWLIGAFVIGH
VYMALREDIMSDDTVISTMVNGYRSHKFGKISNKERS
SEQ ID NO: 23-Desulfovibrio vulgaris Miyazaki F Periplasmic
[NiFe] hydrogenase small subunit (hydA)
MKISIGLGKEGVEERLAERGVSRRDFLKFCTAIAVTMGMGPAFAPEVARALMGPRRPSVVYLHNAE
CTGCSESVLRAFEPYIDTLILDTLSLDYHETIMAAAGDAAEAALEQAVNSPHGFIAVVEGGIPTAA
NGIYGKVANHTMLDICSRILPKAQAVIAYGTCATFGGVQAAKPNPTGAKGVNDALKHLGVKAINIA
GCPPNPYNLVGTIVYYLKNKAAPELDSLNRPTMFFGQTVHEQCPRLPHFDAGEFAPSFESEEARKG
WCLYELGCKGPVTMNNCPKIKFNQTNWPVDAGHPCIGCSEPDFWDAMTPFYQN
SEQ ID NO: 24-Desulfovibrio vulgaris Miyazaki F Periplasmic
[NiFe] hydrogenase large subunit (hydB)
MSGCRAQNAPGGIPVTPKSSYSGPIVVDPVTRIEGHLRIEVEVENGKVKNAYSSSTLERGLEIILK
GRDPRDAQHFTQRTCGVCTYTHALASTRCVDNAVGVHIPKNATYIRNLVLGAQYLHDHIVHFYHLH
ALDFVDVTAALKADPAKAAKVASSISPRKTTAADLKAVQDKLKTFVESGQLGPFTNAYFLGGHPAY
YLDPETNLIATAHYLEALRLQVKAARAMAVFGAKNPHTQFTVVGGVTCYDALTPQRIAEFEALWKE
TKAFVDEVYIPDLLVVAAAYKDWTQYGGTDNFITFGEFPKDEYDLNSRFFKPGVVFKRDFKNIKPF
DKMQIEEHVRHSWYEGAEARHPWKGQTQPKYTDLHGDDRYSWMKAPRYMGEPMETGPLAQVLIAYS
QGHPKVKAVTDAVLAKLGVGPEALFSTLGRTAARGIETAVIAEYVGVMLQEYKDNIAKGDNVICAP
WEMPKQAEGVGFVNAPRGGLSHWIRIEDGKIGNFQLVVPSTWTLGPRCDKNKLSPVEASLIGTPVA
DAKRPVEILRTVHSFDPCIACGVHVIDGHTNEVHKFRIL
SEQ ID NO: 25-Clostridium beijerinckii SM10 (CbA5H) [FeFe]-
hydrogenase (KX147468)
MGDNKKSFIQSALGSVFSVFSEEELKELSNGRKIAICGKVNNPGIIEVPEGATLNEIIQLCGGLIN
KSNFKAAQIGLPFGGFLTEDSLDKEFDFGIFYENIARTIIVLSQEDCIIQFEKFYIEYLLAKIKDG
SYKNYEVVKEDITEMFNILNRISKGVSNMREIYLLRNLAVTVKSKMNQKHNIMEEIIDKFYEEIEE
HIEEKKCYTSQCNHLVKLTITKKCIGCGACKRACPVDCINGELKKKHEIDYNRCTHCGACVSACPV
DAISAGDNTMLFLRDLATPNKVVITQMAPAVRVAIGEAFGFEPGENVEKKIAAGLRKLGVDYVEDT
SWGADLTIMEEAAELQERLERHLAGDESVKLPILISCCPSWIKFIEQNYGDMLDVPSSAKSPMEME
AIVAKEIWAKEKGLSRDEVTSVAIMPCIAKKYEASRAEFSVDMNYDVDYVITTRELIKIFENSGIN
LKEIEDEEIDTVMGEYTGAGIIFGRIGGVIEAATRTALEKMTGERFDNIEFEGLRGWDGFRVCELE
AGDIKLRIGVAHGLREAAKMLDKIRSGEEFFHAIEIMACVGGCIGGGGQPKTKGNKQAALQKRAEG
LNNIDRSKTLRRSNENPEVLAIYEKYLDHPLSNKAHELLHTVYFPRVKKD
SEQ ID NO: 26-Clostridium beijerinckii ATCC 51743 [FeFe]-
Hydrogenase (Cbei_4110)
MSKYMVIDGNRIEFDKEKNILDLVRKAGIDLPTLCYYTDLSVYGACRMCVVEDERGSILTSCSTPP
KDMMSIRTNTPKLQKYRKVILELLLATHCRDCTICEKNGKCKLQKLASRFGLTNIRFKSIEGKKDL
DTSSKSIIRDPNKCILCGDCVRMCNEIQSVGAIDFANRGSNMVVSPAFGKSLAETDCVNCGQCATV
CPTGAIVVKSDIKNVWKAIYNHKQRVIAQVAPAVRVALGEEFGIKPGENVMGKIVAAMRKLGFENI
YDTSLSADLTVIEESKEFLKKLESDDNKFPLFTSCCPAWVRYVENKYTELLPYVSSCKSPMEMFGS
VVKAYFKEKDSLENRETISVAVMPCTAKKAEAAREEFIRDNIPDVDYVITTAELCAMIKEIGIQFD
EIEAEASDIPLSLYSGAGVIFGVTGGVTEAVIREVVKDKSSRVLKDIEFIGVRGMKGVKTCELQVK
DESIRIGIVSGLRNAEDLIEKIKSGEEHFDFIEVMACPGGCIAGAGQPFGLMEEKNERAKGLYKID
KVTQIKRSEENLVVKSLYEGLLKNRTKELLHVHYDKSEH
SEQ ID NO: 27-Clostridium beijerinckii [FeFe]-Hydrogenase
(Cbei_1773)
MGDNKKSFIQSALGSVFSVFSEEELKELSNGRKIAICGKVNNPGIIEVPEGATLNEIIQLCGGLIN
KSNFKAAQIGLPFGGFLTEDSLDKEFDFGLFYENIARTIIVLSQEDCIIQFEKFYIEYLLAKIKDG
SYKNYEVVKEDITEMFNILNRISKGVSNMREIYLLRNLAVTVKSKMNQKHNIMEEIIDKFYEEIEE
HIEEKKCYTSQCNHLVKLTITKKCIGCGACKRACPVDCINGELKKKHEIDYNRCTHCGACVSACPV
DAISAGDNTMLFLRDLATPNKVVITQMAPAVRVAIGEAFGFEPGENVEKKIAAGLRKLGVDYVEDT
SWGADLTIMEEAAELQERLERHLAGDESVKLPILISCCPSWIKFIEQNYGDMLDVPSSAKSPMEME
AIVAKEIWAKEKGLSRDEVTSVAIMPCIAKKYEASRAEFSVDMNYDVDYVITTRELIKIFENSGIN
LKEIEDEEIDTVMGEYTGAGIIFGRTGGVIEAATRTALEKMTGERFDNIEFEGLRGWDGFRVCELE
AGDIKLRIGVAHGLREAAKMLDKIRSGEEFFHAIEIMACVGGCIGGGGQPKTKGNKQAALQKRAEG
LNNIDRSKILRRSNENPEVLAIYEKYLDHPLSNKAHELLHTVYFPRVKKD
SEQ ID NO: 28-Clostridium beijerinckii [FeFe]-Hydrogenase
(Cbei_3796)
MDIGFVNIDKELCTGCQQCVEVCPVNAIQGKKGQPQNIDYDVCVSCGQCIQVCNSYGFENRENSHL
IEEKRRDRGVLESVKEPVFAAFNKGNAAKVKEALHDEELFTIVQCAPAVRVSLGEEFGLKAGSLTA
GKMAAALRRLGFNRVYDTNFGADLTIMEEGSELIKRVTEGGELPMFTSCCPAWVKFMEQSYPELLN
HLSSCKSPQQMAGTIFKTYGAKIDKVNPKKIYNVAIMPCTCKQFECDREEMQDSGFKDVDIVITTR
EFAQLIRDNEIDFKNLKDEEFDLPLGSYTGAGNIFGVTGGVMEAALRSGYEMLTKKSIPNLELNFV
RGSEGIRVAEVKLPKITLKVAVVSGLKNVVQILEDIKEGKCDFDFIEVMTCPEGCVSGGGQPKFIL
DIDRRNALVSRKKGIYKHDSELEIRKSHENPFIKKLYEEFLIEPLGEKSHHLLHTKFVSRKKEEI
SEQ ID NO: 29-Clostridium pasteurianum [FeFe]-Hydrogenase (hydA)
MKSEYNDLFKSLIDAYYKDDFDEFIKKALSDSTVNKEELSNIISSFCGVELKYTDKDTYIKDLKNA
IKNYNSDHKIVTKIRDCSVDCADENGKTSCQKSCPFDAILIDEANKTSYIDKDLCTDCGFCVEGCP
NGSILDKVEFIPLANLLKEKQPVIAAVAPAITGQFGDDVTIDQLRTAFKKVGFADMIEVAFFADML
TLKEACEFNAHVKSKDDLMITSCCCPMWVGMLKRVYKDMVKYVSPSVSPMIAAGRVIKTLNSNCKV
VFVGPCIAKKAESKNKDIEGDIDFVLTFEEVKNIFESLNINPSELPEDPSTDYASREGRLYARTGG
VSISVSEAVAKLFPEKKDLFKSVQANGVIECKKILEKAQNGEVAANFIEGMGCVGGCVGGPKALIP
KEKGREKVNEFAENSNVKISLESDQMKKILNMLNITSAKDEMDEEKIKIFEREF
SEQ ID NO: 30-Chlamydomonas reinhardtii [FeFe]-hydrogenase
(hyd1)
MSALVLKPCAAVSIRGSSCRARQVAPRAPLAASTVRVALATLEAPARRLGNVACAAAAPAAEAPLS
HVQQALAELAKPKDDPTRKHVCVQVAPAVRVAIAETLGLAPGATTPKQLAEGLRRLGFDEVEDTLF
GADLTIMEEGSELLHRLTEHLEAHPHSDEPLPMFTSCCPGWIAMLEKSYPDLIPYVSSCKSPQMML
AAMVKSYLAEKKGIAPKDMVMVSIMPCTRKQSEADRDWFCVDADPTLRQLDHVITTVELGNIFKER
GINLAELPEGEWDNPMGVGSGAGVLFGTTGGVMEAALRTAYELFTGTPLPRLSLSEVRGMDGIKET
NITMVPAPGSKFEELLKHRAAARAEAAAHGTPGPLAWDGGAGFTSEDGRGGITLRVAVANGLGNAK
KLITKMQAGEAKYDFVEIMACPAGCVGGGGQPRSTDKAITQKRQAALYNLDEKSTLRRSHENPSIR
ELYDTYLGEPLGHKAHELLHTHYVAGGVEEKDEKK
SEQ ID NO: 31-Chlorella variabilis [FeFe]-hydrogenase (HYDA1)
MEALVRRGLQSPDQALRLLGVARAICVGLSRSLPALAASAEQFAEQYPKLKLKVNGREVTVPEGTS
VLNACREAGAYVPTLCTHPRLPTTPGTCRICMVETGGGQLKPACATPAWEGMEVQTATDKVQESIR
GVLSLMKANHPSDCMNCDASGRCEFQDLISRYNVKDVLPKLKTYSHEWDAEVQADFEHFHDSSSTA
LTLDLEKCIKCGRCVTMCGQVQQMNVLGMINRSRMAHPGVLIEEALDHSKCIECGQCSSVCPVGAI
VEHSEWRQVLDALENKQKVMVVQTAPSVRVSIGEELGLAPGTVETGQMVAAQRALGFDYVEDSDES
ADLTIMEEGTELLQRLGAAWRAETAAQDAAAGSWAAAKQGHGEGEAHGHAPGPLPMFTSCCPAWIN
LVEKSYPELIPHLSSCKSPQMMMGAVVKHYWAKKKGLKPEDVCLVGIMPCTAKKHETERKEFRNEN
GAYDCDYVITTREFGHMLRHKKIPMPSLKPEEFDNPLGEATGAAALFGATGGVMEAAIRTAYEIAA
GEPLPKLEVEAVRGVKGVKEATLTLPANDTTLKAGVAGKEIRVAVASGIGNARHLLQRIQAGEAHY
DFVEVMACPGGCIGGGGQPKTHDPDAVLKRMGAIYQVDKSLALRKSHENPSIHKIYAEFLGQPGGE
LSHKLLHTHYTDHSVDTLPSVRELGGSGEVAKRAALTAAGEMRYKRIAMVGDPSAKR
SEQ ID NO: 32-Chlorella variabilis [Fe-Fe] hydrogenase (HYD2)
MATIHINGHTVSVPEGTSILTAATQLGIHIPTLCTHPRLPTTPGTCRLCLVEVAGGALKPACATPV
CHGLEVTTDSPQVKDSIRGVLALLKANHPADCMTCDVSGRCEFQARPGFWGGAWAGQRMACPRQRP
MLPMLGCRCLKCGRCVTACGLVQEMDVLGMKGRSRERHPAVLTEAMDLSKCISCGQCAVMCPVGAI
TERAEWREVEDQLDAKRKAGRGAGRAGLMVCVTAPAVRVAIGEELGLAPGTITTGQMVAAQRQLGF
DYVFDVNFGADLTIMEEGTELLQRLRHAWGLDLPAEGSGGAGAGPLPMFTSCCPGWVTACEKSFPE
LLPHLSTCKSPQQMMGAVVKSHFAAKLGKRAQDICLVSVMPCTAKKYEAERGEMVREGEGPDVDYV
ITTREFGRLLRERHIPLASLPESAFDNPLGEGSGAGVIFGNTGGVMEAALRTAYELAAGQPLPKLE
EEAIRGLRGIKQATVTLPPTAPAGMASRQLRVAVASGIGQARHLLERMHTGHSPHFDFVEVMACPG
GCVGGGGQPKSADPLVLLKRMGAVYSIDERSAIRKSHENPSIQKLYKACAEFLGEPGGSLSHQLLH
TTYINRSTASQPTYTAFQRMDEPCNPKLQQAAAAGRGSSSSALPGTSA
SEQ ID NO: 33-Ralstonia eutropha soluble hydrogenase moiety
(HoxH)
MSRKLVIDPVTRIEGHGKVVVHLDDDNKVVDAKLHVVEFRGFEKFVQGHPFWEAPMFLQRICGICF
VSHHLCGAKALDDMVGVGLKSGIHVTPTAEKMRRLGHYAQMLQSHTTAYFYLIVPEMLFGMDAPPA
QRNVLGLIEANPDLVKRVVMLRKWGQEVIKAVFGKKMHGINSVPGGVNNNLSIAERDRELNGEEGL
LSVDQVIDYAQDGLRLFYDFHQKHRAQVDSFADVPALSMCLVGDDDNVDYYHGRLRIIDDDKHIVR
EFDYHDYLDHFSEAVEEWSYMKFPYLKELGREQGSVRVGPLGRMNVTKSLPTPLAQEALERFHAYT
KGRTNNMTLHTNWARAIEILHAAEVVKELLHDPDLQKDQLVLTPPPNAWTGEGVGVVEAPRGTLLH
HYRADERGNITFANLVVATTQNNQVMNRTVRSVAEDYLGGHGEITEGMMNAIEVGIRAYDPCLSCA
THALGQMPLVVSVEDAAGRLIDERAR
SEQ ID NO: 34-Ralstonia eutropha soluble hydrogenase moiety
(HOXY)
MRAPHKDEIASHELPATPMDPALAANREGKIKVATIGLCGCWGCTLSFLDMDERLLPLLEKVTLLR
SSLIDIKRIPERCAIGFVEGGVSSEENIETLEHFRENCDILISVGACAVWGGVPAMRNVFELKDCL
AEAYVNSATAVPGAKAVVPFHPDIPRITTKVYPCHEVVKMDYFIPGCPPDGDAIFKVLDDLVNGRP
FDLPSSINRYD
SEQ ID NO: 35-Rhodococcus opacus SH hydrogenase moiety (HoxH)
MSTKLVIDPVTRIEGHGKVTVHLDDNNNVVDAHLHVVEFRGFEKLVQGHPFWEAPMLMQRICGICF
VSHHLCGAKALDDMVGVGLKSGIDVTPTAEKIRRLGHYAQMLQSHATAYFYLIVPEMLFGMDAAPE
QRNVLGLIEANPELVKRVVMLRKWGQEVIKAVFGRRMHGISSVPGGVNKNLSVAECQRFLKGEEGL
PSVDEVIEYAQEGVQLFYDFHEQNRVQVDSFANVSALSMSLVDADGNVDYYHGKLRIIDDDKNVVQ
EFDYHDYLDHFSEAVEEWSYMKFPFLKALGRERGSVRVGPLGRLNVINSLSTPLAQEALERFHAYT
NGKANNMTLHTNWARAIEILHAAELIKELLNDPDLQKEQLLLTPADNAWTGEGVGVVEAPRGTLLH
HYRADQEGDITFANLVVATTQNNQVMNRTVRSVAEDYLGGQGEVTEGMMNAIEVGIRAYDPCLSCA
THALGQMPLIVSVHDTEGHVINERVR
SEQ ID NO: 36-Rhodococcus opacus SH hydrogenase moiety (HoxY)
MKHSEKNEIASHELPTTPLDPVLAAGRESKIKVAMIGLCGCWGCTLSFLDMDERLLVLLDKVTLHR
SSLSDIKRITERCAIGFIEGGVANEENIETLEHYRENCDVLISVGACAVWGGVPAMRNVFELKDCL
SEVYIDSATSVPGAKPVVPFHPDIPRITDKVYPCHEVVKMDYFIPGCPPDADAIFKVLDDLVNGRP
FDLPSSINQYD
SEQ ID NO: 37-Desulfovibrio fructosovorans nickel-iron
hydrogenase large subunit.
MAESKPTPQSTFTGPIVVDPITRIEGHLRIMVEVENGKVKDAWSSSQLFRGLEIILKGRDPRDAQH
FTQRACGVCTYVHALASSRCVDDAVKVSIPANARMMRNLVMASQYLHDHLVHFYHLHALDWVDVTA
ALKADPNKAAKLAASIDTARTGNSEKALKAVQDKLKAFVESGQLGIFTNAYFLGGHKAYYLPPEVN
LIATAHYLEALHMQVKAASAMAILGGKNPHTQFTVVGGCSNYQGLTKDPLANYLALSKEVCQFVNE
CYIPDLLAVAGFYKDWGGIGGTSNYLAFGEFATDDSSPEKHLATSQFPSGVITGRDLGKVDNVDLG
AIYEDVKYSWYAPGGDGKHPYDGVTDPKYTKLDDKDHYSWMKAPRYKGKAMEVGPLARTFIAYAKG
QPDFKKVVDMVLGKLSVPATALHSTLGRTAARGIETAIVCANMEKWIKEMADSGAKDNTLCAKWEM
PEESKGVGLADAPRGSLSHWIRIKGKKIDNFQLVVPSTWNLGPRGPQGDKSPVEEALIGTPIADPK
RPVEILRTVHAFDPCIACGVHVIEPETNEILKFKVC
SEQ ID NO: 38-Desulfovibrio fructosovorans nickel-iron
hydrogenase small subunit.
LTAKHRPSVVWLHNAECTGCTEAAIRTIKPYIDALILDTISLDYQETIMAAAGETSEAALHEALEG
KDGYYLVVEGGLPTIDGGQWGMVAGHPMIETCKKAAAKAKGIICIGTCSPYGGVQKAKPNPSQAKG
VSEALGVKTINIPGCPPNPINFVGAVVHVLIKGIPDLDENGRPKLFYGELVHDNCPRLPHFEASEF
APSFDSEEAKKGFCLYELGCKGPVTYNNCPKVLFNQVNWPVQAGHPCLGCSEPDFWDTMTPFYEQG
SEQ ID NO: 39-Clostridium acetobutylicum iron-iron hydrogenase.
MKTIILNGNEVHTDKDITILELARENNVDIPTLCFLKDCGNFGKCGVCMVEVEGKGFRAACVAKVE
DGMVINTESDEVKERIKKRVSMLLDKHEFKCGQCSRRENCEFLKLVIKTKAKASKPFLPEDKDALV
DNRSKAIVIDRSKCVLCGRCVAACKQHTSTCSIQFIKKDGQRAVGTVDDVCLDDSTCLLCGQCVIA
CPVAALKEKSHIEKVQEALNDPKKHVIVAMAPSVRTAMGELFKMGYGKDVTGKLYTALRMLGFDKV
FDINFGADMTIMEEATELLGRVKNNGPFPMFTSCCPAWVRLAQNYHPELLDNLSSAKSPQQIFGTA
SKTYYPSISGIAPEDVYTVTIMPCNDKKYEADIPFMETNSLRDIDASLTTRELAKMIKDAKIKFAD
LEDGEVDPAMGTYSGAGAIFGATGGVMEAAIRSAKDFAENKELENVDYTEVRGFKGIKEAEVEIAG
NKLNVAVINGASNFFEFMKSGKMNEKQYHFIEVMACPGGCINGGGQPHVNALDRENVDYRKLRASV
LYNQDKNVLSKRKSHDNPAIIKMYDSYFGKPGEGLAHKLLHVKYTKDKNVSKHE
SEQ ID NO: 40-Desulfomicrobium baculatum nickel-iron-selenium
hydrogenase large subunit.
MSQAATPAADGKVKISIDPLTRVEGHLKIEVEVKDGKVVDAKCSGGMFRGFEQILRGRDPRDSSQI
VQRICGVCPTAHCTASVMAQDDAFGVKVTINGRITRNLIFGANYLQSHILHFYHLAALDYVKGPDV
SPFVPRYANADLLTDRIKDGAKADATNTYGLNQYLKALEIRRICHEMVAMFGGRMPHVQGMVVGGA
TEIPTADKVAEYAARFKEVQKFVIEEYLPLIYTLGSVYTDLFETGIGWKNVIAFGVFPEDDDYKTF
LLKPGVYIDGKDEEFDSKLVKEYVGHSFFDHSAPGGLHYSVGETNPNPDKPGAYSFVKAPRYKDKP
CEVGPLARMWVQNPELSPVGQKLLKELYGIEAKNFRDLGDKAFSIMGRHVARAEETWLTAVAVEKW
LKQVQPGAETYVKSEIPDAAEGTGFTEAPRGALLHYLKIKDKKIENYQIVSATLWNANPRDDMGQR
GPIEEALIGVPVPDIKNPVNVGRLVRSYDPALGCAVH
SEQ ID NO: 41-Desulfomicrobium baculatum nickel-iron-selenium
hydrogenase small subunit.
MTEGAKKAPVIWVQGQGCTGCSVSLLNAVHPRIKEILLDVISLEFHPTVMASEGEMALAHMYEIAE
KFNGNFFLLVEGAIPTAKEGRYCIVGETLDAKGHHHEVTMMELIRDLAPKSLATVAVGTCSAYGGI
PAAEGNVTGSKSVRDFFADEKIEKLLVNVPGCPPHPDWMVGTLVAAWSHVLNPTEHPLPELDDDGR
PLLFFGDNIHENCPYLDKYDNSEFAETFTKPGCKAELGCKGPSTYADCAKRRWNNGINWCVENAVC
IGCVEPDFPDGKSPFYVAE
SEQ ID NO: 42-Hydrogenophilus thermoluteolus SH moiety HoxH.
MTQHAPQAVSPRPSLPANATRRVAIDPLSRVEGHGKVTIWLDDDGQVVEARLHIVEFRGFEAFIVG
RPYWEAPVVVQRLCGICPVSHHLAAAKALDRLVGVTQLPPTAEKMRRLMHYGQVLQSHALHFFYLA
APDLLLGFSADPAQRNVFGLAAQKRELARQGILVRQFGQECIEATAGKRIHGTSAVPGGIHKNLSR
RERMALLSRAPEIRSWCEAAVALIERLFTEHAPFFAQFGSFQTKTFSLVAADGSLDLYDGTFRVKE
ANGAILIDHYDPNDYDQLLVEAVRPWSYMKFPYLKAYGEPDGFYRVGPSARLINCDRLTTARAEAA
RQRFLTFDQGTVAHSTLGYHWARLIEMLHCAELIEALLTDADLEGGELRARGQRQHRGVGVIEAPR
GTLIHHYEVGDDDLITYCNLIVSTTHNNAVMNQAVTTAAKAFLSGVTLTEALLNHIEVAVRAFDPC
LSCATH
SEQ ID NO: 43-Hydrogenophilus thermoluteolus SH moiety HoxY.
MTSAAPSAMPPRKIRIATASLAGCFGCHMSFADIDTRLLALAEWVTFDRSPLTDWKTVGECDIALI
EGGVCNAENVEVLRAYRRAARILVAVGACAINGGLPAQRNQHRVERLLTQVFEADRHLAPGSRVPN
DPELPLLLEHVHPIHEIVRVDYYLPGCPPTAEVIWTFLIDLLVGREPHFPYPTLRYD
SEQ ID NO: 44-Desulfovibrio gigas Periplasmic [NiFe] hydrogenase
Small subunit.
LTAKKRPSVVYLHNAECTGCSESVLRTVDPYVDELILDVISMDYHETLMAGAGHAVEEALHEAIKG
DFVCVIEGGIPMGDGGYWGKVGGRNMYDICAEVAPKAKAVIAIGTCATYGGVQAAKPNPTGTVGVN
EALGKLGVKAINIAGCPPNPMNFVGTVVHLLTKGMPELDKQGRPVMFFGETVHDNCPRLKHFEAGE
FATSFGSPEAKKGYCLYELGCKGPDTYNNCPKQLFNQVNWPVQAGHPCIACSEPNFWDLYSPFYSA
SEQ ID NO: 45-Desulfovibrio gigas Periplasmic [NiFe] hydrogenase
Large subunit.
MSEMQGNKIVVDPITRIEGHLRIEVEVEGGKIKNAWSMSTLFRGLEMILKGRDPRDAQHFTQRACG
VCTYVHALASVRAVDNCVGVKIPENATLMRNLTMGAQYMHDHLVHFYHLHALDWVNVANALNADPA
KAARLANDLSPRKTTTESLKAVQAKVKALVESGQLGIFTNAYFLGGHPAYVLPAEVDLIATAHYLE
ALRVQVKAARAMAIFGAKNPHTQFTVVGGCTNYDSLRPERIAEFRKLYKEVREFIEQVYITDLLAV
AGFYKNWAGIGKTSNFLTCGEFPTDEYDLNSRYTPQGVIWGNDLSKVDDENPDLIEEHVKYSWYEG
ADAHHPYKGVTKPKWTEFHGEDRYSWMKAPRYKGEAFEVGPLASVLVAYAKKHEPTVKAVDLVLKT
LGVGPEALFSTLGRTAARGIQCLTAAQEVEVWLDKLEANVKAGKDDLYTDWQYPTESQGVGFVNAP
RGMLSHWIVQRGGKIENFQLVVPSTWNLGPRCAEGKLSAVEQALIGTPIADPKRPVEILRTVHSYD
PCIACGVH
SEQ ID NO: 46-Pyrococcus furiosus soluble hydrogenase alpha
subunit.
MIIELDEFTRVEGNGKAEIVIENGEVKDARVKIVEGPRFFEILTLGRDYWDVPDLEARICAICYIA
HSVASVRAIEKALGIDVPESVEKLRELALWGEIIESHALHLYLLALPDVEGYPDAISMIPRHGELV
KEGLTIKAFGNAIRELIGGREIHGINIKPGGFGRYPSEEELEKIAEHSKSLIKFARRIVGIFASQE
AGGAVGEVLMATSDYLWGDELIINGERVQYYEVDEVPVGYSFAKHSYYKGNPVFVGALPRLLLKGE
SIEGEAARMLEEYRDKLESKYVIYNNLAQAIELLYALERVPQLVEEILSEGIERGNGEISQESGEG
VGYVEAPRGVLVHHYRIENGKVVWSNTITPTAFNQRLMELSLLEEAKRLYGSESEENMKKRLEVIV
RAFDPCISCSVHFVKL
SEQ ID NO: 47-Desulfovibrio vulgaris Hildenborough Carbon
monoxide dehydrogenase (cooS)
MSSSKTIRSRSIWDDAHAMLEKAKAEGISTVWDRAAEQTPACKFCELGTTCRNCIMGPCRIANRKD
GKMRLGVCGADADVIVARNFGRFIAGGAAGHSDHGRDLIETLEAVAEGKAPGYTIRDVAKLRRIAA
ELGVADAATRPAHDVAADLVTICYNDFGSRRNALAFLARAPQVRRDLWQRLGMTPRGVDREIAEMM
HRTHMGCDNDHTSLLVHAARTALADGWGGSMIGTELSDILFGTPRPRQSTVNLGVLRKDAVNILVH
GHNPVVSEMILAATREPAVRQAAQDAGAADINVAGLCCTGNELLMRQGIPMAGNHLMTELAIVTGA
ADAIVADYQCIMPSLVQIAACYHTRFVTTSPKGRFTGATHVEVHPHNAQERCREIVMLAIDAYTRR
DPARVDIPSQPVSIMSGFSNEAILEALGGTPKPLIDAVVAGQIRGFVGIVGCNNPKIRQDSANVTL
TRELIRRDIMVLATGCVTTAAGKAGLLVPEAASKAGEGLAAVCRSLGVPPVLHMGSCVDNSRILQL
CALLATTLGVDISDLPVGASSPEWYSEKAAAIAMYAVASGIPTHLGLPPNILGSENVTAMALHGLQ
DVVGAAFMVEPDPVKAADMLEAHIVARRARLGLTS
SEQ ID NO: 48-Desulfovibrio vulgaris Miyazaki Carbon monoxide
dehydrogenase (DvME)
MNSGTPDTERTLWEDARQMLRKAEAENIPTAWDRMREQTPHCKFCELGTTCRNCVMGPCRIANRKD
GKMRYGVCGADADVIVARNFGRFIAGGAAGHSDHGRDLIETLEAVVEGHAPGYTIRDEAKLRRIAG
ELGVADADTRPTLDVARDLVDVCYADFGSRRTELAFLKRAPKARRELWARLGMTPRGIDREIAEMM
HRTHMGCDNDPASLLTHAARTALADGWGGSMIGTELSDILFGTPMPRQSTVNLGVLREDAVNILVH
GHNPVVSEMILAAAREPAMQQAARDAGAAALNVAGLCCTGNELLMRQGIPMAGNHLMTELAIVTGA
ADAIVADYQCIMPSLVRIAACYHTRFVTTSSKGRFTGATHIEVHPHNAQEKCREIVQLAIEAFTRR
DPARVSIPVQPVPITTGFSNEAILAALGGTPAPLLDAVKAGQIRGFVGIVGCNNPKIMQDSGNVGL
AKELIRRDIMVLATGCVTTAAGKAGLLMPEAADMAGPGLSAVCKALGVPPVLHMGSCVDNSRILQL
CGLLADELGVDISDLPVGASSPEWYSEKAAAIGLYAVASGIPTHLGLPPNILGSDVVTGMAVDGLN
GLVGAAFMVEADPVKAADLLEAHIVARRQKLGLSA
SEQ ID NO: 49-Desulfovibrio psychrotolerans Carbon monoxide
dehydrogenase (cooS)
MTREQRSVEELSIWEDARSMLRKARAEGVETAWDRLEQQTPHCTFCELGTTCRNCVMGPCRISPKA
TKSGKLQRGVCGADADVIVARNFGRFIAGGSAGHSDHGRDLIEVLEAIVEGETTDYAIRDEAKLRR
IAAELGIDAAERPLMDVARDLVDECYSDFGSRRKSVGFLARAPEKRRALWEKLGMTPRGVDREIAE
MMHRTHMGCDNDAPNTLIHAARCALADGWSGSMIGTELSDIIFGVPTPSMSTANLGILKKDTVNIL
VHGHNPVVSEMILAAAREPQLIAAAKAHGASGITVGGLCCTGNELLMRQGIPMAGNHLMTELAIVT
GAVEAVVVDYQCIMPSLVQIAGCYHTRFITTAGKARFTGAIHFDIHPHNAMEEARKIVQMAVDAFA
ERDPKRVEIPVEPVRIMTGFSNEAILNALGGSLTPLLDAVKAGSIRGFVAIVGCNNPKIQQDSANV
GLAKALIERDIMVLATGCVTTAAGKAGLLVPEAASMAGPGLKAVCTALGIPPVLHMGSCVDNSRIM
HLCGLIANELGVDISDLPVGASSPEWYSEKAAAIGMYAVASGVYTHLGLPPNILGSQTVTDLALNG
LEGLVGASFAVEPDPLKAADLLDARIRAKREALGLKP
SEQ ID NO: 50-Desulfoluna spongiiphila Carbon monoxide
dehydrogenase (SAMN05216233)
MAREKRKIEELSMWEDARAMIRKAQAEGIETVWDRLEEQTPHCRFCELGTTCRNCTMGPCRISPKK
GGKMQRGVCGADADVVVARNFGRFIAGGAAGHSDHGRDLIEVLAGVAEGECPDYGIRDKAKLVRIA
EELGIKSEGRSEMDIAGDLAEVLFGDFGSRKAEVAFLKRAPESRRETWKQLGMTPRGVDREIAEMM
HRTHMGCDNDAPSTLIHAARTALADGWAGSMIGTELSDIIFGTPTPSESTANLGVLKKDQVNILVH
GHNPVVSEMILAAARMPELVARAKDVGASGINVGGLCCTGNELLMRQGIPMAGNHLMTELSIVTGA
VEAVVVDYQCIMPSMVQISGCYHTRFITTSGKARFTGATHFDIHPENAMEKAREIVALAVDAFAER
DASRVEIPHEPVPIMTGFSNEAILAAVGGTPDPLIDAMKRGAIRGVVGIVGCNNPKFTQDSMNVGL
AKALLKKDILVLVTGCVTTAAGKAGLLMPEGAEMAGPGLKEVCGALGIPPVLHMGSCVDNARILQL
CALLAETCGVDISDLPVAASSPEWYSEKAAAIGLYAVASGIETHLGHPPNILGSDTVTHLATEGLE
DLVGARFIIEKDPEEAAERLDKRITAKRVGLGWNP
SEQ ID NO: 51-Halodesulfovibrio spirochaetisodalis Carbon
monoxide dehydrogenase (SP90)
MAKEPKKPEELSIWQDAHAMIRKAKAESIETAWDRLERQTPHCKFCDLGTTCRNCIMGPCRISTKP
GAKMNLGVCGADADVVVARNFGRFIAGGSAGHSDHGRDLIEVLEAIVDGDTKHYTITEPEKLIRLA
AEVGIATEGRELNDVANDLVDECYKDFGSRRSSLAFLSRAPEQRRALWDRVGITPRGVDREIAEMM
HRTHMGCDNDAPNTLLHSARCALSDGWGGSMIGTELSDVIFGTPTPSRSTSNLGVIKEDKVNILVH
GHNPVVSEMILAAARRPELVEKAKAAGAAGINVAGLCCTGNELLMRQGIPMAGNHLMTELAIVTGA
VEAVVVDYQCIMPSLVTAAHCYHTRFITTAEKAKFTGAMHFEVHPHNALEQATAIVEEAIRAYTER
DKGRVEIPAESVEIMTGFSNEAILSALGGTLTPLLDAIKAGKIRGIVGIVGCNNPKIKQDSANVGL
AKELLKRDILVLVTGCVTTAAGKAGLLVPEGIEMAGEGIKEVCGALGIPPVLHLGSCVDNSRIMHL
CGLVAKELGVDISDLPVGASSPEWYSEKAAAIGMYAVASGIMTHLGLPPNILGSETVINIALEGLE
DIVGAHFVVEDDYVKAAELLDARIRMKRVGLGLSE
SEQ ID NO: 52-Desulfovibrio desulfuricans Carbon monoxide
dehydrogenase (Ddes)
MDDRQTDINNMSIWEDAQKMLHKARAEGIETAWDRLAQQTPHCRFCELGTTCRNCVMGPCRISAKA
APGGKLSRGVCGADAHVIVARNFGRFIAGGSAGHSDHGRDVIETLEAVVEGKAEGYEIRDPAKLLR
IAGELGIAVDGLAVNEVAALVVDACYSDFGSRRGAVNFISRVPAKRRQVWEKLGITPRGVDREIAE
MMHRTHMGCDNDAPNTMLHAARCALSDGWAGSMIATELCDILFGTPSPRMSTVNLGVIKKETVNIL
VHGHNPVVSEMILAVSREPEVLEQARQAGAAGITVAGLCCTGNELLMRQGIPMAGNHLMTELAIVT
GAVEAVVVDYQCIMPSLVQVAACYHTRFIDTAPKARFTGAVHMNFTPENARQEASAILHMAIEAFA
ARDPGRVDIPVSPVNIMTGFSNEAILEALGGSLDPLLDAVKAGTVRGFAAVVGCNNPKVRQDSANV
GIIKELIKKDIMVLVTGCITTAAGKAGLLLPEGAAMAGPGLQKLCASLGIPPVLHMGSCVDNARIM
HLCGLIANSLGVDISDLPVVASAPEWYSEKAAAIGLYAVASGVYTHLGLPPNILGSDVVTDIALNG
LEGLVGASFVVEADPVKAADLLDRRIRTKRSALGLDA
SEQ ID NO: 53-Desulfurivibrio alkaliphilus Carbon monoxide
dehydrogenase (DaAHT2)
MAEKNKKSIEEKSLWEDARAMLRKAEQDGVETAWDRLEQQTPHCKFCELGTSCRNCVMGPCRITAK
MPRGVCGADADVVVARNFGRFIAGGSAGHSDHGRDLIEVLEAIVEGETKDYQIKDEAKLRRIAGEL
GVADAGGGALAAVARAVMEILYADFGSRKKEVAFLGRVPEKRRAIWQSLGMTPRGVDREIAEMMHR
THMGCDNDAASTLIHAARTALADGWAGSMIGTELSDVIFGTPSPKKSTVNLGVINQEQVNILVHGH
NPVVSEMIVAAANDPEMLARAEAAGAGGINVAGLCCTGNEILARRGIPMAGNHLMTELAVVTGAVE
AVVVDYQCIMPSLLQAAGCYHTQFITTANKARFSGAVHFDIKPATALAQAKEIVQLAIDGFSKRDP
QRVEIPSQPVEIMTGFSNEAILNALGGSLTPLLEAIKAGKIRGVVGIVGCNNPKFQQDSGNIGLTK
ALIQRDILVLVTGCVTTAAGKAGLLLPQAIDQAGPGLQEICGALGIPPVVHFGSCVDNSRIIHLCA
LIAEELGVDISDLPVAASSPEWYSEKAAAIGLYAVASGIYTHLGLPPNILGSPAVTDLALNGLESL
VGASFVVEPDPDKAADLLEARIVSKRKALGLPK
SEQ ID NO: 54-Pseudodesulfovibrio aespoeensis Carbon monoxide
dehydrogenase (Daes)
MAKEPRPVEELSIWEDARAMIRKARKEGIETVHERLDQQTPHCKFCELGTTCRNCTMGPCRISAKA
PRGVCGADADVVVARNFGRFVTGGAAGHSDHGRDLIEVLEAIVEGETKDYRITDEPKLRAIAAEIG
VATEGRTVMEVARDVMDAFYADFGSRRKSISFLCRVPEKRKAIWAKLGMTPRGVDREIAEMMHRTH
MGCDNDAPNTMIHAARTALADGWGGSMIGTELSDVIFGTPTPKMSTANLAVIKKDQVNILVHGHNP
VVSEMILAAAREPELIEQAKKLGATGINVAGLCCTGNELLMRQGIPMAGNHLMTELAIITGAVEAV
VVDYQCIMPSLVQISGCYHTKFIDTAAKARFTGAIHFDIHPHNAMEQARKIVGLAVQGFVERDPGR
VDIPGEPVDIMTGFSNEAVIKALGGSLDPLIQAIASGDIRGAVGIVGCNNPKFKQDSMNVGLAKEL
IKKDILVLVTGCVTTAAGKAGLLLPSAIDQAGPGLRKICGSLGIPPVLHYGSCVDNARILQLCAAL
ANGLGVDISDLPVGASSPEWYSEKAAAIGLYAVASGIYTHLGHPPNILGSETVTNLAVSGLEDLVG
ACFVIEPDPVKAAELFDIRIRAKRKGLGLSE
SEQ ID NO: 55-Desulfovibrio alaskensis Carbon monoxide
dehydrogenase (Dde_3028)
MAKEPRPVEALSIWEDARAMLTKARAEGIETAHERLQQQTPHCKFCELGVSCRNCTMGPCKITPKA
PRGVCGADADVIVARNFGRFVAGGSAGHSDHGRDLVEVLESIVEGSTSDYRITDEAKLRRIAAELG
VTVEDRSTMDIAAELIDIFYADFGSRKKEVAFLCRVPAKRRELWDRLGMTPRGVDREIAEMMHRTH
MGCDNDAPNTLIHAARTALADGWAGSMIGTELSDVIFGTPQPSMSHANLAAIKKDKVNILVHGHNP
VVSEMILAAAREPALIAEAEAMGAQGINVAGLCCTGNELLMRQGIPMAGNHLMTELAIVTGAVEAV
VVDYQCIMPSLVQIAGCYHTLFIDTAQKARFTGAVHFDIHPHNALEQAREIVRMAVQAYARRDASR
VQIPGEPVNIMTGFSNEAVIAALGGSLDPLVQAIAAGDIRGAVGIVGCNNPKFQQDSMNVGLAKEL
IKKDILVLVTGCVTTAAGKAGLLLPEAAEQAGPGLRKICGALGIPPVLHYGSCVDNSRILQLCAAL
ANALGVDISDLPVGASSPEWYSEKAAAIGLYAVASGIYTHLGHPPHILGSQTVTDLAVSGLEDLVG
ASFFIEPDPVKAALAFDLRIRAKRKALGLGE
SEQ ID NO: 56-Desulfovibrio ferrophilus Carbon monoxide
dehydrogenase (DFE_2686)
MAKELKPPQELSIWEDAQKMIEKARRDGVETVWDRYEQQSPHCTFCELGLICKNCNMGPCRISPKE
GGKMQRGVCGADAHVIVARNFGRFVAGGAAGHSDHGRDLIEVLEAIVEGKAPGYQISDEAKLRRVS
AELGINVDGRDVMDVARDLMDICYADFGSRKKEVGFTCRVPEKRKEIWRKLGIMPRGVDREIAEMM
HRTHMGCDNDAPNTLLHAARTSLADGWAGSMIGTELSDIIFGTPAPSASRANLGVLKADHVNLLVH
GHNPVVSEMVLAAAREPEMIAKAKAAGAAGINIGGLCCTGNELLMRQGIPMAGNHLMTELAIVTGA
VDAMVVDYQCIMPSLVQTAACYHTKFITTADKAKFTGATHVSFHPDNAVKQARVVVEMAIEAYERR
DQGRVEIPCEPVDITTGFSIEALLSALGGTLDPVLDAVKAGKIRGFTAIVGCNNPRIKHDSANVGL
AKALIKKDILVLATGCVTTAAGKAGLLMPEGASMAGPGLQEVCGALGIPPVLHMGSCVDNSRVIQL
CAAIANALGVDISDLPVWGASPEWYSEKAVAIGLYCVASGIPVQIGTPPHITGSDVVTNLALSGLE
DLVGAAFLVEADPEKAADIMDERIKAKRKALGLSE
SEQ ID NO: 57-Carboxydothermus hydrogenoformans Carbon monoxide
dehydrogenase 2 (cooS2)
MAKQNLKSTDRAVQQMLDKAKREGIQTVWDRYEAMKPQCGFGETGLCCRHCLQGPCRINPFGDEPK
VGICGATAEVIVARGLDRSIAAGAAGHSGHAKHLAHTLKKAVQGKAASYMIKDRTKLHSIAKRLGI
PTEGQKDEDIALEVAKAALADFHEKDTPVLWVTTVLPPSRVKVLSAHGLIPAGIDHEIAEIMHRTS
MGCDADAQNLLLGGLRCSLADLAGCYMGTDLADILFGTPAPVVTESNLGVLKADAVNVAVHGHNPV
LSDIIVSVSKEMENEARAAGATGINVVGICCTGNEVLMRHGIPACTHSVSQEMAMITGALDAMILD
YQCIQPSVATIAECTGTTVITTMEMSKITGATHVNFAEEAAVENAKQILRLAIDTFKRRKGKPVEI
PNIKTKVVAGESTEAIINALSKLNANDPLKPLIDNVVNGNIRGVCLFAGCNNVKVPQDQNETTIAR
KLLKQNVLVVATGCGAGALMRHGFMDPANVDELCGDGLKAVLTAIGEANGLGGPLPPVLHMGSCVD
NSRAVALVAALANRLGVDLDRLPVVASAAEAMHEKAVAIGTWAVTIGLPTHIGVLPPITGSLPVTQ
ILTSSVKDITGGYFIVELDPETAADKLLAAINERRAGLGLPW
SEQ ID NO: 58-Desulfofundulus salinum Carbon monoxide
dehydrogenase (cooS)
PMSDLTGNTKTNSLKGGSTETDHSKKSTDPAVQQMLEKAAREGIQTVWDRYQAMRPLCRFGETGLC
CRHCLQGPCRINPLGREPRVGICGATADVIVIRGLDRAIAAGAAGHSGHAKHLAHTLKKAAEGRAP
DYLVKDKEKLHSVARRLGIVTDGRPENEIALDVARAALADFHEKDTPVTWVTSVLPPDRLEVESRH
GLVPAGIDHEIAEVMHRTSMGCDADASNLLLGGLRCALGDLAGCYMATDLSDILFGTPSPVVTECN
LGVLKAGAVNVAVHGHNPVLSDIIVLVAKEMEAEARAAGAQGINVVGICCTGNEVLMRHGISPCTH
SVSQEMAIITGALDALVVDYQCIQPAVATVAECLGTTVITTMDMAKITGTTHVDFSEETAVENARQ
ILRLAIESFTRRRGKPVEIPAMKKKVFAGFSVEAILSALGRLDAGDPLKPLIDHLTAGNIRGVCLF
AGCNNVKVPQDYNFITTARKLLEQDVLVLASGCSAGALMRHGFMDPASVEELCGGGLKAVLTAIGE
ANGLGGPLPPVLHVGSCVDNSRAAALAVALAGRLGVDLDRLPVVASAAEAMHEKAVSIGAWAVALG
LPTHVGVMLPVSGGPLVHRILTEEVKGLTGGYFILEPDPESATEKLLEAINERRAGLGLHY
SEQ ID NO: 59-Caldanaerobacter subterraneus tengcongensis Carbon
monoxide dehydrogenase (TTE1708)
MTKQCKVSLDPAVCEMVEKARRVKVETVWDRYQAMLPQCGFGETGLCCRHCLQGPCRIDPFGGEPK
LGICGATADVIVARGLDRAIAAGAASHSGHARHLAHTLKLAAKGKARDYTIKDKAKLRSVAARLGI
PTEGRSIAEIALDVAEAALADFHEKDTPVMWAATTVTKKRARLFEEKGLLPKGIDYEVSDIMHRTS
YGVDADPVNLLLGGLRCGLADLAGCYMGTDMADILFGTPQPVVTEANMGVLKADAVNVAVHGHNPV
LSEVIVAVAKEMEAEAKAAGASGINVVGICCTGNEVLMRHGIPACTHSVSQEMALVTGALDAMVVD
YQCIMPSLATVAECMGTKLITTMEIAKIPGAIHIEFSEEKAGEKAREILRLAIETFTRRRGKPVDI
PPYKTKVVAGFSVEAIVKALSKLNAEDPLKPLIDQIAAGNIRGVCLFAGCNNVKVPQDQNFTAIAR
RLLKENVLVLATGCGAGALMRHGFMDPANVGELCGEGLKAVLTAIGEANGLGGPLPPVLHVGSCVD
NSRAVALAVAVADRLGVDTDQLPVVASAAEAVAEKAVSIGTYAVALGLPTHVGVMLPVLGGPLVTK
VLTDKVKELTGGYFIVDLDPESAAEKLLAAIDERRAALGLSVPGGGRR
SEQ ID NO: 60-Thermanaeromonas toyohensis Carbon monoxide
dehydrogenase (SAMN00808754_0706)
MANPCKVSLDPAVCEMVEKARRVKVETVWDRYQAMLPQCGFGETGLCCRHCLQGPCRIDPFGGEPK
TGICGATADVMVARGLDRAIAAGSAAHSGHARHLAHTLKLAAKGKARDYTVKDKAKLRSVAARLGI
PTEGRSIAEIALDVAEAALADFHEKDTPVMWVATTVTKKRAKLFAEKGLLPKGIDYEVADIMHRTL
YGTDADPVNLLLGGLRCGLADLAGCYMGTDLSDILFGTPQPVVTEANMGVLKADAVNVAVHGHNPV
LSDVIVAVAKEMESEAKAAGASGVNVVGICCTGNEVMMRHGIPACTHSVSQEMALVTGALDAVVVD
YQCIMPSLATVAECMGTKLITTMEITKIPGAIHIDFSEETAGENARQIIRLAIESFSRRRGKPVDI
PQIKTKVVAGFSVEAIINALAKLNAEDPLKPLIDQIVAGNIRGVCLFAGCNNVKVPQDKNFTAIAR
RLLKENVLVLATGCGAGALMRHGFMDPANVGELCGEGLKAVLTAIGEANGLGGPLPAVLHVGSCVD
NSRAVAVAVAVADRLGVDTDQLPVVASAAEAVSEKAISIGTYAVAIGLPTHVGVMLPVVGGPLVTK
VLTEKVKDLTGGYFIVDPDPESAAKKLLAAIDERRAALGLSVPGGGRR
SEQ ID NO: 61-Desulfobulbus sp. Carbon monoxide dehydrogenase
(Gene: JT06_17280)
MDKSRDPAVQQMIARAEAQNVTTVWDRYAAMTPQCGFGDTGLCCRHCLQGPCRIDPFGEGPREGIC
GASADVMVARGLDRAIAAGTAAHSGHARHLAHTLKKMAMGKAEAYAVKEPGKLEAVAARLGIPTEG
RQEREIALDVADAALADFHEKDTPVLWAATVVNPERAKVLADLNLVPKGIDHEVSEIMHRTLYGVD
ADPVNLLLAGLRCGVADLAGCWMGTDLADILFGIPRPTVSSANLGTLRADAVNIALHGHNPVLSEI
LVGAVGAKEEAARAAGATGINLVGICCTGNEVMMRHGIPACTHSVSQEMAIMTGAVDAMVVDYQCI
MPSVVNVAECTGTRIITTMDIAKITGATHVDFSEEAAAAKAHEIIDQAIDQFRRRRNTPVEIPAVK
TPVVAGFSVEAIVAALSGIDAEQPLKPLVDSIKSGAIRGVCLFAGCNNVKVPQDRNFVRMARRLLR
ENVLVVATGCGAGALMRHGFMDPANVEALCGDGLKAVLTAVGLANDLGGPLPPVLHMGSCVDNSRA
VALTVAVAQYLGVDTHQLPIVASAAEAVSEKAVSIGVYAVAAGLPTHVGVMLPVLGSRLVTRVLTD
KVKDLTGGYFIVDLDPDSAADKLLAAIDERRRGLGLG
SEQ ID NO: 62-Desulfotomaculum copahuensis Carbon monoxide
dehydrogenase (A6M21_00615)
MQSADPAVREMLDEARRRGVETVWDRYAAQLPQCGFGELGVCCRNCMQGPCRVSPFDDGPARGVCG
ATADTMVARNLVRAIAAGTAAHSGHAKHLAHTLKKSVQGKAPDYPVRDEAKLRAVAARQGLEADGV
PAAELAARMAEAALANFSEGEEPLAWTTATVTAGRVQAWEKLGVLPVGIDSIISEIMHRTHLGVDA
DAVNLLLGGVACAVADYTGCHLGTDLADILFGTPQPVVSEANMGVLKADQVNIALHGHNPVLSEVI
VQVARQMEDEAKAAGAAGINLVGVCCTGNEVLMRHGIPPATHSISQELPMLTGALDAMVVDYQCVY
PSLVGVAECTGTKIITTMGMARITGATHVDFEEENAAAGARQIIRLAIEAFGARRGRPVQIPQYKS
QVVAGFSVEAIVAALSKLDAADPLKPVIDNIVAGNIQGVCLFAGCNNVKVPQDENYLVMARELAAR
DVLLLATGCGAGAYARHGYLTPEAVGEYAGPGLKAVLTAVGEANGLGGPLPLVLHMGSCVDNSRAV
DLAVAVANRLGVDTAQLPVVASAPEFKTEKAVAIGTWAVACGLPTHLGLVPPVLGSATVTGLLTGG
IKDLLGGYFIVETDPVKAAEKLFAAIQERRRGLNLATRSW
SEQ ID NO: 63-Pelotomaculum propionicicum Carbon monoxide
dehydrogenase (cooS2)
MRSRDSAVSLMLEAAQKEAIETTWDRLDAQLPQCGFGELGVCCRHCMQGPCRISPFEDGPQRGICG
ATADTMVARGLVRAIAAGAAAHSGHAKHLAHTLHKWVKGQAPDYNVKNEAKLRAVAARQGISADGL
SVRELAAKVVASVFSQFAEGEEPLAWAGATVTTGRVEALAKLGALPVGIDSSIAEMMHRTHLGVDA
DPVNLLLGGISCAIADYTGCHIGTDLADILFGTPVPVVSEANMGVLKENSVNIAVHGHNPVLSEVI
VRIAGEMQDEAKAAGADGIVLVGICCTGNEVLMRHGIPPVTHGVSQELPILTGALDAIVVDYQCVY
PSIVEVARCYGTKVITTMSIAKIQGSTHLEFVEEDAAGKAKEIIGVAIEAFKARKGRPVNIPQVKS
KVVAGFSVEAIVAALAKLDAADPLKPVIDNIVNGNIQGVVLFAGCNNVKVPQDHNYLVMAEELAKR
DVLLLATGCGAGAYARQGLMTPEATEKYAGPGLKIVLTAIGEANGLGGPLPLVLHMGSCVDNTRAV
DLTVAIANKLGVDIDKLPVAASAPEFKTEKAVAIGTWAVASGLPVHLGLVPPVLGGPQVTSILTAG
IKDLLGGYFIVETDPQKAAEKLFAAIQERRAGLGLGTRQW
SEQ ID NO: 64-Methylomusa anaerophila Carbon monoxide
dehydrogenase (cooS2)
MEAMKAKKAVKATDCAVGEMLEVARKQGIETVWDRYARQIPQCGFGDSGLCCRNCTQGPCRIDPFG
DGPAAGVCGITADSMVARNLIRAIAAGAASHSGHAKHLAHVLKKVAAGKLTDYSVKDENKLRAVAT
RLGIPTEGKSVQELAAAVASAALEEFSEREEPLAWAATTLTSSQVEKLGGLGVLPAGIDSAISEIL
QRTTLGVDADPVNLLLGGVKGAVADFAGCHLGTDLADILFGTPTPVFSEANMGVLKENAVNIALHG
HNPVVSELIVVAARQEDILAQAKSAGAEGINLVGICCTGNEVLMRQGVAPLTHSLSQEMPILTGAL
DAIVVDYQCVYPSLANIADCYGTKLITTMDIAKIPGAVHMAIEEERGLEQAKEIIKLAVERFKARK
GAPTCIPQVKQKVIAGFSTEAIIGALSKLNAADPLKPLIDNIVNGNIQGVVLFAGCNNVKVPQDAN
FIAIVKELAKRDVLMLATGCGAGALARHGLLTSEATMEYAGPGLKAVFTAIGEANGLGGPLPLVLH
VGSCVDNSRPVDIAVALANKLGVDVNQLPVAASAPEFKAEKAVAIGTWAVTLGLPTHLGLVPPVLG
SKLVTRVLTQDIKDITGGYFLVETDPLLAAEKIFDALQERRQKLNIGTRRW
SEQ ID NO: 65-Sporomusa silvacetica Carbon monoxide
dehydrogenase (cooS2)
MKSQEPLDSSVKEMLDVAKTSNVSTVWDRYQSQLPQCGFGESGICCRNCLQGPCRISPFGDGPDVG
VCGASADTIVARNLIRGIAGGAASHSGHGKHLAHVLKKVAKGEALAYGVKEAAKLNAVAARLGIPT
ENRSTKEIAGDVAQAALDEFSERETPNAWAATTLTAGRVAKLNELKVMPYGIDSIIAEIMHRTTLG
VDADPVNLLLGGIKGAIADFAGCHLGTDLADILFGIPAPSVSEANMGVIKEKAVNIALHGHNPLLS
DMIVATSREPEILAEAKAAGAEQGIQLMGICCTGNEVLMRHGVPTVTTAVSQELPILTGAIDAMIV
DYQCVYPSLPTVAACFGTPLITTMDIAKIPGATHIEFDEDHASEKAREIIRIAITAFKRRCNKPVH
IPPKTSKVVAGFSTETIVGALAKLNADDPLQPLVDNIVNGNIQGVVLFAGCSNIKVGHDQNFVAIA
KELIARNILVLATGCGAGAFARNGLLDPQATLDYAGDGLKAVLTAIGEANGLNGPLPPILHMGSCV
DNSRPVDIAVALANKLKVDIPQLPVVASAPEYKTEKALAIGTWAATLGLPTHLGLVPPVVGSKLVT
RILTQDIKELTGGYFIVEPDPILAAEKIFNAIQERRQNLNISTRRW
SEQ ID NO: 66-Heliobacillus mobilis Carbon monoxide
dehydrogenase (cooS)
MDTRSVDLAVQEMLEVALQNKIETAWDRLNFQQPQCGFGELGLCCRNCTQGPCRIDPFGDGPRKGV
CGADADTLVARNLARSIVAGTASHSGHSKHLAHVLKKWADGKAPDYPVKDERKLLAVAQRHGIEVE
GKEIKEIARSVAEVALEDFSERNTPIGFANRTVTKNRAELWNKFGVNPVGIDSIIAEMMHRTTLGV
DADANNLLLGSIAAGIADYAGCRMGTDLSDILFGTPAPVISEMNMAVLDPKQVNVALHGHNPVLSD
LLVQVSREMREEAKMAGATGINLVGICCTGSEVLMRHGVPSCTHSVSQELPILTGVLDAMVVDYQC
VYPSIATLAGCYSTKVITTMSMAKIPGAIHIEIHEEGAWAKGKEILRLAIEAFKERAGRLYQIPRY
KEKVVAGFSKEAIVAALAKLNQEEPLQPLIDNIVNGNIQGICLFAGCNNVKVRQDENYIIMAKELA
KRNVLLLATGCGAGSLGRLGFMNGEATKAYAGDGMKDVLSAIGEANGLEGPLPPVLHLGSCVDNSR
AVDIAVAIAEKLGVDTDMLPVVASAPEYKTEKAFSIGTYAVALGLPTHLGMVPPVAGSKQVVKTLT
KDIREITGGYFIVETNPSEAAKKLFLAIQERRAGLGLLTRLW
SEQ ID NO: 67-Desulfocucumis palustris Carbon monoxide
dehydrogenase (DCCM_2691)
MKSRDKAVNQMIKAAGEREVETVWDRYQTQQPQCGFGELGVCCRNCTQGPCRINPYGDGPDHGVCG
ANADTIVARNLVRAIAAGAAAHSGHARHLAHALKKGLAGKAPAYGLKDEAKLRALAARVGLDPELP
AEELVNKFMDMALEQFAEGEGPLKWAQSELTGGCGETFERLGTMPTGIDNTIVEMLHRTHLGVDAD
PVNLLLAGVSCAIADYAGSHLATDLADIMFGTPGPVFSQANMGVMKEKSVNIALHGHNPVLSDIIV
QEAREMEEEARGAGAEGINLMGVCCTGNEILMRHGVPALTHSISQEMPMLTGALDAMVVDYQCVYP
SLAGVAECTGTKLITTMDMAKISGAVHLGMHEENAAEKAREIISVAIEQFRARQGRPVNIPSVSSK
VVAGFSVEAIVQALSKLDAADPLKPVIDSLAAGSLQGVVLLAGCNNVKVPQDDSFLVIARELAARD
VLLLATGCGAGAYARHGYLTPEATLEYAGPGLKAVLTAIGEANGLGGPLPLVLHMGSCVDNSRAVD
LAVAVADKLGVDLNKLPVVASAPEFKTEKAVSIGTYAMAIGLPVHLGVVPPVLGSRLVTSVLTEGV
KDLFGGYFIVETDPYKAAGAIFAAIQERRSGLGLPTREWE
SEQ ID NO: 68-Escherichia coli Formate dehydrogenase, nitrate-
inducible, major subunit (fdnG)
MDVSRRQFFKICAGGMAGTTVAALGFAPKQALAQARNYKLLRAKEIRNTCTYCSVGCGLLMYSLGD
GAKNAREAIYHIEGDPDHPVSRGALCPKGAGLLDYVNSENRLRYPEYRAPGSDKWQRISWEEAFSR
IAKLMKADRDANFIEKNEQGVTVNRWLSTGMLCASGASNETGMLTQKFARSLGMLAVDNQARVUHG
PTVASLAPTFGRGAMTNHWVDIKNANVVMVMGGNAAEAHPVGFRWAMEAKNNNDATLIVVDPRETR
TASVADIYAPIRSGTDITFLSGVLRYLIENNKINAEYVKHYTNASLLVRDDFAFEDGLFSGYDAEK
RQYDKSSWNYQLDENGYAKRDETLTHPRCVWNLLKEHVSRYTPDVVENICGTPKADFLKVCEVLAS
TSAPDRTTTFLYALGWTQHTVGAQNIRTMAMIQLLLGNMGMAGGGVNALRGHSNIQGLTDLGLLST
SLPGYLTLPSEKQVDLQSYLEANTPKATLADQVNYWSNYPKFFVSLMKSFYGDAAQKENNWGYDWL
PKWDQTYDVIKYFNMMDEGKVTGYFCQGFNPVASFPDKNKVVSCLSKLKYMVVIDPLVTETSTEWQ
NHGESNDVDPASIQTEVFRLPSTCFAEEDGSIANSGRWLQWHWKGQDAPGEARNDGEILAGIYHHL
RELYQSEGGKGVEPLMKMSWNYKQPHEPQSDEVAKENNGYALEDLYDANGVLIAKKGQLLSSFAHL
RDDGTTASSCWIYTGSWTEQGNQMANRDNSDPSGLGNTLGWAWAWPLNRRVLYNRASADINGKPWD
PKRMLIQWNGSKWTGNDIPDFGNAAPGTPTGPFIMQPEGMGRLFAINKMAEGPFPEHYEPIETPLG
TNPLHPNVVSNPVVRLYEQDALRMGKKEQFPYVGTTYRLTEHFHTWTKHALLNAIAQPEQFVEISE
TLAAAKGINNGDRVTVSSKRGFIRAVAVVTRRLKPLNVNGQQVETVGIPIHWGFEGVARKGYIANT
LTPNVGDANSQTPEYKAFLVNIEKANOTE: highlighted U represents a
selenocysteine encoded by opal stop codon
SEQ ID NO: 69-Shigella flexneri Formate dehydrogenase-N,
nitrate-inducible, alpha subunit (fdnG)
MDVSRRQFFKICAGGMAGTTVAALGFAPKQALAQARNYKLLRAKEIRNTCTYCSVGCGLLMYSLGD
GAKNAREAIYHIEGDPDHPVSRGALCPKGAGLLDYVNSENRLRYPKYRAPGSDKWQRISWEEAFSR
IAKLMKADRDANFIEKNEQGVTVNRWLSTGMLCASGASNETGMLTQKFARSLGMLAVDNQARVUHG
PTVASLAPTFGRGAMTNHWVDIKNANVVMVMGGNAAEAHPVGERWAMEAKNNNDATLIVVDPRETR
TASVADIYAPIRSGTDITFLSGILRYLIENNKINAEYVKHYTNASLLVRDDFAFEDGLESGYDAEK
RQYDKSSWNYQFDENGYAKRDETLTHPRCVWNLLKAHVSRYTPDVVENICGTPKADFLKVCEVLAS
TSAPDRTTTFLYALGWTQHTVGAQNIRTMAMIQLLLGNMGMAGGGVNALRGHSNIQGLTDLGLLST
SLPGYLTLPSEKQVDLQSYLEANTPKATLADQVNYWSNYPKFFVSLMKSFYGDAAQKENNWGYDWL
PKWDQTYDVIKYFNMMDEGKVTGYFCQGFNPVASFPDKNKVVSCLSKLKYMVVIDPLVTETSTFWQ
NHGESNDVDPASIQTEVFRLPSTCFAEEDGSIANSGRWLQWHWKGQDAPGEARNDGEILAGIYHHL
RELYQAEGGKGVEPLMKMSWNYKQPHEPQSDEVAKENNGYALEDLYDANGVLIAKKGQLLSSFAHL
RDDGTTASSCWIYTGSWTEQGNQMANRDNSDPSGLGNILGWAWAWPLNRRVLYNRASADINGKPWD
PKRMLIQWNGSKWTGNDIPDFGNAAPGTPTGPFIMQPEGMGRLFAINKMAEGPFPEHYEPIETPLG
TNPLHPNVVSNPVVRLYEQDALRMGKKEQFPYVGTTYRLTEHFHTWTKHALLNAIAQPEQFVEISE
TQAAAKGINNGDRVTVSSKRGFIRAVAVVTRRLKPLNVNGQQVETVGIPIHWGFEGVARKGYIANT
LTPNVGDANSQTPEYKAFLVNIEKA
SEQ ID NO: 70-Enterobacteriaceae bacterium Formate
dehydrogenase-N subunit alpha (fdnG)
MDVSRRQFFKICAGGMAGTTVAALGFAPKQALAQARNYKLLRAKEIRNTCTYCSVGCGLLMYSLGD
GAKNAREAIYHIEGDPDHPVSRGALCPKGAGLLDYVNSENRLRYPEYRAPGSDKWQRISWEEAFSR
IAKLMKADRDANFIEKNEQGVTVNRWLSTGMLCASGASNETGMLTQKFARSLGMLAVDNQARVUHG
PTVASLAPTFGRGAMTNHWVDIKNANVVMVMGGNAAEAHPVGFRWAMEAKNNNDATLIVVDPRFTR
TASVADIYAPIRSGTDITFLSGVLRYLIENDKINAEYVKHYTNASLLVRDDFAFEDGLFSGYDAEK
RQYDKSSWNYQFDENGYAKRDETLTHPRCVWNLLKAHVSRYTPDVVENICGTPKADFLKVCEVLAS
TSAPDRITTFLYALGWTQHTVGAQNIRTMAMIQLLLGNMGMAGGGVNALRGHSNIQGLTDLGLLST
SLPGYLTLPSEKQVDLQSYLEANTPKATLAGQVNYWSNYPKFFVSLMKSFYGDAAQKENNWGYDWL
PKWDQTYDVIKYFNMMDEGKVTGYFCQGFNPVASFPDKNKVVSCLSKLKYMVVIDPLVTETSTFWQ
NHGESNDVDPASIQTEVFRLPSTCFAEEDGSIANSGRWLQWHWKGQDAPGEARNDGEILAGIYHHL
RELYQAEGGKGVEPLMKMSWNYKQPHEPQSDEVAKENNGYALEDLYDANGVLIAKKGQLLSSFAHL
RDDGTTASSCWIYTGSWTEQGNQMANRDNSDPSGLGNTLGWAWAWPLNRRVLYNRASADINGKPWD
PKRMLIQWNGSKWTGNDIPDFGNAAPGTPTGPFIMQPEGMGRLFAINKMAEGPFPEHYEPIETPLG
TNPLHPNVVSNPVVRLYEQDALRMGKKEQFPYVGTTYRLTEHFHTWTKHALLNAIAQPEQFVEISE
TLAAAKGINNGDRVTVSSKRGFIRAVAVVTRRLKPLNVNGQQVETVGIPIHWGFEGVARKGYIANT
LTPNVGDANSQTPEYKAFLVNIEKA
SEQ ID NO: 71-Salmonella typhimurium Molybdopterin
oxidoreductase (fdnG)
MDVSRRQFFKICAGGMAGTTVAALGFTPKMALAQARNYKLLRAKEIRNSCTYCSVGCGLLMYSLGD
GAKNAKEAIYHIEGDPDHPVSRGALCPKGAGLLDYVHSEDRLRYPEYRAPGSDKWQRISWDDAFTR
IAKLMKADRDANFIEKNEQGVTVNRWLSTGMLCASAASNETGMLTQKFARSLGMLAVDNQARVUHG
PTVASLAPTFGRGAMTNHWVDIKNANVVMVMGGNAAEAHPVGFRWAMEAKNNNDATLIVVDPRETR
TASVADIYAPIRSGTDITFLSGVLLYLIENNKINAEYVKHYTNASLLVRDDFAFDDGLESGYDAQK
RQYDKSSWNYQFDENGYAKRDETLTHPRCVWNLLKQHVSRYTPDVVENICGTPKADFLKVCEVLAS
TSVPDRITTFLYALGWTQHTVGAQNIRIMAMIQLLLGNMGMAGGGVNALRGHSNIQGLTDLGLLST
SLPGYLTLPSEKQADLQTYLAANTPKATLADQVNYWGNYPKFFVSLMKSFYGDAAQQENDWGFAWL
PKWDQSYDVIKYFNMMDSGKVTGYFCQGFNPVASFPDKNKVVQSLSKLKYLVVIDPLVTETSTEWQ
NHGESNDVDPTTIQTEVFRLPSTCFAEEDGSIANSGRWLQWHWKGQDAPGEARNDGEILAGIYHRL
REMYRAEGGKGAEPLLKMSWNYKQPDEPHSEEVAKENNGYALEDLYDANGTLLARKGQLLSSFALL
RDDGTTSSSCWIYTGSWTEQGNQMSRRDNADPSGLGNTLGWAWAWPLNRRVLYNRASADPQGKPWD
PKRMLIQWNGAKWTGNDIPDFNNAAPGSGTNPFIMQPEGLGRLFAIDKMAEGPFPEHYEPMETPLG
TNPLHPNVVSNPAARLYEEDALRMGKKEQFPYVGTTYRLTEHFHTWTKHALLNAIAQPEQFVEISE
TLAAAKGIANGDYVKVSSKRGFIRAVAVVTRRLRTLHVNGQQVETVGIPIHWGFEGVARKGYIANT
LTPNVGDANSQTPEYKAFLVNIEKA
SEQ ID NO: 72-Citrobacter rodentium Formate dehydrogenase,
nitrate-inducible, major subunit (fdnG)
MDVSRRKFFKICAGGMAGTTVAALGFTPKMALAQARNYKLLRAKEIRNTCTYCSVGCGLLMYSLGD
GAKNTKEAIYHIEGDPDHPVSRGALCPKGAGLLDYVHSENRLRYPEYRAPGSDKWQRISWDEAFSR
IAKLMKADRDANFIEKNEQGVTVNRWLSTGMLCASAASNETGMLTQKFVRSLGMLAVDNQARVUHG
PTVASLAPTFGRGAMTNHWVDIKNANVVMVMGGNAAEAHPVGFRWAMEAKNNNDATLIVVDPRETR
TASVADIYAPIRSGTDITFLSGVLLYLIENNKINAEYVKHYTNASLLVRDDFAFEDGLFSGYDADK
RQYDKSSWNYQFDENGFARRDETLSHPRCVWNLLKQHVSRYTPEVVENICGTPKADFLKVCEVLAS
TSAADRTTTFLYALGWTQHTVGAQNIRTMAMIQLLLGNMGMAGGGVNALRGHSNIQGLTDLGLLST
SLPGYLTLPSEKHSDWQSWLNANTPKATQADQVNYWSNYPKFAVSLMKAFYGDDAQKENDWGEDWL
PKWDQAYDVIKYFNMMDSGKVTGYICQGFNPVASFPDKNKVVRSLSKLKFMVVIDPLVTETSTEWQ
NHGESNDVDPASIQTEVFRLPSTCFAEEDGSIANSGRWLQWHWKGQDAPGEARNDGEILAGIYHRL
REMYRTEGGKGAEPLLKMRWSYKQPDHPESEEVAKENNGYALADLYDANGVLIAKKGQLLSSFAQL
RDDGTTSSSCWIYTGSWTEQGNQMANRDNADPSGLGNTLGWAWAWPLNRRVLYNRASADINGKPWD
PKRMLIQWNGAKWTGNDIPDFNTAPPGSKTGPFIMQPEGVGRLFALDKLAEGPFPEHYEPMETPLG
TNPLHPNVVSSPVVRIYEDDVLRLGKKDKFPYVGTTYRLTEHFHTWTKHALLNAIAQPEQFVEISE
TLAAAKGITNGDRVTVSSKRGFIRAVAVVTRRLRTLNVNGQQVETVGIPLHWGFEGVARKGYIANT
LTPNVGDSNSQTPEYKAFLVNIEKA
SEQ ID NO: 73-Escherichia alba Formate dehydrogenase-N subunit
alpha (fdnG)
MDVSRRKFFKICAGGMAGTTVAALGFSPKMALAQSRNYKLLRAKEIRNSCTYCSVGCGLLMYSLGD
GAMNAKEAIYHIEGDPDHPVSRGALCPKGAGLLDYVHSENRLRYPEYRAPGSNKWQRISWNEAFDR
IAKLMKADRDANFIEKNEQGVTVNRWLSTGMLCASAASNETGMLTQKFARSLGMLAVDNQARVUHG
PTVASLAPTFGRGAMTNHWVDIKNANVVMVMGGNAAEAHPVGFRWAMEAKNHNDATLIVVDPRFTR
TASVADIYAPIRSGTDITFLSGVLRYLIENNRINAEYVRHYTNAGLLVRDDFTFEDGLFSGYDAEK
RQYDKSSWNYQFDENGHALRDDTLTHPRCVWNLLKQHVSRYTPEVVENICGTPKADFLKVCEVLAS
TSAADRTTTFLYALGWTQHTVGAQNIRTMAMIQLLLGNMGMAGGGVNALRGHSNIQGLTDLGLLST
SLPGYLTLPSEKQTSLQSWLADNTPKATLPDQVNYWSNYPKFAVSLMKAFYGDDARKENDWGEDWL
PKWDQTYDVIKYFNMMDSGKVTGYICQGFNPVASFPDKNKVVRSLSKLKYMVVIDPLVTETSTEWQ
NHGESNDVDPASIQTEVFRLPSTCFAEEDGSIANSGRWLQWHWKGQDAPGEARNDGQILAGLFHRL
REMYRTEGGKGAEPLLKMRWDYKQPNHPESEEVAKENNGYALEDLYDANGKLIAKKGQLLSSFAQL
RDDGTTASSCWIYTGSWTEQGNQMANRDNADPSGLGNTLGWAWAWPLNRRVLYNRASADINGKPWD
PKRMLIQWDGKKWTGNDIPDFNTAAPGSKTGPFIMQPEGLGRLFALNKLAEGPFPEHYEPMETPLG
TNPLHPNVISSPVVRLYEEDAVRMGKKEQFPYVGTTYRLTEHFHTWTKHAKLNAIAQPEQFVEISE
TLAAAKNIANGDRVTVSSKRGFIRAVAVVTRRLRTLHVNGQQVETIGIPLHWGYEGVARKGYIANT
LTPNVGDSNSQTPEYKAFLVNIEKA
SEQ ID NO: 74-Enterobacteriaceae bacterium Formate
dehydrogenase-N subunit alpha (fdnG)
MDVSRRKFFKICAGGMAGTTAAALGFAPKMALAQARNFKLLRAKEIRNTCTYCSVGCGLLMYSLGD
GAKNAKEAIYHIEGDPDHPVSRGALCPKGAGLLDYVHSENRLRYPQYRAPGSDKWQRISWDEAFNR
IARLMKADRDANFIEKNEQGVTVNRWLSTGMLCASAASNETGMLTQKFVRSLGMLAVDNQARVUHG
PTVASLAPTFGRGAMTNHWVDIKNANVVVVMGGNAAEAHPVGFRWAMEAKNNNDATLIVVDPRETR
TASVADIYAPIRSGTDITFLSGVLLYLIENNKINAEYVKHYTNASLLVRDDFAFEEGLFSGYDAEK
RQYDKSSWNYQFDENGYAKRDKTLSHPRCVWNLLRQHVSRYTPEVVENICGTPKADFLKVCEVLAS
TSAADRTTTFLYALGWTQHTVGAQNIRTMAMIQLLLGNMGMAGGGVNALRGHSNIQGLTDLGLLST
SLPGYLTLPSDKQSDLQSYLSANTPKATLPDQVNYWSNYPKFFVSLMKSFYGEAAQKENDWGENWL
PKWDQAYDVIKYFNMMDNGNVTGYICQGENPVASFPDKNKVVRSLSKLKYLVVIDPLVTETSTFWQ
NHGESNDVDPAAIQTEVERLPSTCFAEEDGSIANSGRWLQWHWKGQDAPGEARNDGEILAGIYHRL
RELYRREGGKGAEPLLKMSWSYKQPDHPESAEVAKENNGYALADLYDQNGALLAKKGQLLNSFALL
RDDGSTASSCWIYTGSWTEQGNQMANRDNADPSGLGNTLGWAWAWPLNRRVLYNRASADINGKPWD
AKRMLIQWNGSKWVGNDIPDFNTAPPGSNTGPFIMQQEGLGRLFALDKLAEGPFPEHYEPMETPLG
TNPLHPKVVSSPVVRLYEEDAIRLGKKDKFPYVGTTYRLTEHFHTWTKHALLNSIAQPEQFVEISE
GLAKSKGIANGDWVKVSSKRGFIRAVAVVTRRLRTLNVNGQQVETVGIPLHWGFEGVARKGYIANT
LTPNVGDSNSQTPEYKAFLVNIEKA
SEQ ID NO: 75-Enterobacteriaceae bacterium 4M9 Formate
dehydrogenase-N subunit alpha (fdnG)
MDVSRRGFFKICAGGMAGTTVAVLGFAPKTALAQARNYKLLRAKEIRNTCTYCSVGCGLLMYSMGD
GSKNAKSSIFHIEGDPDHPVSRGALCPKGAGLLDYIHSDNRLRYPQYRAPGSDKWQRISWDDAFKR
IARLMKDDRDANFVETNDQGVKVNRWLSTGMLCASAASNETGMLTQKFARSLGMLAVDNQARVUHG
PTVASLAPTFGRGAMTNHWVDIKNANVVVVMGGNAAEAHPVGERWAMEAKNNNDATLIVVDPRETR
TASVADIYAPIRSGTDITFLSGVLLYLINNNKIHADYVKHYTNASLLIRDDYSFDDGLFSGYDADK
RQYDKTSWNYQFDENGHALRDMTLTHPRCVWNLLKEHVSRYTPDVVENICGTPKADFLKVCEVLAS
TSAANRAATFLYALGWTQHTVGAQNIRTMAMIQLLLGNMGVAGGGVNALRGHSNIQGLIDIGLLST
SLPGYLTLPQESHHDVETYLAANTPKALLPGQVNYWSNYPKFYVSLMKTFYGDAATRENGWGYDWL
PKWDQAYDVLKYFDMMDRGEVTGYFCQGFNPVASFPDKNKIVASLSKLKYMVVVDPLVTETSNEWQ
NHGEMNDVDPASIQTEVERLPSTCFAEEDGSIANSGRWLQWHWKGADAPGEAINDGQILAGLYDEL
RRLYRAEGGKGVEPLMKMSWNYKQPHEPESEEVAKENNGYALADLEDANGNLLVKKGELLDSFAQL
RDDGTTASGCWIYAGSWTRKGNQMANRDNADPSGLGNTLGWTWAWPLNRRVLYNRASCDPQGKAWD
PHRLLIEWNGEKWVGNDIPDYSAAAPELNAGPFIMQQEGLGRLFALNKLAEGPFPEHYEPIETPIG
TNPLHPNVVSSPVARMFKADAARIGDRKEFPYIGTTYRLTEHFHTWTKHARLNAIAQPEQFVEISE
ALAKSKGISNGDSVKVSSKRGFIRAKAVVTRRLQTFNVNGQQVETVGIPIHWGFEGKTQKGFVANT
LTPSVGDANSQTPEYKAFLVNIEKA
SEQ ID NO: 76-Erwinia sp. Formate dehydrogenase-N subunit alpha
(fdnG)
MDVSRRSFFKICAGGMAGTTVAALGFAPTTALAEARSYKLLRAKETRNTCTYCSVGCGLLMYSLGD
GAKNAKASIFHIEGDPDHPVNRGALCPKGAGLLDYIHSESRLRYPEYRAPGSDKWQRISWEDAFER
IAKLMKADRDANFISQNKQGTTVNRWLTTGMLCASAASNETGMLTQKFVRSLGMLAVDNQARVUHG
PTVASLAPTFGRGAMTNHWVDIKNANVVVVMGGNAAEAHPVGFRWAMEAKNNNDATLIVVDPRETR
TASVADIYAPIRSGTDITFLSGVLHYLITHNKINAEYVKHYTNAPLLVRDDFRFEDGLFSGYDAEK
RQYDKSSWNYQLDENGFARRDDTLSHPRCVWNLLKQHVSRYTPDVVENICGTPQADFLKVCEVLAS
TSAVDRITTFLYALGWTQHSVGSQNIRTMAMIQLLLGNMGMAGGGVNALRGHSNIQGLTDLGLLST
SLPGYLTLPKDDQHDLESYLAANTPKATLPGQVNYWGNYPKFFVSLMKTFWGDTATAQNQWGYDWL
PKWDQAYDVLNYFDMMDRGEVNGYICQGFNPVASFPDKNKVVASLSKLKYMVVIDPLVTETSNEWQ
NHGESNDVDPSQIQTEVFRLPSTCFAEEDGSIVNSGRWLQWHWKGADAPGEARNDGEILAGIYHRL
RTLYREQGGVASEPVLNMTWDYKLPDGPESEEVAKENNGYALADIHDASGKLLVKKGELLDSFAQL
RDDGTTAAGCWIWTGSWIRDGNQMARRDNADPSGLGNTLAWAWAWPLNRRVLYNRASADPAGKAWD
PKRVLIEWNGKKWVGNDVPDYNTAAPSENVGPFIMQQEGLGRLFALNKLAEGPFPEHYEPFETPLG
TNPLHPNVISNPVARLYQADAARLGERKAFPYVGTTYRLTEHFHTWTKHARLNAIAQPEQFVEISE
GLAKAKGIANGDTVKVSSKRGFIRAKAVVTRRLRTLEVNGQQVETVGIPLHWGFEGATRKGYLANT
LTPSVGDANSQTPEYKAFLVNIEKA
SEQ ID NO: 77-Jejubacter calystegiae Formate dehydrogenase-N
subunit alpha (fdnG)
MDVSRRSFFKICAGGMAGTTVAALGFAPTTALAEARSYKLLRAKETRNTCTYCSVGCGLLMYSLGD
GAKNAKSSIFHIEGDPDHPVNRGALCPKGAGLLDYIHSESRLRYPEYRAPGSDKWQRISWEDAFER
IAKLMKADRDANFISQNKQGTTVNRWLITGMLCASAASNETGMLTQKFVRSLGMLAVDNQARVUHG
PTVASLAPTFGRGAMTNHWVDIKNANVVVVMGGNAAEAHPVGFRWAMEAKNNNDATLIVVDPRETR
TASVADIYAPIRSGTDITFLSGVLHYLIAHNKINAEYVKHYTNAPLLVRDDFSFEDGLFSGYDENK
RQYDKSSWNYQLDESGFAKRDDTLSHPRCVWNLLKQHVSRYTPEVVENICGTPQADFQKVCEVLAS
TSAVDRTTTFLYALGWTQHSVGSQNIRTMAMIQLLLGNMGMAGGGVNALRGHSNIQGLTDLGLLST
SLSGYLTLPKEDQHDLESYLTANTPKATLPGQVNYWGNYPKFFVSLMKTFWGDAATAQNQWGYDWL
PKWDQAYDVLNYFDRMDRGEVNGYICQGFNPVASFPDKNKVVASLSKLKYMIVIDPLVTETSNEWQ
NHGESNDVDSSQIQTEVERLPSTCFAEEDGSIVNSGRWLQWHWKGADAPGEARNDGEILAGIYHRL
RTLYREQGGVASEPVLNMTWDYKLPDGPESEEVAKENNGYALADLYDADGKLLVKKGELLDSFAQL
RDDGTTASGCWIWTGSWTRNGNQMAKRDNADPSGLGNTLDWAWAWPLNRRVLYNRASADPAGKAWD
PKRVLIEWNGSKWVGNDVPDYNTAAPSQNVGPFIMQQEGLGRLFALNKLAEGPFPEHYEPFETPLG
TNPLHPAVISNPVARLYKADAARLGERKAFPYVGTTYRLTEHFHTWTKHARLNAIAQPEQFVEISE
GLAKAKGIANGDTVKVSSKRGFIRAKAVVTRRLRTLDVNGQQVETVGIPLHWGFEGATRKGYLANT
LTPSVGDANSQTPEYKAFLVNIEKA
SEQ ID NO: 78-Moellerella wisconsensis Selenocysteine-containing
formate dehydrogenase N alpha subunit (M992_0960)
MNLSRRQFFKICAGGMASTTIAALGFMPGLAMANVREYKLLRSKETRNTCTYCSVGCGLLIYSMGD
GAMNAKSSIFHIEGDADHPVNRGALCPKGAGLIDYIHSEGRLRYPEYRAPGSNKWQRISWDDAFSR
IARLMKDDRDTNFIEKNSQGISVNRWLSTGMLCASAASNETGILTQKFTRSLGMLAVDNQARVUHG
PTVASLAPTFGRGAMTNHWVDIKNANVIMVMGGNAAEAHPVGERWAMEAKNNNDATLIVVDPRFTR
TASVADHYVPIRSGTDITFLSGVLRYLIENNKINVEYVKHYTNASLLIRDDFSEDDGLESGYDAEK
RQYDKTSWNYQFDENGFAKRDDSWQHPRCVWNLLKQHISRYTPEIVENICGTSQADFLKVCEILAT
TSSADRITTFLYALGWTQHTVGSQNIRTMAMIQLLLGNMGMAGGGINALRGHSNIQGLTDLGLLST
SLPGYLSLPSEKQTNIESYLQANTPVATLPNQVNYWSNYPKFFVSLMKSFYGDAAQKDNQWGFDWL
PKWDQSYDVIKYFDMMSKGQVTGYICQGFNPVASFPDKNKVVSCLSKLKYLVIIDPLVTETSTFWQ
HHGEMNDVDPATIQTEVFRLPSTCFAEEDGSIANSGRWLQWHWKAADAPGEARNDGQILAGLLDKI
RQLYASEGGKGTEPLMRMRWDYKQPFHPESEEMAKENNGIALVDLYDADGNLQAKKGQLLNSFALL
RDDGSTASSCWIYSGCWTEQGNQMAKRDNSDPSGLGNTLGWAWAWPLNRRIIYNRASADPQGKPWD
KNRVVIEWKGNKWVGNDVPDFNTSAPDVGTSPFIMQPEGLGRLFAIDKMAEGPFPEHYEPVETPLG
TNPLHPNVVSNPAARIFLEDQQRLGNHQDYPYVGTTYRLTEHFHTWTKHARLNAIAQPEQFVEISQ
TLAQQKGINNGDKVTVSSKRGFIRAIAVVTARLQALHVDGKSIETIGIPIHWGFEGQAQKGFIANT
LTPSVGDANSQTPEYKAFLVNIEKA

1-25. (canceled)

26. A method of reducing a nitrogen-containing functional group in a target compound, comprising contacting the target compound with a biocatalyst comprising an oxidoreductase enzyme or a functional fragment or derivative thereof supported on a support material, in the presence of a molecular reductant for oxidation by the oxidoreductase enzyme or functional fragment or derivative thereof, under conditions such that:
the molecular reductant is oxidised by the oxidoreductase enzyme or functional fragment or derivative thereof; and
the nitrogen-containing functional group is reduced at the support material.

27. The method of claim 26, comprising contacting the target compound with the support material.

28. The method of claim 26, wherein reduction of the nitrogen-containing functional group comprises direct electron transfer from the support material to the target compound.

29. The method of claim 26, comprising reducing the nitrogen-containing functional group to form an amine group or a quaternary ammonium group.

30. The method of claim 26, wherein the oxidoreductase enzyme or functional fragment or derivative thereof is in electronic contact with the support material.

31. The method of claim 26, wherein the oxidoreductase enzyme or functional fragment or derivative transfers electrons to the support material via an intramolecular electronically-conducting pathway.

32. The method of claim 31, wherein the intramolecular electronically-conducting pathway comprises a series of [FeS] clusters.

33. The method of claim 26, wherein the molecular reductant is selected from hydrogen, carbon monoxide, formate, isotopes thereof, and mixtures thereof.

34. The method of claim 33, wherein the molecular reductant comprises or consists of hydrogen or an isotope thereof.

35. The method of claim 26, wherein the supported biocatalyst comprises a hydrogenase enzyme or a functional fragment or derivative thereof.

36. The method of claim 35, wherein the hydrogenase is selected from or comprises
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromatium vinosum membrane bound hydrogenase (SEQ ID NOs: 18 and/or 19) or an amino acid sequence having at least 60% homology therewith;
x) the amino acid sequence of Salmonella enterica serovar Typhimurium LT2 nickel-iron hydrogenase 5 (SEQ ID NO: 20 and/or 21) or an amino acid sequence having at least 60% homology therewith;
xi) the amino acid sequence of Desulfovibrio vulgaris Miyazaki F hydrogenase (SEQ ID NO: 23 and/or 24) or an amino acid sequence having at least 60% homology therewith;
xii) the amino acid sequence of Clostridium beijerinckii SM10 (CbA5H) [FeFe]-hydrogenase (KX147468) (SEQ ID NO: 25); Clostridium beijerinckii ATCC 51743 [FeFe]-Hydrogenase (Cbei_4110) (SEQ ID NO: 26); Clostridium beijerinckii [FeFe]-Hydrogenase (Cbei_1773) (SEQ ID NO: 27); or Clostridium beijerinckii [FeFe]-Hydrogenase (Cbei_3796) (SEQ ID NO: 28) or an amino acid sequence having at least 60% homology therewith;
xiii) the amino acid sequence of Clostridium pasteurianum [FeFe]-Hydrogenase (hydA) (SEQ ID NO: 29) or an amino acid sequence having at least 60% homology therewith;
xiv) the amino acid sequence of Chlamydomonas reinhardtii [FeFe]-hydrogenase (hyd1) (SEQ ID NO: 30) or an amino acid sequence having at least 60% homology therewith;
xv) the amino acid sequence of Chlorella variabilis [FeFe]-hydrogenase (SEQ ID NO: 31 and/or 32) or an amino acid sequence having at least 60% homology therewith;
xvi) the amino acid sequence of Ralstonia eutropha soluble hydrogenase moiety (SEQ ID NOs: 33 and/or 34) or an amino acid sequence having at least 60% homology therewith;
xvii) the amino acid sequence of Rhodococcus opacus soluble hydrogenase moiety (SEQ ID NOs: 35 and/or 36) or an amino acid sequence having at least 60% homology therewith;
xviii) the amino acid sequence of Desulfovibrio fructosovorans membrane bound hydrogenase (SEQ ID NOs: 37 and/or 38) or an amino acid sequence having at least 60% homology therewith;
xix) the amino acid sequence of Clostridium acetobutylicum iron-iron hydrogenase (SEQ ID NOs: 39) or an amino acid sequence having at least 60% homology therewith;
xx) the amino acid sequence of Desulfomicrobium baculatum nickel-iron selenium hydrogenase (SEQ ID NOs: 40 and/or 41) or an amino acid sequence having at least 60% homology therewith;
xxi) the amino acid sequence of Hydrogenophilus thermoluteolus soluble hydrogenase moiety (SEQ ID NOs: 42 and/or 43) or an amino acid sequence having at least 60% homology therewith;
xxii) the amino acid sequence of Desulfovibrio gigas Periplasmic [NiFe]hydrogenase (SEQ ID NOs: 44 and/or 45) or an amino acid sequence having at least 60% homology therewith; or
xxiii) the amino acid sequence of Pyrococcus furiosus soluble alpha subunit (SEQ ID NOs: 46) or an amino acid sequence having at least 60% homology therewith;
or a functional fragment, derivative or variant thereof.

37. The method of claim 26, wherein the supported biocatalyst comprises a carbon monoxide dehydrogenase enzyme or a functional fragment or derivative thereof.

38. The method of claim 37, wherein the carbon monoxide dehydrogenase is selected from or comprises
i) the amino acid sequence of Desulfovibrio vulgaris Hildenborough Carbon monoxide dehydrogenase (cooS) (SEQ ID NO: 47) or an amino acid sequence having at least 60% homology therewith;
ii) the amino acid sequence of Desulfovibrio vulgaris Miyazaki Carbon monoxide dehydrogenase (DvMF) (SEQ ID NO: 48) or an amino acid sequence having at least 60% homology therewith;
iii) the amino acid sequence of Desulfovibrio psychrotolerans Carbon monoxide dehydrogenase (cooS) (SEQ ID NO: 49) or an amino acid sequence having at least 60% homology therewith;
iv) the amino acid sequence of Desulfoluna spongiiphila Carbon monoxide dehydrogenase (SAMN05216233) (SEQ ID NO: 50) or an amino acid sequence having at least 60% homology therewith;
v) the amino acid sequence of Halodesulfovibrio spirochoetisodalis Carbon monoxide dehydrogenase (SP90) (SEQ ID NO: 51) or an amino acid sequence having at least 60% homology therewith;
vi) the amino acid sequence of Desulfovibrio desulfuricans Carbon monoxide dehydrogenase (Ddes) (SEQ ID NO: 52) or an amino acid sequence having at least 60% homology therewith;
vii) the amino acid sequence of Desulfurivibrio alkaliphilus Carbon monoxide dehydrogenase (DaAHT2) (SEQ ID NO: 53) or an amino acid sequence having at least 60% homology therewith;
viii) the amino acid sequence of Pseudodesulfovibrio aespoeensis Carbon monoxide dehydrogenase (Daes) (SEQ ID NO: 54) or an amino acid sequence having at least 60% homology therewith;
ix) the amino acid sequence of Desulfovibrio alaskensis Carbon monoxide dehydrogenase (Dde_3028) (SEQ ID NO: 55) or an amino acid sequence having at least 60% homology therewith;
x) the amino acid sequence of Desulfovibrio ferrophilus Carbon monoxide dehydrogenase (DFE_2686) (SEQ ID NO: 56) or an amino acid sequence having at least 60% homology therewith;
xi) the amino acid sequence of Carboxydothermus hydrogenoformans Carbon monoxide dehydrogenase 2 (cooS2) (SEQ ID NO: 57) or an amino acid sequence having at least 60% homology therewith;
xii) the amino acid sequence of Desulfofundulus salinum Carbon monoxide dehydrogenase (cooS) (SEQ ID NO: 58) or an amino acid sequence having at least 60% homology therewith;
xiii) the amino acid sequence of Caldanaerobacter subterraneus Carbon monoxide dehydrogenase tengcongensis (TTE1708) (SEQ ID NO: 59) or an amino acid sequence having at least 60% homology therewith;
xiv) the amino acid sequence of Thermanaeromonas toyohensis Carbon monoxide dehydrogenase (SAMN00808754_0706) (SEQ ID NO: 60) or an amino acid sequence having at least 60% homology therewith;
xv) the amino acid sequence of Desulfobulbus sp. Carbon monoxide dehydrogenase (Gene:JT06_17280) (SEQ ID NO: 61) or an amino acid sequence having at least 60% homology therewith;
xvi) the amino acid sequence of Desulfotomaculum copahuensis Carbon monoxide dehydrogenase (A6M21_00615) (SEQ ID NO: 62) or an amino acid sequence having at least 60% homology therewith;
xvii) the amino acid sequence of Pelotomaculum propionicicum Carbon monoxide dehydrogenase (cooS2) (SEQ ID NO: 63) or an amino acid sequence having at least 60% homology therewith;
xviii) the amino acid sequence of Methylomusa anaerophila Carbon monoxide dehydrogenase (cooS2) (SEQ ID NO: 64) or an amino acid sequence having at least 60% homology therewith;
xix) the amino acid sequence of Sporomusa silvacetica Carbon monoxide dehydrogenase (cooS2) (SEQ ID NO: 65) or an amino acid sequence having at least 60% homology therewith;
xx) the amino acid sequence of Heliobacillus mobilis Carbon monoxide dehydrogenase (cooS) (SEQ ID NO: 66) or an amino acid sequence having at least 60% homology therewith;
xxi) the amino acid sequence of Desulfocucumis palustris Carbon monoxide dehydrogenase (DCCM_2691) (SEQ ID NO: 67) or an amino acid sequence having at least 60% homology therewith;
or a functional fragment, derivative or variant thereof.

39. The method of claim 26, wherein the supported biocatalyst comprises a formate dehydrogenase enzyme or a functional fragment or derivative thereof.

40. The method of claim 39, wherein the formate dehydrogenase is selected from or comprises
i) the amino acid sequence of Escherichia coli Formate dehydrogenase, nitrate-inducible, major subunit (fdnG) (SEQ ID NO: 68) or an amino acid sequence having at least 60% homology therewith;
ii) the amino acid sequence of Shigella flexneri Formate dehydrogenase-N, nitrate-inducible, alpha subunit (fdnG) (SEQ ID NO: 69) or an amino acid sequence having at least 60% homology therewith;
iii) the amino acid sequence of Enterobacteriaceae bacterium Formate dehydrogenase-N subunit alpha (fdnG) (SEQ ID NO: 70) or an amino acid sequence having at least 60% homology therewith;
iv) the amino acid sequence of Salmonella typhimurium Molybdopterin oxidoreductase (fdnG) (SEQ ID NO: 71) or an amino acid sequence having at least 60% homology therewith;
v) the amino acid sequence of Citrobacter rodentium Formate dehydrogenase, nitrate-inducible, major subunit (fdnG) (SEQ ID NO: 72) or an amino acid sequence having at least 60% homology therewith;
vi) the amino acid sequence of Escherichia alba Formate dehydrogenase-N subunit alpha (fdnG) (SEQ ID NO: 73) or an amino acid sequence having at least 60% homology therewith;
vii) the amino acid sequence of Enterobacteriaceae bacterium Formate dehydrogenase-N subunit alpha (fdnG) (SEQ ID NO: 74) or an amino acid sequence having at least 60% homology therewith;
viii) the amino acid sequence of Enterobacteriaceae bacterium 4M9 Formate dehydrogenase-N subunit alpha (fdnG) (SEQ ID NO: 75) or an amino acid sequence having at least 60% homology therewith;
ix) the amino acid sequence of Erwinia sp. Formate dehydrogenase-N subunit alpha (fdnG) (SEQ ID NO: 76) or an amino acid sequence having at least 60% homology therewith;
x) the amino acid sequence of Jejubacter calystegiae Formate dehydrogenase-N subunit alpha (fdnG) (SEQ ID NO: 77) or an amino acid sequence having at least 60% homology therewith;
xi) the amino acid sequence of Moellerella wisconsensis Selenocysteine-containing formate dehydrogenase N alpha subunit (M992_0960) (SEQ ID NO: 78) or an amino acid sequence having at least 60% homology therewith;
or a functional fragment, derivative or variant thereof.

41. The method of claim 26, wherein the oxidoreductase enzyme or functional fragment or derivative thereof is immobilised on the support material.

42. The method of claim 26, wherein the support material is electronically conductive or semi-conductive.

43. The method of claim 26, wherein the support material comprises carbon, a metal or metal alloy, a metal oxide or mixed metal oxide, a metal hydroxide, a metal chalcogenide, a semi-conducting material, or an electronically-conductive polymer, or mixtures thereof.

44. The method of claim 26, wherein the support material comprises or consists of a carbon material.

45. The method of claim 44, wherein the carbon material comprises graphite, carbon nanotube(s), carbon black, activated carbon, carbon nanopowder, vitreous carbon, carbon fibre(s), carbon cloth, carbon felt, carbon paper, graphene, highly oriented pyrolytic graphite, pyrolytic graphite, doped or surface-modified carbon or doped diamond.

46. The method of claim 44, wherein the carbon material comprises:
doped graphene, wherein said graphene is doped with one or more dopants selected from nitrogen, boron, sulphur, oxygen, silicon, lanthanide elements and transition-metals;
doped carbon nanotube(s), wherein said carbon nanotube(s) are doped with one or more dopants selected from nitrogen, boron, sulphur, oxygen, silicon, lanthanide elements and transition-metals;
doped diamond, wherein said diamond is doped with one or more dopants selected from nitrogen, boron, sulphur, oxygen and silicon;
doped carbon black, wherein said carbon black is doped with one or more dopants selected from nitrogen, boron, sulphur, oxygen, silicon, lanthanide elements and transition-metals; and/or
doped activated carbon, wherein said activated carbon is doped with one or more dopants selected from nitrogen, boron, sulphur, oxygen, silicon, lanthanide elements and transition-metals;
and/or wherein said carbon material comprises or is modified to comprise carboxylic acid surface groups.

47. The method of claim 26, wherein the nitrogen-containing functional group is a nitro, an azide, a hydroxylamine, a nitroso, a nitrile, a diazo, a diazonium, an isocyanide, an isothiocyanate, an isocyanate, a hydrazone, a hydrazine, an amidine, an azo, or a guanidine group.

48. The method of claim 26, wherein the target compound is a nitroaromatic compound.

49. The method of claim 48, wherein the nitroaromatic compound comprises a hydrocarbyl aromatic group or a heteroaromatic group substituted with to a nitro group, wherein said hydrocarbyl aromatic group or a heteroaromatic group is optionally further substituted.

50. The method of claim 26, wherein the biocatalyst does not comprise a oxidoreductase enzyme or functional fragment or derivative thereof comprising an active site capable of catalysing the enzymatic reduction of the nitrogen-containing functional group.

51. The method of claim 50, wherein the biocatalyst does not comprise a nitro reductase enzyme.

52. The method of claim 26, wherein said method does not comprise transfer of electrons to the target compound via one or more cofactors.

53. A system, comprising:
i) a biocatalyst comprising an oxidoreductase enzyme or a functional fragment or derivative thereof supported on a support material;
ii) a molecular reductant; and
iii) a target compound comprising a reducible nitrogen-containing functional group;
wherein the system is configured such that, in use, (a) the molecular reductant is oxidised by the oxidoreductase enzyme or functional fragment or derivative thereof; and (b) the nitrogen-containing functional group is reduced at the support material.

54. The system of claim 53, wherein the oxidoreductase enzyme or functional fragment or derivative transfers electrons to the support material and the reduction of the nitrogen-containing functional group comprises direct electron transfer from the support material to the target compound.

55. The system of claim 53, wherein:
the oxidoreductase enzyme or a functional fragment or derivative thereof is in electronic contact with the support material, and/or transfers electrons to the support material via an intramolecular electronically-conducting pathway; and/or the supported biocatalyst comprises a hydrogenase enzyme or a functional fragment or derivative thereof, and/or a carbon monoxide dehydrogenase enzyme or a functional fragment or derivative thereof, and/or a formate dehydrogenase enzyme or a functional fragment or derivative thereo; and/or the oxidoreductase enzyme or functional fragment or derivative thereof is immobilised on the support material; and/or
the support material is electronically conductive or semi-conductive, and/or comprises carbon, a metal or metal alloy, a metal oxide or mixed metal oxide, a metal hydroxide, a metal chalcogenide, a semi-conducting material, or an electronically-conductive polymer, or mixtures thereof; and/or
the molecular reductant is selected from hydrogen, carbon monoxide, formate, isotopes thereof, and mixtures thereof; and/or
the target compound is a nitroaromatic compound and/or the nitrogen-containing functional group is a nitro, an azide, a hydroxylamine, a nitroso, a nitrile, a diazo, a diazonium, an isocyanide, an isothiocyanate, an isocyanate, a hydrazone, a hydrazine, an amidine, an azo, or a guanidine group; and/or
wherein the biocatalyst does not comprise a oxidoreductase enzyme or functional fragment or derivative thereof comprising an active site capable of catalysing the enzymatic reduction of the nitrogen-containing functional group.
